(19) United States
(12) Patent Application Publication
(10) Pub. No.: US 2007/0082876 A1
(43) Pub. Date: Apr. 12, 2007

(54) Novel C18 modified retrosteroids as progesterone receptor modulator compounds

(75) Inventors: Joseph Messinger, Sehnde (DE); Heinrich-Hubert Thole, Hannover (DE); Bettina Husen, Hannover (DE); Christiane Boecker, Hannover (DE); Maria Hinaje, Nancy (FR); Monika Buchholz, Langenfeld (DE); Christoph Mark, Worms (DE); Vibhuti Klingler-Dabral, Griesheim (DE)

Correspondence Address: CROWELL & MORING LLP;INTELLECTUAL PROPERTY GROUP, P.O. BOX 14300, WASHINGTON, DC, 20044-4300, US

(73) Assignee: Solvay Pharmaceuticals GmbH, Hannover (DE)

(21) Appl. No.: 11/529,628

(22) Filed: Sep. 29, 2006

Related U.S. Application Data
(60) Provisional application No. 60/722,689, filed on Sep. 30, 2005.

(30) Foreign Application Priority Data
Jul. 28, 2006 (EP) 06118034.5

(51) Int. Cl. A61K 31/58; A61K 31/57; C07J 43/00; C07J 5/00

(52) U.S. Cl. 514/176; 514/177; 540/107; 552/574

(57) ABSTRACT

Retrosteroidal compounds of formula I which act as progesterone receptor modulators, a method for their production, and pharmaceutical preparations containing these compounds. These compounds are preferably used for the treatment of benign gynecological disorders such as endometriosis and uterine fibroids, as well as for female birth control and for hormone replacement therapy (HRT).

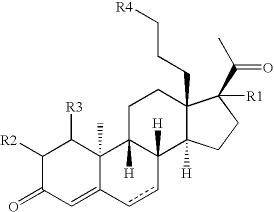

### CROSS REFERENCE TO RELATED APPLICATIONS

This application claims priority from U.S. Provisional Patent Application No. 60/722,689, filed Sep. 30, 2005, the entire disclosure of which is incorporated herein by reference. Paris Convention priority is also claimed based on European Patent Application No. EP 06118034.5, filed Jul. 28, 2006.

### FIELD OF THE INVENTION

The present invention relates to novel retrosteroidal derivatives that may be modulators (i.e., agonists, partial agonists and antagonists) of progesterone receptors, to their salts, to pharmaceutical preparations containing these compounds, to processes for the preparation of these compounds, and to uses of said compounds. The invention relates to the use of a compound disclosed herein for the manufacture of a medicament giving a beneficial effect, whereby a beneficial effect is disclosed herein or apparent to a person skilled in the art from the specification and general knowledge in the art. The invention also relates to the use of a compound of the invention for the manufacture of a medicament for treating or preventing a disease or condition. More particularly, the invention relates to a new use for the treatment of a disease or condition disclosed herein or apparent to a person skilled in the art from the specification and general knowledge in the art. In embodiments of the invention specific compounds disclosed herein are used for the manufacture of a medicament useful in the treatment of disorders or conditions mediated by progesterone receptors, or of disorders or conditions that can be treated via modulation of those receptors. In particular, the invention concerns the therapeutic use of said novel retrosteroidal derivatives in the treatment or prevention of benign gynecological disorders, especially endometriosis, uterine fibroids, and dysfunctional uterine bleeding, in hormonal female contraception or in hormone replacement therapy.

### BACKGROUND OF THE INVENTION

The publications and other materials used herein to illuminate the background of the invention, and in particular, cases to provide additional details respecting the practice, are incorporated by reference to the same extent as if each reference there individually and specifically indicated to be incorporated by reference and were set forth in its entirety herein herein and are not admitted to be prior art.

Progesterone and the Progesterone Receptor

Progesterone is secreted in large amounts from the ovary or the placenta during the cycle and in pregnancy. In combination with estrogens, progesterone produces cyclic changes of the mucous membrane of the uterus in the menstrual cycle. In pregnancy, progesterone controls the relaxation of the myometrium and preserves the function of the decidual tissue. Under the influence of elevated progesterone levels after ovulation, the mucous membrane of the uterus is converted into a state that allows the nidation of an embryo (blastocyst). In a subtle way, progesterone is involved in the control of ovulation processes. It is known that progesterone has anti-ovulatory properties in connection with estrogens. The latter finding results from an inhibition of the hypophyseal gonadotropin secretion, which is a requirement for the maturation of a follicle and its ovulation. In contrast, it is evident that the comparatively low progesterone secretion of the maturing follicle plays an active role for the preparation and triggering of ovulation. In this connection, hypophyseal mechanisms (time-limited so-called positive feedback of progesterone on gonadotropin secretion) play a significant role. In addition, it is known that progesterone exerts a decisive influence on the endometrium. The endometrial proliferation is inhibited by the suppression of the estrogen-mediated mitosis in the uterus tissue.

PR Modulators and SPRMs

Within the scope of the present invention, progesterone receptor (PR) modulators comprise compounds which may be agonists showing high affinity and/or high specificity, partial agonists (i.e., partial activators and/or tissue-specific activators) and/or antagonists for PRs, whereby the term PR always comprises the progesterone receptor alpha (PRQ) and/or the progesterone receptor beta (PRY) isoforms. Generally spoken is a compound that binds to the PR and mimics the action of the natural hormone, i.e. progesterone, termed an agonist, whilst a compound which inhibits the effect of said natural ligand is an antagonist. Preferably, the (selective) PR modulators—usually called SPRMs—possess both agonistic and antagonistic activities at the PR measured in-vitro, e.g. using assays of progesterone dependent enzymes in PR expressing cell lines, and/or determined in vivo, e.g. using the classical bioassay, the McPhail test, which assesses progestagenic and antiprogestagenic effects in rabbits [McPhail, 1934]. A typical in-vitro assay to determine agonistic and antagonistic activities of the compounds at the PR is the so-called “AP assay” (a progesterone-dependent endogenous alkaline phosphatase (AP) expression assay) using the human mammary carcinoma T47D cell line [Di Lorenzo, 1991 and Sobek, 1994].

An even more sophisticated definition of SPRMs—as mesoprogestins—is given within WO 01/15679: As combined progestins (PR agonist) and anti-progestins (PR antagonists), mesoprogestins show high binding affinity to PR, but exhibit different pharmacodynamic properties compared to either pure progestins or antiprogestins. Mesoprogestins possess progesterone agonistic activity which can be measured in vitro or in commonly used biological tests in vivo; however, this activity remains below that of natural progesterone in the plateau of the dose response curve. Accordingly, mesoprogestins stabilize the function of the PR at an intermediate activity level providing the rationale for the different clinical applications in gynecological therapy.

In the classical bioassay, the McPhail test, which assesses progestagenic and antiprogestagenic effects in rabbits [McPhail, 1934], progesterone produces a maximum McPhail score of 4 (by definition). According to the definition given within WO 01/15679, the treatment with a mesoprogestin in the absence of progesterone leads, however, to a McPhail score which is higher than that under any dose of RU 486 (Mifepristone), i.e. above 0.5-1.0, preferentially above 2.0-3.0, but to a distinctly lower score than 4 at the plateau of the dose response curve at the clinically relevant doses (i. e. 0.01 mg-30 mg/rabbit). The capacity of mesoprogestins to antagonize progesterone function can also be tested in the McPhail test using a progesterone dose which induces a McPhail score ranging between 3 and 4. A SPRM inhibits the effect of progesterone to a significant degree, but the maximum inhibition is below that which is inducible with RU 486 or other pure antiprogestins, such as onapristone.

Preferred Indications

PR modulators have been widely used in regulation of female reproduction systems and in treatment of female hormone dependent diseases (e.g. reviewed in Spitz [2003, Steroids]. In particular, benign gynecological pathologies such as endometriosis, uterine leiomyomas (uterine fibroids or myomas), adenomyosis, dysfunctional uterine bleeding (menorrhagia and metrorrhagia) and dysmenorrhoea can be treated by the administration of PR modulators. Furthermore, SPRMs may also be useful for the treatment of endometrial hyperplasia, meningiomas, hormone-dependent cancers such as ovarian cancer, breast cancer, endometrial cancer and prostate cancer and female osteoporosis. SPRMs can also be used for female hormone replacement therapy, i.e. for the treatment of hormonal disorders in postmenopausal women such as e.g. hot flushes and/or mood disorders. In addition, SPRMs can be used in female contraceptives.

Endometriosis is a well-known gynaecological disorder that affects 10 to 15% of women in the reproductive age. It is a benign disease defined as the presence of viable endometrial gland and stroma cells outside the uterine cavity. It is most frequently found in the pelvic area. In women developing endometriosis, the endometrial cells entering the peritoneal cavity by retrograde menstruation (the most likely mechanism) have the capacity to adhere to and invade the peritoneal lining, and are then able to implant and grow. The implants respond to steroid hormones of the menstrual cycle in a similar way as the endometrium in the uterus. The infiltrating lesions and the blood from these lesions which are unable to leave the body cause inflammation of the surrounding tissue. The most common symptoms of endometriosis are primary or acquired dysmenorrhoea, dyspareunia and (chronic) pelvic pain, especially before and in the menstruation period. Further symptoms could include dysuria, various genitourinary symptoms secondary to urethral obstruction and/or bladder invasion, painful defecation, rectal pressure, defecation urgency and bowel obstruction, bleeding abnormalities, including menorrhagia or metrorrhagia, infertility, primary or secondary, recurrent spontaneous abortions. The occurrence of these symptoms is not related to the extent of the lesions. Some women with severe endometriosis are asymptomatic, while women with mild endometriosis may have severe pain. Up to now, no reliable non-invasive test is available to diagnose endometriosis. Laparoscopy has to be performed to diagnose the disease. Endometriosis is classified according to the 4 stages set up by the American Fertility Society (AFS). Stage I corresponds to minimal disease while stage IV is severe, depending on the location and the extent of the endometriosis. Endometriosis is found in up to 50% of the women with infertility. However, currently no causal relation has been proven between mild endometriosis and infertility. Moderate to severe endometriosis can cause tubal damage and adhesions leading to infertility. The aims of treatment of endometriosis are pain relief, resolution of the endometriotic tissue and restoration of fertility (if desired). The two common treatments are surgery or anti-inflammatory and/or hormonal therapy or a combination thereof.

Uterine leiomyomas (fibroids or myomas), benign clonal tumours, arise from smooth muscle cells of the human uterus. They are clinically apparent in up to 25% of women and are the single most common indication for hysterectomy. They cause significant morbidity, including prolonged and heavy menstrual bleeding, pelvic pressure and pain, urinary problems, and, in rare cases, reproductive dysfunction. The pathophysiology of myomas is not well understood. Myomas are found submucosally (beneath the endometrium), intramurally (within the myometrium) and subserosally (projecting out of the serosal compartment of the uterus), but mostly are mixed forms of these 3 different types. The presence of sex steroid receptors in leiomyoma cells has been studied by Tamaya et al. [1985]. They have shown that the ratios of estrogen receptor compared to progesterone and androgen receptor levels were higher in leiomyomas than in the corresponding normal myometrium. Surgery has long been the main treatment for myomas. Furthermore, medical therapies that have been proposed to treat myomas include administration of a variety of steroids such as the androgenic steroids danazol or gestrinone, GnRH agonists and progestogens, whereby the administration is often associated with a variety of serious side-effects.

Dysfunctional uterine bleeding disorders (dysfunctional or abnormal uterine bleeding, metrorrhagia and menorrhagia, hypermenorrhea) are forms of pathological bleeding that are not attributable to organic changes in the uterus (such as, e.g., endometrial carcinoma, myomas, polyps, etc.), systemic coagulation disorders, or a pathological pregnancy (e.g., ectopic pregnancy, impending abortion) [American College of Obstetricians and Gynecologists, 1982]. The average blood loss during normal menstruation is about 30 ml, whereby the period lasts for an average of 5 days. If the blood loss exceeds 80 ml, it is classified as pathological [Zahradnik, 1992]. Metrorrhagias are defined as bleeding that may or may not be accompanied by pain and that cannot be linked to menstruation or cycle. If it lasts over 7 days, the blood loss often exceeds 80 ml. Menorrhagia is menstruation that may or may not be accompanied by pain, normally every 27-28 days, which, when it lasts over 7 days, is associated in most cases with an increased blood loss of over 80 ml. Menorrhagia is a syndrome of unknown origin and one of the most common problems in gynecology. 60% of women refereed with menorrhagia have a hysterectomy within five years. Hypermenorrhea is defined as menstruation that may or may not be accompanied by pain, normally every 27-28 days for 4-5 days with an elevated blood loss of over 80 ml, sometimes even defined as associated with an increased blood loss of over 150 ml. Forms of dysfunctional uterine bleeding (mainly metrorrhagias and menorrhagias) are typical of adolescence and of the time of menopause, in which follicle-stimulating disorders, anovulation, and yellow-body and follicle persistence occur in clusters. The incidence of dysfunctional uterine bleeding is high and represents one of the most frequent reasons for gynecological consultation for women of reproductive age. The consultation rate because of dysfunctional uterine bleeding is 33% in reproductive age and 69% in perimenopause and postmenopause [Mencaglia et al. 1987].

Everything that has been said above in relation to the treatment of uterine leiomyomas, endometriosis and dysfunctional uterine bleeding, equally applies to other benign gynaecological disorders, notably adenomyosis and dysmenorrhea. These benign gynaecological diseases can be treated in a comparable way as described herein before in relation to uterine leiomyomas, endometriosis and dysfunctional uterine bleeding. The available pharmaceutical treatments, however, suffer from the same major drawbacks, i.e. they have to be discontinued once the side-effects become more serious than the symptoms to be treated and symptoms reappear after discontinuation of the therapy.

Known Compounds Acting as SPRMs

Several PR modulators of steroidal origin are known in the literature and have been recently reviewed, see e.g. Spitz [2005], Spitz [2003, Steroids], Spitz [2003, Expert Opin Invest Drugs]. Non-steroidal PR modulators have been reviewed by Zhang et al. [2003].

The so far best characterized PR modulator of steroidal origin is Asoprisnil (J-867)

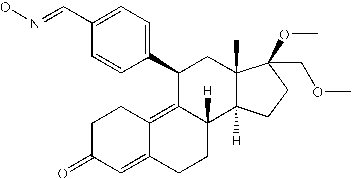

This compound belongs to the class of 11beta-benzaldoxime-substituted estratrienes that exhibit partial progesterone agonistlantagonist effects with high PR specificity in animals and humans [Schubert et al., 2005]. Asoprisnil (J867) has been described to be under development for the potential oral treatment of uterine fibroids and endometriosis.

The 11β-benzaldoxime-substituted estratrienes having the general structure shown below, in which R can be a hydrogen atom or an alkyl group and R1 can be a hydrogen atom, an alkyl group or aryl group or an optionally substituted acyl function, are known as PR modulators from EP 1229906 and EP 0648778:

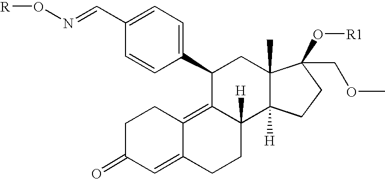

WO 99/45023 relates to S-substituted 11β-benzaldoxim-estra-4,9-diene-carboxylic acid-thiol ester. The compounds have antigestagenic properties while at the same time having an antigluocorticoidal action that is significantly more reduced in comparison to that of RU 486.

In EP 909764, 11β-benzaldoxime-9α,10α-epoxy-estr-4-ene derivatives with high binding affinity to the PR in the presence of low glucocorticoid receptor affinity are described.

WO 01/44267 describes new 11β-phenylestradiene derivatives with fluoroalkyl groups in the aromatic side chain and production thereof. The compounds or the pharmaceutical preparations that contain these compounds are antihormonally effective and are therefore suitable for the treatment of diseases that are unfavorably influenced by cortisol or by corticoids, for the reduction of secreted cortisol, for stimulation of lactation, for treating dysmenorrhea and myomas, for treating Cushing's disease and for cervical maturation, for improving cognitive performance, for treating endometriosis or for hormone replacement therapy (HRT).

WO 03/093292 discloses 17α-fluoroalkyl-11β-benzaldoxime-steroids and production thereof, pharmaceutical preparations that contain these steroids, especially for postmenopausal substitution therapy of gynecological diseases, such as hysteromyomas or dysmenorrhoic symptoms.

WO 04/014935 describes further substituted 11β-benzaldoxime-steroids, in particular 4-(3-oxo-estra-4,9-dien-11beta-yl)-benzaldehyde oximes, which are PR modulators useful in female contraception, hormone replacement therapy and treatment of gynecological disorders.

Further 11β substituted 17β-acyl-17α-propynyl steroids are known from WO 01/18025, whereas WO 00/34306 describes 17β-acyl-17α-propynyl-11β-arylsteroids and their derivatives having agonist or antagonist hormonal properties. In addition, WO 99/62928 discloses 17β-amino- and 17β-hydroxylamino-11β-arylsteroids, WO 99/62929 discloses 17β-nitro-11β-aryl-steroids and WO 99/45022 discloses 20-keto-11β-arylsteroids having agonist or antagonist hormonal properties.

The effectiveness of known steroidal SPRMs is often tempered by their undesired side-effect profile, particularly during long-term administration. For example, the effectiveness of synthetic progestins, such as Norgestrel, as female birth control agents must be weighed against the increased risk of breast cancer and heart disease. Similarly, the progesterone antagonist, mifepristone (RU 486), if administered for chronic indications, such as uterine fibroids, endometriosis and certain hormone-dependent cancers, could lead to homeostatic imbalances in a patient due to its inherent cross-reactivity as a glucocorticoid receptor (GR) antagonist.

Accordingly, identification of compounds which have good receptor-selectivity for the PR over other steroid hormone receptors, which provide a good tissue-selectivity (e.g. selectivity for uterine tissue over breast tissue) and which are agonists, partial agonists (i.e., partial activators and/or tissue-specific activators) and/or antagonists for PRs, which preferably show a balanced agonistic/antagonistic profile, would be of significant value in the improvement of women's health.

Known Retrosteroids

Retrosteroids, i.e. steroids with 9β, 10α conformation, are well known in the state of the art. The commercially available compound Dydrogesterone ((9β,10α)-Pregna-4,6-diene-3,20-dione) of the following formula 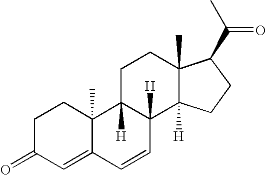
is an orally active progestative hormone and is generally used to correct deficiencies of progesterone in the body. The synthesis of Dydrogesterone by irradiation and photochemical reaction is for example described within European patents EP0152138B1 (U.S. Pat. No. 4,601,855) and EP0558119B1 (U.S. Pat. No. 5,304,291).

Further known retrosteroids with progestational activity are for example 1,2-methylene-3-keto-Δ^{4,6}-bisdehydro-6-halo-9β,10α-steroids as disclosed within U.S. Pat. No. 3,937,700 and 3-keto-Δ^{4,6}-bisdehydro-6-halo-9β,10α-steroids as described within BE 652,597 and U.S. Pat. No. 3,304,314. Furthermore, the patent application U.S. Pat. No. 3,555,053 describes a process for the preparation of 6-halo- or 6-alkyl-9β,10α-steroids. Some 6,7-dehydro-9β,10α steroids are described by Westerhof & Hartog [1965]. The synthesis of further retrosteroids is disclosed within Hartog et al. [1972] for some 16-methylene-17α-acetoxy-9β,10α-pregna-4,6-diene-3,20-dione derivatives and within Halkes et al [1972] for 1,2β-methylene-17α-acetoxy-9β,10α-pregnanes. In addition, 18-alkyl-9β,10α-pregnane derivatives are disclosed by Van Moorselaar & Halkes [1969]. However, the retrosteroidal compounds known so far were all developed for having progestational activity, i.e. being PR agonists.

Accordingly, there remains still a need for the development of novel compounds which therapeutically modulate the PR with an improved agonistic and/or antagonistic mode and with higher selectivity than currently known compounds. In particular, there is a need for selective PR modulators useful for the treatment of benign gynecological disorders such as endometriosis, uterine fibroids, uterine leiomyoma, endometrial hyperplasia, dysmenorrhea, and dysfunctional uterine bleeding (menorrhagia, metrorrhagia).

### SUMMARY OF THE INVENTION

The object of the present invention was to develop novel PR modulators based on the retrosteroidal core of the known progesterone agonist Dydrogesterone.

Another object of the invention was to develop compounds that combine the known beneficial properties of Dydrogesterone with novel modifications of the retrosteroidal core in order to obtain PR modulators, i.e. compounds with agonistic as well as antagonistic properties towards the PR, suited for the treatment of a broad range of gynaecological diseases requiring the modulation of the PR.

Surprisingly it has been found that the compounds of the invention represent PR modulators possessing agonistic and/or antagonistic activities at the PR in vivo. Accordingly, the present invention relates to compounds of general formula (I): 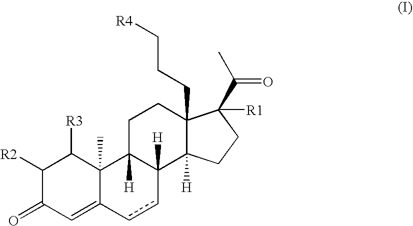
wherein
R1 is selected from hydrogen, —OH, —O—(C₁-C₄)alkyl, —O—CO—(C₁-C₄)alkyl, and —O—CO—O—(C₁-C₄)alkyl;
R2 and R3 are both hydrogen or together form a methylene group;
R4 is selected from —O—R⁶, heteroaryl and aryl;
   whereby the heteroaryl or the aryl group is optionally substituted with one or two substituents independently selected from the group consisting of
      —CHO; —CO—O—R⁹, —CO—NR¹²R¹³, —CH₂—O—R⁹; —CH₂—O—CO—R¹¹, —CH₂—O—CO—NHR¹², —CH═N—O—R¹⁴, —CH═N—O—CO—NHR¹², —CH═N—O—CO—R¹¹, —CH═N—O—CO—O—R¹⁴, —CN; —CH₂—NH—CO—NHR¹², —CH₂—NH—CO—R¹², —CH₂—NH—CO—O—R¹⁴, -halogen, —O—R⁹, —O—CO—R¹¹, —O—CO—NHR¹², —NR¹²R¹³; —NR¹⁰—CO—R¹¹, —NR¹⁰—O—CO—NHR¹², —NR¹⁰—O—CO—O—R¹⁴, —(C₁-C₄)alkyl, and halogenated —(C₁-C₄)alkyl,
   or which aryl is optionally substituted by two groups which are attached to adjacent carbon atoms and are combined into a saturated or partly unsaturated cyclic 5-, 6-, 7-, or 8-membered ring system, optionally containing 1, 2 or 3 heteroatoms selected from N, O and S, the number of N atoms being 0, 1, 2 or 3 and the number of O and S atoms each being 0, 1, or 2;
R⁶, R⁹, R¹⁰, R¹¹, R¹², R¹³ and R¹⁴ are independently selected from the group consisting of hydrogen, —(C₁-C₄)alkyl, and halogenated —(C₁-C₄)alkyl; or
R¹² and R¹³ form together with the nitrogen atom, where R¹² and R¹³ are attached, a heterocyclic 4-, 5-, 6-, 7- or 8-membered ring system, which is saturated, partly unsaturated, or aromatic; and which optionally contains 1, 2 or 3 additional heteroatoms selected from N, O and S, the number of additional N atoms being 0, 1, 2 or 3 and the number of O and S atoms each being 0, 1 or 2; and which ring is optionally part of a multiple condensed ring-system.

Pharmaceutically acceptable salts as well as all tautomers, stereoisomers, racemates, enantiomers of the compounds of the invention and mixtures thereof, unless the formula depicting the compound explicitly shows a particular stereochemistry, are also within the scope of the invention. Such isomers can be isolated by standard resolution techniques, including fractional crystallization and chiral column chromatography. Furthermore the compounds of the invention also include isotopically-labeled and radio-labeled compounds, as well as commonly used pro-drugs and active metabolites of these compounds.

Compounds of the invention include those represented by general formulae (II)

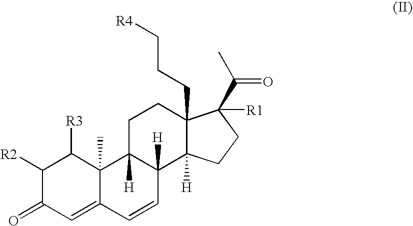

wherein R1 through R14 all have the same definitions as given above.

Furthermore, the present invention comprises compounds including those represented by general formulae (III) 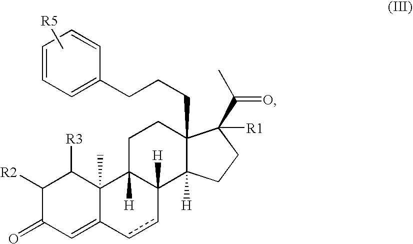
and those represented by general formula (V) 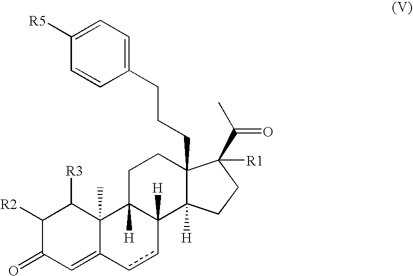
wherein for compounds of general formula (V)
R1 is selected from hydrogen, —OH, —O—(C₁-C₄)alkyl, —O—CO—(C₁-C₄)alkyl, and —O—CO—O—(C₁-C₄)alkyl;
R2 and R3 both are hydrogen or together form a methylene group;
R5 is selected from the group consisting of
   —CHO; —CO—O—R⁹, —CO—NR¹²R¹³, —CH₂—O—R⁹; —CH₂—O—CO—R¹¹, —CH₂—O—CO—NHR¹², —CH═N—O—R¹⁴, —CH═N—O—CO—NHR¹², —CH═N—O—CO—R¹¹, —CH═N—O—CO—O—R¹⁴, —CN; —CH₂—NH—CO—NHR¹², —CH₂—NH—CO—R¹¹, —CH₂—NH—CO—O—R¹⁴, -halogen, —O—R⁹, —O—CO—R¹¹, —O—CO—NHR¹², —NR¹²R¹³; —NR¹⁰—CO—R¹¹, —NR¹⁰—CO—NHR¹², —NR¹⁰—CO—O—R¹⁴, —(C₁-C₄)alkyl, and halogenated —(C₁-C₄)alkyl,
R⁹, R¹⁰, R¹¹, R¹², R¹³ and R¹⁴ are independently selected from the group consisting of hydrogen, —(C₁-C₄)alkyl and halogenated —(C₁-C₄)alkyl; or
R¹² and R¹³ form together with the nitrogen atom, where R¹² and R¹³ are attached, a heterocyclic 4-, 5-, 6-, 7- or 8-membered ring system, which is saturated, partly unsaturated, or aromatic; and which optionally contains 1, 2 or 3 additional heteroatoms selected from N, O and S, the number of additional N atoms being 0, 1, 2 or 3 and the number of O and S atoms each being 0, 1 or 2; and which ring is optionally part of a multiple condensed ring-system.

Compounds of the invention include those represented by general formulae (IV) 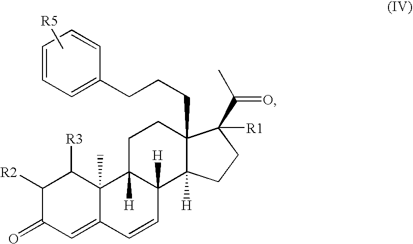
and those represented by general formula (VI)

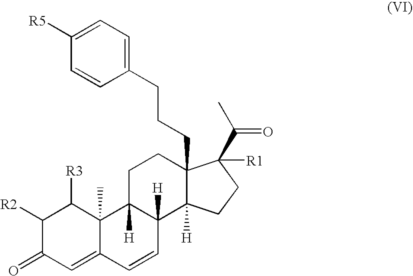

wherein for compounds of formula (IV) as well as for compounds of general formula (VI), R1 through R14 all have the same definitions as given above for compounds of general formula (III) and general formula (V).

In a further aspect, the present invention relates to a pharmaceutical composition comprising a pharmacologically active amount of at least one compound of the invention according to any one of formulae I through VI shown above wherein R1 through R14, and n all have the same definitions as given above, or a salt or pro-drug thereof, as an active ingredient and at least one pharmaceutically acceptable carrier and/or at least one pharmaceutically acceptable auxiliary substance.

Additionally, the invention relates to a compound of the invention or a salt or pro-drug thereof, for use as a medicament.

Furthermore, the invention relates to the use of a compound of the invention for the manufacture of a medicament for the treatment or prevention of a disorder or condition mediated by a PR, or that can be treated via modulation of that receptor.

In addition, the invention relates to the use of an effective amount of a compound of the invention for the treatment or prevention of a disorder or condition mediated by a PR, or that can be treated via manipulation of that receptor, in an individual, preferably in a mammal, in particular a human.

Preferably the disorder or condition mediated by a PR, or that can be treated via manipulation of that receptor is selected from: endometriosis, uterine fibroids, uterine leiomyoma, endometrial hyperplasia, dysmenorrhea, dysfunctional uterine bleeding, menorrhagia, metrorrhagia, hypermenorrhea, hot flushes, mood disorders, meningiomas, hormone-dependent cancer, in particular female sex steroid dependent cancer, ovarian cancer, breast cancer, endometrial cancer and prostate cancer; female osteoporosis, Cushing's syndrome, major depression, neurodegenerative diseases, Alzheimer's disease, and demyelinating diseases.

In a further aspect, the present invention relates to the use of a compound of the invention for the manufacture of a medicament for female birth control, for modulation of fertility or for female hormone replacement therapy (the treatment of hormonal disorders in postmenopausal women).

Furthermore, it will be understood by those skilled in the art that the compounds of the present invention, including pharmaceutical compositions and formulations containing these compounds, can be used in a wide variety of combination therapies to treat the conditions and diseases described above. Thus, the compounds of the present invention can be used in combination with other hormones, in particular estrogenic compounds and estrogen receptor modulators, and other therapies, including, without limitation, chemotherapeutic agents such as cytostatic and cytotoxic agents, immunological modifiers such as interferons, interleukins, growth hormones and other cytokines, hormone therapies, surgery and radiation therapy.

In particular, the pharmaceutical composition of the invention further comprises at least one low-dose natural or synthetic estrogen or pro-drugs thereof; preferably the estrogen is used as a natural estrogen, e.g. as a conjugated estrogen obtained from pregnant mare's urine (conjugated equine estrogen). Alternatively, the estrogen may be presented as the respective 3-sulfamate.

In one embodiment, the pharmaceutical composition of the present invention is in the form of an intrauterine device (IUD) ), in the form of a transdermal patch or a gel.

Furthermore, the invention also relates to a method of treating an individual, i.e. a mammal such as a human, having a condition mediated by a PR or which condition can be treated via modulation of that receptor, comprising administering to said individual an amount of a compound of this invention, or a salt or a pro-drug thereof, which amount is effective to treat the condition. Administration of compounds of this invention in combination with other pharmaceuticals used in treatment of the listed conditions is contemplated.

The conditions to be treated include but are not limited to endometriosis, uterine fibroids, uterine leiomyoma, endometrial hyperplasia, dysmenorrhea, dysfunctional uterine bleeding, menorrhagia, metrorrhagia, hypermenorrhea, hot flushes, mood disorders, meningiomas, hormone-dependent cancers, in particular female sex steroid dependent cancer, ovarian cancer, breast cancer, endometrial cancer and prostate cancer; female osteoporosis, Cushing's syndrome, major depression, neurodegenerative diseases, Alzheimer's disease, and demyelinating diseases. Additionally, the conditions to be treated may be alleviated with female hormone replacement therapy.

In a further aspect, the present invention relates to a method of modulating fertility (e. g., use of the compounds of the invention as contraceptive agents, contragestational agents or abortifacients, for in vitro fertilization, and for pregnancy maintenance) in an individual comprising administering to said individual a pharmaceutically effective amount of a compound of this invention, or a salt or a pro-drug thereof. Preferably, the present invention provides a method of contraception to an individual comprising administering to said individual a pharmaceutically effective amount of a compound of this invention, or a salt or a pro-drug thereof.

The compounds of the present invention, including pharmaceutical compositions and formulations containing these compounds, may be used in combination or conjunction with one or more estrogenic compounds or estrogen receptor modulators, in particular for female hormone replacement therapy, as modulators of fertility and in treatment of female osteoporosis.

According to a further aspect of the invention, a method is disclosed of modulating a PR in an individual comprising administering to said individual a compound of this invention, or a salt or a pro-drug thereof, in an amount effective to modulate a PR. Preferably, said modulation is activation.

Additionally, the compounds of this invention also have utility when e.g. radio- or isotopically labelled as ligands for use in assays to determine the presence of PR in a cell background or extract. They are particularly useful due to their ability to selectively activate PRs, and can therefore be used to determine the presence of such receptors in the presence of other steroid receptors or related intracellular receptors. Therefore, the present invention also relates to a method of determining the presence of a progesterone receptor (PR) in a cell or cell extract comprising (a) labeling a compound of this invention, or a salt or a pro-drug thereof; (b) contacting the cell or cell extract with said labeled compound; and (c) testing the contracted cell or cell extract to determine the presence of progesterone receptor.

### DETAILED DESCRIPTION

Definitions:

The following terms are used to describe the present invention. The terms are defined with the following meanings, unless explicitly stated otherwise:

The use of the terms “a” and “an” and “the” and similar referents in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope of the claims. No language in the specification should be construed as indicating any non-claimed element as essential to the practice of the invention.

Alternative embodiments of the claimed invention are described herein, including the best mode known to the inventors for carrying out the claimed invention. Of these, variations of the disclosed embodiments will become apparent to those of ordinary skill in the art upon reading the foregoing disclosure. The inventors expect skilled artisans to employ such variations as appropriate, and the inventors intend for the invention to be practiced otherwise than as specifically described herein.

Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

Individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the claimed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Thus, recitation of ranges of values herein are merely intended to serve as a shorthand method of referring individually to each separate value falling within the range, unless otherwise indicated herein, and each separate value is incorporated into the specification as if it there individually recited herein.

It is to be understood that any ranges, ratios and ranges of ratios that can be formed by, or derived from, any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art most closely related to a particular range, ratio or range of ratios will appreciate that such values are unambiguously derivable from the data presented herein.

The terms “comprising” and “including” are used herein in their open, non-limiting sense.

The word “compound” shall here be understood to cover any and all isomers (e. g., enantiomers, stereoisomers, diastereomers, rotomers, tautomers) or any mixture of isomers, pro-drugs, and any pharmaceutically acceptable salt of said compound, unless the formula depicting the compound explicitly shows a particular stereochemistry.

Where the plural form is used for compounds, salts, and the like, this is taken to mean also a single compound, salt, or the like.

The term “pro-drug” as used herein, represents derivatives of the compounds of the invention that are drug precursors which, following administration to a patient by any known route, release the more active metabolite drug in vivo via a chemical or physiological process. Pro-drugs are bioreversible derivatives of drug molecules used to overcome some barriers to the utility of the parent drug molecule. These barriers include, but are not limited to, solubility, permeability, stability, presystemic metabolism and targeting limitations (Medicinal Chemistry: Principles and Practice, 1994, ISBN 0-85186-494-5, Ed.: F. D. King, p. 215; J. Stella, “Pro-drugs as therapeutics”, Expert Opin. Ther. Patents, 14(3), 277-280, 2004; P. Ettmayer et al., “Lessons learned from marketed and investigational pro-drugs”, J. Med. Chem., 47, 2393-2404, 2004). In particular, pro-drugs are derivatives of the compounds of the invention in which functional groups carry additional substituents which may be cleaved under physiological conditions in vivo and thereby releasing the active principle of the compound (e. g., a pro-drug on being brought to a physiological pH or through an enzyme action is converted to the desired drug form). Pro-drugs of the compounds mentioned above are also within the scope of the present invention. Pro-drugs that are metabolised to compounds having formula (I), belong to the invention. In particular this relates to compounds with primary or secondary amino or hydroxy groups. Such compounds can be reacted with organic acids to yield compounds having formula (I) wherein an additional group is present which is easily removed after administration, for instance, but not limited to amidine, enamine, a Mannich base, a hydroxyl-methylene derivative, an O-(acyloxymethylene carbamate) derivative, carbamate, ester, amide or enaminone.

Any of the compounds of the present invention can be synthesized as pharmaceutically acceptable salts for incorporation into various pharmaceutical compositions. The term “pharmaceutically acceptable salts” refers to salt forms that are pharmacologically acceptable and substantially non-toxic to the subject being administered the compounds of the invention. Pharmaceutically acceptable salts of compounds of one of the formulae I through VI include conventional and stoichiometrical acid-addition salts or base-addition salts formed from suitable non-toxic organic or inorganic acids or inorganic bases.

Acid addition salts, for example, from compounds of the invention with a basic nitrogen atom are formed preferably with organic or inorganic acids. Suitable inorganic acids include, but are not limited to halogenic acids such as hydrochloric acid, sulfuric acid, or phosphoric acid. Suitable organic acids include, but are not limited to carboxylic, phosphonic, or sulfonic acids, for example acetic acid, propionic acid, glycolic acid, lactic acid, hydroxybutyric acid, malic acid, malei(ni)c acid, malonic acid, nicotinic acid, salicylic acid, fumaric acid, succinic acid, oxalic acid, phenylacetic acid, stearic acid, adipic acid, tartaric acid, citric acid, glutaric acid, 2- or 3-glycerophosphoric acid and other mineral and carboxylic acids well known to those skilled in the art. The salts are prepared by contacting the free base forms with a sufficient amount of the desired acid to produce a salt in the conventional manner.

Compounds of the invention containing acidic substituents may also form salts with inorganic or organic bases. Examples of suitable bases for salt formation include, but are not limited to, inorganic bases such as alkali or alkaline earth-metal (e.g., sodium, potassium, lithium, calcium, or magnesium) hydroxides, and those derived from ammonium hydroxides (e.g., a quaternary ammonium hydroxide such as tetramethyl ammonium hydroxide). Also contemplated are salts formed with pharmaceutical acceptable amines such as ammonia, alkyl amines, hydroxyalkylamines, N-methylglucamine, benzylamines, piperidines, pyridines, piperazines, and pyrrolidines and the like. Certain compounds will be acidic in nature, e. g. those compounds which possess a carboxyl or phenolic hydroxyl group. Salts of phenols can be made by heating acidic compounds with any of the above mentioned bases according to procedures well known to those skilled in the art.

As used herein, the term “composition” is intended to encompass a product comprising the specified ingredients in the specified amounts, as well as any product which results, directly or indirectly, from combination of the specified ingredients in the specified amounts.

The phrase “effective amount” as used herein, means an amount of a compound or composition which is sufficient enough to significantly and positively modify the symptoms and/or conditions to be treated (e. g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically acceptable excipient(s)/carrier(s) utilized, and like factors within the knowledge and expertise of the attending physician.

The term “mediate” means affect or influence. Thus, for example, conditions mediated by a progesterone receptor are those in which a progesterone receptor plays a role. Progesterone receptors are known to play a role in conditions including, for example, infertility, contraception, pregnancy maintenance and termination, female hormone deficiency, dysfunctional uterine bleeding, endometriosis, mood disorder, osteoporosis, and hormone-dependent cancers.

The term “progesterone receptor” as used herein always comprises the progesterone receptor alpha (PRα) and/or the progesterone receptor beta (PRβ) isoforms. Like other steroid hormone receptors, PR is expressed in two isoforms in certain organisms, including humans. Human PRα is a truncated form of human PRβ and lacks 164 amino acids at the N-terminus. Both isoforms are identical in the DNA-binding and ligand-binding domain and induce progestin-mediated gene transcription, but show a somehow different transactivation behavior (see e.g. WO 02/054064).

The terms “selective” and “selectivity” refer to compounds that display reactivity towards a particular receptor (e.g. a progesterone receptor) without displaying substantial cross-reactivity towards another receptor (e.g. glucocorticoid receptor, androgen receptor and/or estrogen receptor). Thus, for example, selective compounds of the present invention may display reactivity towards progesterone receptors without displaying substantial cross-reactivity towards other steroid hormone receptors. In one embodiment, a compound of the present invention has at least about 10 fold selectivity to the PR, at least about 50 fold selectivity to the PR, at least about 100 fold selectivity to the PR, at least about 250 fold selectivity to the PR, or at least about 500 fold selectivity to the desired target.

The following terms are used to describe various constituents of the chemical compositions useful in this invention. The terms are defined as follows, unless explicitly stated otherwise:

Any asymmetric carbon atoms may be present in the (R)-, (S)- or (R,S)-configuration, preferably in the (R)- or (S)-configuration, whichever is most active, unless the stereochemistry is explicitly depicted in the corresponding compound formula. Substituents at a double bond or a ring may be present in cis (═Z-) or trans (═E-) form, unless the stereochemistry is explicitly depicted in the corresponding compound formula.

The compounds of the invention have a defined stereochemistry within their steroidal core structure according to the commonly used definition of the configuration of retrosteroids (i.e. steroids with 9β, 10α conformation):

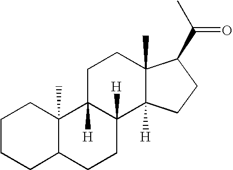

The stereochemistry within the retrosteroidal core structure is always shown in the corresponding compound formula and should not vary within the scope of the present invention, whereas the stereochemistry at the carbon atoms in the steroidal core carrying additional side chains and the stereochemistry of any asymmetric carbon atom within the side chains themselves is not fixed. Therefore, the terms “compounds of formula (I)” or “compounds of formula (II)” etc also comprise the stereoisomers of the depicted compounds, unless a particular stereochemistry is explicitly shown within the formula. The stereochemistry shown in the respective formula prevails over the general term “stereoisomers”.

The compounds of the present invention may contain further asymmetric centers on the molecule, e.g. a chiral carbon atom, depending upon the nature of the various substituents. In case of such an asymmetric centre, the compounds could thus be present in two optically active stereoisomeric forms or as a racemate. In certain instances, asymmetry may also be present due to restricted rotation about the central bond adjoining the two aromatic rings of the specified compounds. It is intended that all isomers (including enantiomers and diastereomers), either by nature of asymmetric centers or by restricted rotation as described above, as separated, pure or partially purified isomers or racemic mixtures thereof, be included within the ambit of the instant invention, unless a particular stereochemistry is explicitly depicted in the formula representing a respective compound.

The term “substituted” means that the specified group or moiety bears one or more substituents. Where any group may carry multiple substituents and a variety of possible substituents is provided, the substituents are independently selected and need not to be the same. The term “unsubstituted” means that the specified group bears no substituents. The term “optionally substituted” means that the specified group is unsubstituted or substituted by one or more substituents.

The term “halogen” refers to fluorine (F, Fluoro-), bromine (Br, Bromo-), chlorine (Cl, Chloro), and iodine (J, lodo-) atoms. The terms “dihalogen”, “trihalogen” and “perhalogen” refer to two, three and four substituents, respectively, each individually selected from the group consisting of fluorine, bromine, chlorine, and iodine atoms.

The term “hydroxyl” refers to the group —OH

The term “oxo” refers to the group ═O

The term “carbamoyl” refers to the group —CO—NH₂

The term “nitrile” or “cyano” refers to the group —CN.

The term “carbonyl” refers to the group —CHO.

The term “ketal” refers to a ketalized oxo group resulting from the reaction between two molecules of a monohydroxy aliphatic alcohol containing from 1 to 6 carbon atoms (e.g. Methanol, isopropanol, trichloroethanol, etc) and one molecule of an oxo group containing steroid, or resulting from the reaction between one molecule of a dihydroxy aliphatic alcohol containing from 2 to 6 carbon atoms (e.g. ethyleneglycol, 1,3-propanediol, etc.) and one molecule of an oxo group containing steroid.

For the purpose of the present invention, the carbon content of various hydrocarbon containing moieties is indicated by a prefix designating the minimum and maximum number of carbon atoms in the moiety, i.e., the prefix Cᵢ-Cⱼ defines the number of carbon atoms present from the integer “i” to the integer “j” inclusive. Thus C₁-C₄-alkyl refers to alkyl of 1-4 carbon atoms, inclusive, or methyl, ethyl, propyl, butyl and isomeric forms thereof.

The term “alkyl” stands for a hydrocarbon radical which may be linear, cyclic or branched, with single or multiple branching, whereby the alkyl group in general comprises 1 to 12 carbon atoms. In one embodiment, the term “alkyl” stands for a linear or branched (with single or multiple branching) alkyl chain of 1 to 4 carbon atoms, exemplified by the term (C₁-C₄)alkyl. The term (C₁-C₄)alkyl is further exemplified by such groups as methyl; ethyl; n-propyl; isopropyl; n-butyl; sec-butyl; isobutyl; and tert-butyl. The alkyl or (C₁-C₄)alkyl group may be partially unsaturated, forming such groups as, for example, vinyl, 1-propenyl, 2-propenyl (allyl), and butenyl. The term “alkyl” further comprises cycloalkyl groups, preferably cyclo(C₃-C₄)alkyl which refers to cyclopropyl or cyclobutyl, and isomeric forms thereof such as methylcyclopropyl. The cycloalkyl group may also be partly unsaturated. Furthermore, the term “alkyl” comprises a cycloalkyl-alkyl group comprising 4 to 12 carbon atoms, preferably “—(C₁-C₄)alkyl-cyclo(C₃-C₈)alkyl” which refers to a alkyl group of 1 to 4 carbon atoms substituted with a cyclo(C₃-C₈)alkyl group. Therefore, the term (C₁-C₄)alkyl also comprises a cyclopropylmethyl group.

The term “methylene” refers to —CH₂—and may be optionally substituted.

Halogenated alkyl, preferably halogenated (C₁-C₆)alkyl, are substituents in which the alkyl moieties (preferably (C₁-C₄)alkyl, most preferred methyl) are substituted either partially or in full with halogens, generally with chlorine and/or fluorine. Preferred examples of such substituents are trifluoromethyl, dichloromethyl, pentafluoroethyl, dichloropropyl, fluoromethyl and difluoromethyl.

The terms “aryl” or “Ar” refer to an aromatic carbocyclic group comprising 6 to 14, more preferably 6 to 10, carbon atoms and having at least one aromatic ring or multiple condensed rings in which at least one ring is aromatic. Preferably, aryl is phenyl, naphthyl, indanyl, indenyl, or 1,2,3,4-tetrahydro-naphthalen-1-yl; most preferred aryl is phenyl.

The term “heteroaryl” refers to an aromatic carbocyclic group of having a single 4 to 8 membered ring or multiple condensed rings comprising 6 to 14, more preferably 6 to 10, ring atoms and containing at least one heteroatom selected from N, O and S, within at least one ring, the number of N atoms being 0, 1, 2 or 3 and the number of O and S atoms each being 0 or 1; in which group at least one heterocyclic ring is aromatic. Examples of such groups include pyrrolyl, thienyl, furyl, imidazolyl, thiazolyl, isothiazolyl, oxazolyl, isoxazolyl, pyrazolyl, pyridinyl, pyrimidinyl, pyrazinyl, pyridazinyl, indolyl, quinolinyl, isoquinolinyl, benzothiazolyl, benzoimidazolyl, 1,3-dihydro-benzoimidazolyl, benzofuran, benzo[b]thiophene and the like. Preferably, heteroaryl is quinolinyl, furyl, benzoimidazolyl, pyridinyl, thienyl, indolyl, benzo[b]thiophene, pyridinyl, imidazolyl, pyrazolyl or thiazolyl. Most preferred heteroaryl refers to furyl or pyridyl.

In addition to the substituents explicitly exemplified here within, the aryl may be substituted by two groups which are attached to adjacent carbon atoms and are combined into a saturated or partly unsaturated cyclic 5, 6, 7, or 8 membered ring system, optionally containing 1, 2 or 3 heteroatoms selected from N, O or S, the number of N atoms being 0, 1, 2 or 3 and the number of O and S atoms each being 0, 1 or 2. Preferably, the two groups which are attached to adjacent carbon atoms, are combined into a saturated cyclic 5 or 6 membered ring system, optionally containing 1, 2 or 3 heteroatoms selected from N and O, the number of N atoms being 0, 1, 2 or-3 and the number of O atoms each being 0, 1 or 2. This cyclic ring system may optionally be further substituted by an oxo group. Preferred examples of such a substituted aryl groups are benzo[1,3]dioxol and 1,3-dihydro-benzoimidazol-2-one.

The term “aryl-(C₁-C₄)alkyl” refers to an (C₁-C₄)alkyl group substituted with an aryl group, wherein the aryl is phenyl, naphthyl, indanyl, indenyl, or 1,2,3,4-tetrahydro-naphthalen-1-yl, preferably aryl is phenyl or naphthyl, forming such groups as for example benzyl, phenethyl, phenylpropyl, phenylbutyl, naphthylmethyl or naphthylethyl. The alkyl chain may be partially unsaturated, such as a vinyl group. The aryl moiety may optionally be substituted as defined herein.

The statement is made that when two side chains (e.g. R¹² and R¹³) are found on a single N, e.g. as within the substituent —CO—NR¹²R¹³ or —NR¹²R¹³,they can be combined, including the N to which they are attached, into a heterocyclic ring of 4-, 5-, 6-, 7- or 8 atoms, which can be saturated, partly unsaturated or aromatic, and which can optionally contain 1, 2 or 3 additional heteroatoms selected from N, O or S, the number of N atoms being 0, 1, 2 or 3 and the number of O and S atoms each being 0, 1 or 2; and which ring can be part of a multiple condensed ring-system, in which some rings may be aromatic. Preferred examples of such heterocyclic ring systems, including the N to which the respective side chains are attached, are:

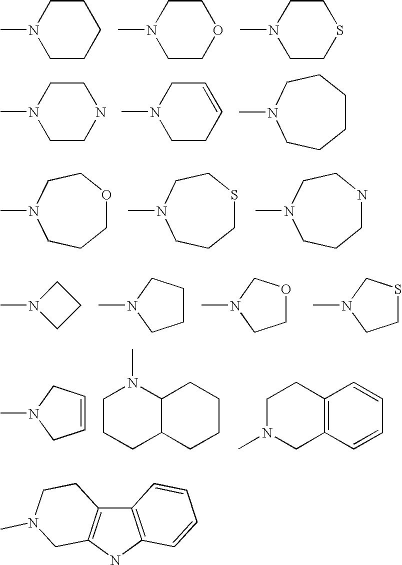

The aforementioned heterocyclic ring system can be optionally substituted by 1, 2 or 3 substituents, which can be attached to any carbon or nitrogen atom of the heterocyclic ring system. Preferred examples of substituted heterocyclic ring systems are:

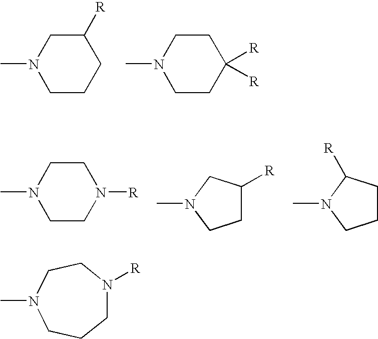

The optional 1, 2 or 3 independently selected substituents for the heterocyclic ring system may be chosen among —(C₁-C₄)alkyl, halogenated —(C₁-C₄)alkyl, halogen, hydroxyl, oxo, nitrile, aryl, aryl-(C₁-C₄)alkyl- and heteoaryl. Preferably, the heterocyclic ring system is optionally substituted with one or two substituents independently selected from the group of hydroxyl, oxo, (C₁-C₄)alkyl, aryl or aryl-(C₁-C₄)alkyl.

Compound Numbering (Nomenclature)

Furthermore, in an effort to maintain consistency in the naming of compounds of similar structure but differing substituents, the compounds described herein are named according to the following general guidelines. The numbering system for the location of substituents on such compounds is also provided.

The C-atoms of the steroidal core of the pregnane derivate are numbered according to the following general scheme:

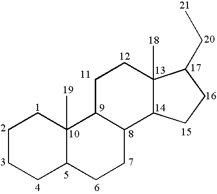

Dydrogesterone—((9β,10α-Pregna-4,6-diene-3,20-dione—has the following formula:

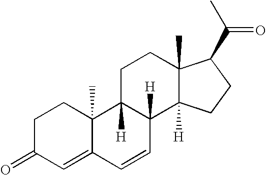

Retroprogesterone—((9β,10α)-Pregna-4-ene-3,20-dione—has the following formula:

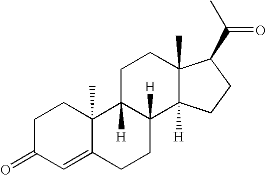

General structural formulas are typically designated with a number in Roman format I, II, III etc. Intermediates are indicated with the same numbers in Roman format as of the corresponding general formulas and a further letter or number, e.g. X-H for a particular derivative falling under compounds of the invention are designated No. 1, No. 2 etc.

Embodiments (Subclaims and Further Embodiments)

In a further embodiment, the present invention relates to compounds of the general formula (I) 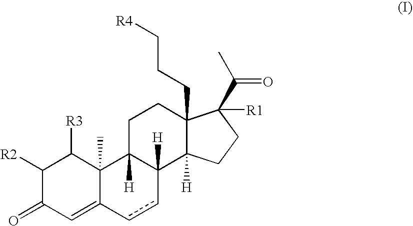
wherein
R1 is selected from hydrogen, —OH, —O—(C₁-C₄)alkyl, —O—CO—(C₁-C₄)alkyl, and —O—CO—O—(C₁-C₄)alkyl;
R2 and R3 are both hydrogen or together form a methylene group;
R4 is selected from —O—R⁶, heteroaryl and aryl.
   whereby the aryl group is optionally substituted with one or two substituents independently selected from the group consisting of
      —CHO; —CO—O—R⁹, —CO—NR¹²R¹³, —CH₂—O—R⁹; —CH═N—O—R¹⁴, —CH═N—O—CO—NHR¹², —CH═N—O—CO—R¹¹, —CH═N—O—CO—O—R¹⁴, —CH₂—NH—CO—NHR¹², —CH₂—NH—CO—R¹¹; and —CH₂—NH—CO—O—R¹⁴; -halogen and —O—R⁹,
   or which aryl is optionally substituted by two groups which are attached to adjacent carbon atoms and are combined into a saturated or partly unsaturated cyclic 5, 6 or 7-membered ring system, optionally containing 1 or 2 heteroatoms selected from N and O, the number of N atoms being 0, 1 or 2 and the number of O atoms being 0, 1 or 2;
R⁶, R⁹, R¹¹, R¹², R¹³ and R¹⁴ are independently selected from the group consisting of hydrogen, —(C₁-C₄)alkyl, and halogenated —(C₁-C₄)alkyl; or
R¹² and R¹³ form together with the nitrogen atom, where R¹² and R¹³ are attached, a heterocyclic 4-, 5-, 6-, 7- or 8-membered ring system, which is saturated, partly unsaturated, or aromatic; and which optionally contains 1, 2 or 3 additional heteroatoms selected from N, O or S, the number of additional N atoms being 0, 1, 2 or 3 and the number of O and S atoms each being 0, 1 or 2; and which ring is optionally part of a multiple condensed ring-system.

In one embodiment, R¹² and R¹³ form together with the nitrogen atom, where R¹² and R¹³ are attached, a heterocyclic 5-, 6- or 7-membered ring system, which is saturated or partly unsaturated; and which optionally contains 1 or 2 additional heteroatoms selected from N and O, the number of additional N atoms being 0, 1 or 2 and the number of 0 atoms being 0 or 1.

In another embodiment, the substituent R1 of the compounds of formula (I) is selected from hydrogen and —O—CO—O—(C₁-C₄)alkyl.

Furthermore, the invention preferably relates to compounds of general formula (I), wherein the substituents R2 and R3 both represent hydrogen.

In a further embodiment, the substituent R4 within compounds of formula (I) is selected from —OH, -phenyl, furyl and pyridyl,
whereby the phenyl group is optionally substituted with one or two, preferably one substituents in meta- and/or para-position independently selected from the group consisting of —CHO; —CO—O—R⁹, —CO—NR¹²R¹³, —CH₂—O—R⁹; —CH═N—O—R¹⁴, —CH═N—O—CO—NHR¹², -halogen and —O—R⁹,
or which phenyl is optionally substituted by two groups which are attached to adjacent carbon atoms and are combined into a saturated cyclic 5-, 6- or 7-membered ring system, optionally containing 1 or 2 O-atoms; and
wherein R⁹, R¹², R¹³ and R¹⁴ are independently selected from the group consisting of hydrogen, —(C₁-C₄)alkyl and halogenated —(C₁-C₄)alkyl; or
R¹² and R¹³ form together with the Nitrogen atom, where R¹² and R¹³ are attached, a saturated heterocyclic 5-, 6- or 7-membered ring system, which optionally contains 1 additional heteroatom selected from N and O.

Preferably, the compounds of the present invention have the following general formula (II)

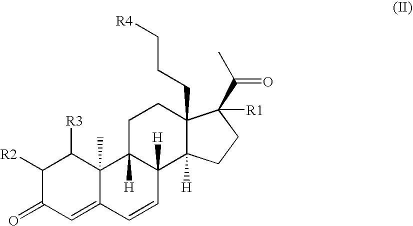

whereby the definition of the substituents is as described above here within for compounds of formula (I).

In a further embodiment, the present invention relates to compounds of the general formula (III) 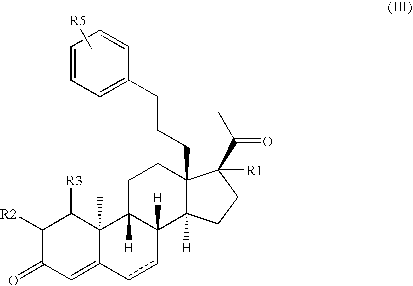
and in particular to compounds of general formula (V) 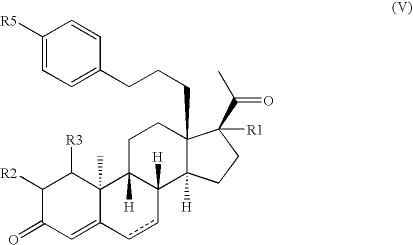
wherein
R1 is selected from hydrogen and —O—CO—O—(C₁-C₄)alkyl;
R2 and R3 both are hydrogen;
R5 is selected from the group consisting of —CHO; —CO—O—R⁹, —CO—NR¹²R¹³, —CH₂—O—R⁹; —CH═N—O—R¹⁴, —CH═N—O—CO—NHR¹², -halogen and —O—R⁹, and
R⁹, R¹², R¹³ and R¹⁴ are independently selected from the group consisting of hydrogen, —(C₁-C₄)alkyl and halogenated —(C₁-C₄)alkyl, or
R¹² and R¹³ form together with the nitrogen atom, where R¹² and R¹³ are attached, a heterocyclic 5-, 6- or 7-membered ring system, which is saturated or partly unsaturated; and which optionally contains 1 or 2 additional heteroatoms selected from N and O, the number of additional N atoms being 0, 1 or 2 and the number of O atoms being 0 or 1.

Preferably, the compounds of the present invention have the following general formula (IV) 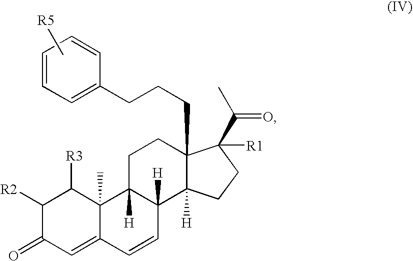
or have the following general formula (VI)

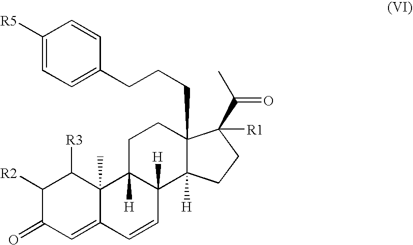

whereby the definition of the substituents is as described above here within for compounds of general formula (III) and for compounds of general formula (V).

Representative PR modulator compounds (i.e., agonists, partial agonists and antagonists) according to the present invention include
18-[2-(4-Oximino-formylphenyl)-ethyl]-((9β,10α)-pregna-4-ene-3,20-dione (No. 1)
18-[2-(4-Oximino-formylphenyl)-ethyl]-((9β,10α)-pregna-4,6-diene-3,20-dione (No. 2)
18-[2-(4-Oximino-formylphenyl)-ethyl]-3,20-dioxo-((9β,10α)-pregna-4,6-diene-17-yl-carbonic acid ethyl ester (No. 3)
18-[2-(4-N-Ethylcarbamoyl-oximino-formylphenyl)-ethyl]-((9β,10α)-pregna-4-ene-3,20-dione (No. 4)
18-[2-(4-N-Ethylcarbamoyl-oximino-formylphenyl)-ethyl]-((9β,10α)-pregna-4,6-diene-3,20-dione (No. 5)
18-[2-(4-N-Ethylcarbamoyl-oximino-formylphenyl)-ethyl]-3,20-dioxo-((9β,10α)-pregna-4,6-diene-17-yl-carbonic acid ethyl ester (No. 6)
18-[2-(4-Hydroxymethyl-phenyl)-ethyl]-(9β,10α)-pregna-4-ene-3,20-dione (No. 7)
18-(2-[4-Hydroxymethyl-phenyl]-ethyl)-3,20-dioxo-(9β,10α)-pregna-4-ene-17-yl-carbonic acid ethyl ester (No. 8)
18-[2-(4-Formyl-phenyl)-ethyl]-(9β,10α)-pregna-4-ene-3,20-dione (No. 9)
18-[2-(4-Formyl-phenyl)-ethyl]-(9β,10α)-pregna-4,6-diene-3,20-dione (No .10)
18-[2-(4-Formyl-phenyl)-ethyl]-3,20-dioxo-(9β,10α)-pregna-4-ene-17-yl-carbonic acid ethyl ester (No. 11)
18-[2-(4-Formyl-phenyl)-ethyl]-3,20-dioxo-(9β,10α)-pregna-4,6-diene-17-yl-carbonic acid ethyl ester (No. 12)
18-[2-(4-Formamido-phenyl)-ethyl]-(9β,10α)-pregna-4,6-diene-3,20-dione (No. 13)
18-[2-(4-Formic acid-phenyl)-ethyl]-(9β,10α)-pregna-4-ene-3,20-dione (No. 14)
18-[2-(4-Formic acid-phenyl)-ethyl]-(9β,10α)-pregna-4,6-diene-3,20-dione (No. 15)
18-[2-Phenyl)-ethyl]-(9β,10α)-pregna-4-ene-3,20-dione (No. 16)
18-[2-Phenyl)-ethyl]-(9β,10α)-pregna-4,6-diene-3,20-dione (No. 17)
18-[2-benzo[1,3]dioxol-5-yl-ethyl]-(9β,10α)-pregna-4-ene-3,20-dione (No. 18)
18-[2-benzo[1,3]dioxol-5-yl-ethyl]-(9β,10α)-pregna-4,6-diene-3,20-dione (No. 19)
18-[2-(3,4-Difluoro-phenyl)-ethyl]-(9β,10α)-pregna-4-ene-3,20-dione (No. 20),
18-[2-(3,4-Difluoro-phenyl)-ethyl]-(9β,10α)-pregna-4,6-diene-3,20-dione (No. 21),
18-[2-Pyridin-3-yl-ethyl]-(9β,10α)-pregna-4-ene-3,20-dione (No. 22),
18-[2-Pyridin-3-yl-ethyl]-(9β,10α)-pregna-4,6-diene-3,20-dione (No. 23),
18-[2-(3-Methoxy-phenyl)-ethyl]-(9β,10α)-pregna-4-ene-3,20-dione (No. 24),
18-[2-(3-Methoxy-phenyl)-ethyl]-(9β,10α)-pregna-4,6-diene-3,20-dione (No. 25),
18-[2-(4-Methoxy-phenyl)-ethyl]-(9β,10α)-pregna-4-ene-3,20-dione (No. 26),
18-[2-(4-Methoxy-phenyl)-ethyl]-(9β,10α)-pregna-4,6-diene-3,20-dione (No. 27),
18-[2-(3,5-Dimethoxy-phenyl)-ethyl]-(9β,10α)-pregna-4-ene-3,20-dione (No. 28),
18-[2-(3,5-Dimethoxy-phenyl)-ethyl]-(9β,10α)-pregna-4,6-diene-3,20-dione (No. 29),
18-[2-(3-Trifluoro-methoxy-phenyl)-ethyl]-(9β,10α)-pregna-4-ene-3,20-dione (No. 30),
18-[2-(3-Trifluoro-methoxy-phenyl)-ethyl]-(9β,10α)-pregna-4,6-diene-3,20-dione (No. 31),
18-{2-[4-(morpholine-4-carbonyl)-phenyl]-ethyl}-(9β,10α)-pregna-4-ene-3,20-dione (No. 32), and
18-{2-[4-(morpholine-4-carbonyl)-phenyl]-ethyl}-(9β,10α)-pregna-4,6-diene-3,20-dione (No. 33).
   Administration Forms (Pharmaceutical Formulations)

The method of the invention is primarily intended for treatment in a mammal, preferably in humans and other primates, of diseases, disorders or conditions mediated by progesterone receptors, or of diseases, disorders or conditions that can be treated via modulation of those receptors. In particular, the invention concerns the therapeutic use of said novel retrosteroidal derivatives in the treatment or prevention of benign gynecological disorders, especially endometriosis and uterine fibroids, in hormonal female contraception or in hormone replacement therapy

The compounds may be administered orally, dermally, parenterally, by injection, by pulmonal or nasal delivery, or sublingually, or by topical administration, i.e. rectally, vaginally, or within the intrauterine cavity, in dosage unit formulations. The term “administered by injection” includes intravenous, intraarticular, intramuscular (e.g. by depot injection where the active compounds are released slowly into the blood from the depot and carried from there to the target organs), intraperitoneal, intradermal, subcutaneous, and intrathecal injections, as well as use of infusion techniques. Dermal administration may include topical application or transdermal administration. One or more compounds may be present in association with one or more non-toxic pharmaceutically acceptable auxiliaries such as excipients, adjuvants (e.g. buffers), carriers, inert solid diluents, suspensing agents, preservatives, fillers, stabilizers, anti-oxidants, food additives, bioavailability enhancers, coating materials, granulating and disintegrating agents, binding agents etc., and, if desired, other active ingredients.

The pharmaceutical composition may be formulated for example as immediate release, sustained release, pulsatile release, two or more step release, depot or other kind of release formulations.

The manufacture of the pharmaceutical compositions according to the invention may be performed according to methods known in the art and will be explained in further detail below. Commonly known and used pharmaceutically acceptable auxiliaries as well as further suitable diluents, flavorings, sweetening agents, coloring agents etc. may be used, depending on the intended mode of administration as well as particular characteristics of the active compound to be used, such as solubility, bioavailability etc. Suitable auxiliaries and further ingredients may be such as recommended for pharmacy, cosmetics and related fields and which preferably are listed in the European Pharmacopoeia, FDA approved or cited in the “GRAS” list (FDA List of food additives that are ‘generally recognized as safe’ (GRAS)).

One mode of application of the compounds of general formula (I) or of pharmaceutical compositions comprising one or more of said compounds is oral application, e. g., by tablets, pills, dragees, hard and soft gel capsules, granules, pellets, aqueous, lipid, oily or other solutions, emulsions such as oil-in-water emulsions, liposomes, aqueous or oily suspensions, syrups, elixiers, solid emulsions, solid dispersions or dispersible powders. For the preparation of pharmaceutical compositions for oral administration, the compounds suitable for the purposes of the present invention as defined above can be admixed with commonly known and used adjuvants and excipients such as for example, gum arabic, talcum, starch, sugars (such as, e. g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e. g., ethereal oils), solubility enhancers (e. g., benzyl benzoate or benzyl alcohol) or bioavailability enhancers (e.g. Gelucire™). In the pharmaceutical composition, the active ingredients may also be dispersed in a microparticle, e. g. a nanoparticulate, composition.

For parenteral administration, the active agents can be dissolved or suspended in a physiologically acceptable diluent, such as, e. g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally spoken, for parenteral administration the active agent can be in the form of an aqueous, lipid, oily or other kind of solution or suspension or even administered in the form of liposomes or nano-suspensions.

Transdermal application can be accomplished by suitable patches, as generally known in the art, specifically designed for the transdermal delivery of active agents, optionally in the presence of specific permeability enhancers. Furthermore, also emulsions, ointments, pastes, creams or gels may be used for transdermal delivery.

Another suitable mode of administration is via intravaginal devices (e. g. vaginal rings) or intrauterine systems (IUS) and intrauterine devices (IUD), respectively, containing reservoirs for controlled release of active agents over extended periods of time. Such IUS or IUDs (as, e.g., MIRENA™) is introduced into the uterine cavity where it continuously releases defined amounts of hormone for up to 5 years (or until the system is removed).

For rectal or vaginal administration of the drug the compounds may also be administered in the form of suppositories. These compositions can be prepared by mixing the drug with a suitable non-irritating excipient which is solid at ordinary temperatures but liquid at the rectal or vaginal temperature and will therefore melt in the rectum or vagina to release the drug.

A further drug formulation is a formulation intended for the topical, local and/or regional administration of the compound to the reproductive organs, in particular to a body region selected from the group consisting of the uterus, fallopian tubes, peritoneal space, pelvic cul-de-sac, ovaries, and urinogenital tract, in amounts effective to treat various conditions, particularly local diseases of the female reproductive system, such as pelvic, uterine, cervical and vaginal diseases, as described e.g. within EP 0977555 A1, U.S. Pat. No. 5,993,856, U.S. Pat. No. 6,652,874, or U.S. Pat. No. 6,416,778. The formulation comprises drug particles, preferably in the form of a micro- or nano-particles, suitable for regional administration of an effective amount of drug, wherein the effective amount is a dosage which results in low serum drug levels and reduced side effects as compared to systemic administration of the drug. In particular, the formulation comprises a carrier promoting quick uptake of the drug into the blood stream, a carrier manipulating release of drug, or a carrier promoting adhesion of the drug selected from the group consisting of a liquid suspension or dispersion, a hydrogel suspension or dispersion, a topical ointment, a cream, a lotion, and a foam.

Another mode of administration is by implantation of a depot implant comprising an inert carrier material, such as biologically degradable polymers or synthetic silicones such as e. g. silicone rubber. Such implants are designed to release the active agent in a controlled manner over an extended period of time (e. g., 3 to 5 years).

It will be appreciated by those skilled in the art that the particular method of administration will depend on a variety of factors, all of which are considered routinely when administering therapeutics. It will also be understood, however, that the actual dosages of the agents of this invention for any given patient will depend upon a variety of factors, including, but not limited to the activity of the specific compound employed, the particular composition formulated, the mode of administration, time of administration, route of administration and the particular site, host, and disease being treated, and furthermore the age of the patient, the body weight of the patient, the general health of the patient, the gender of the patient, the diet of the patient, rate of excretion, drug combinations, and the severity of the condition undergoing therapy. It will be further appreciated by one skilled in the art that the optimal course of treatment, i.e., the mode of treatment and the daily number of doses of a compound of Formula I or a pharmaceutically acceptable salt thereof given for a defined number of days, can be ascertained by those skilled in the art using conventional treatment tests. Optimal dosages for a given set of conditions may be ascertained by those skilled in the art using conventional dosage-determination tests in view of the experimental data for a given compound. For oral administration, an exemplary daily dose generally employed will be from about 0.001 μg/kg to about 10 mg/kg of total body weight, whereby courses of treatment may be repeated at appropriate time intervals. Administration of pro-drugs may be dosed at weight levels that are chemically equivalent to the weight levels of the fully active compounds. The daily dosage for parenteral administration will generally be from about 0.001 μg/kg to about 10 mg/kg of total body weight. A daily rectal dosage regimen will generally be from about 0.001 μg/kg to about 20 mg/kg of total body weight. A daily vaginal dosage regimen will generally be from about 0.001 μg/kg to about 10 mg/kg of total body weight. The daily topical dosage regimen will generally be from about 0.01 μg to about 10 mg administered between one to four times daily. The transdermal concentration will generally be that required to maintain a daily dose of from 0.001 μg/kg to 10 mg/kg of total body weight. The total dosage of administration forms releasing the drug compound over a prolonged period of time, i.e. from about several weeks to some years, depends on the time of administration, on the kind of device (intravaginal devices, intrauterine systems, intrauterine devices, implants etc.) and on the kind of release behaviour of the particular device. In general, the daily released dose of active compound will be from about 0.001 μg/kg to about 1 mg/kg of total body weight. Since the devices often only need to achieve a certain local and/or regional concentration of active compound, the daily released dosage can be lower in comparison to e.g. oral administration.

Abbreviations and Acronyms

As employed herein, the following terms have the indicated meanings.

abs absolute

ACN acetonitrile

AP alkaline phosphatase

aq aqueous

AR androgen receptor

conc. concentrated

d day(s)

DCC Dicyclohexylcarbodiimide

DCM dichloromethane CH₂Cl₂

DDQ 2,3-dichloro-5,6-dicyano-p-benzoquinone

DEE Diethyl ether

DHP 3,4-dihydro-[2H]-pyran

DIBAH diisobutyl-aluminium-hydride

DIPEA N,N-diisopropylethylamine

DMAP 4-(N,N-dimethylamino)-pyridine

DMF N,N-dimethylformamide

DMSO dimethylsulfoxide

EDCI 1-(3-dimethylaminopropyl)-3-ethylcarbodiimide

EDCI.HCl 1-(3-dimethylaminopropyl)-3-ethylcarbodiimide hydrochloride

ER estrogen receptor

EtOAc ethyl acetate

GR glucocorticoid receptor

GRAS ‘generally recognized as safe’

h hour(s)

HOBT Hydroxybenzotriazole-Hydrate

IUD intrauterine device

LAH lithium aluminium hydride

LTA lead tetra acetate

MeOH methanol

min minute(s)

MTBE methyl tertiary butyl ether

NMMO N-methylmorpholine-N-oxide

NMR nuclear magnetic resonance

PCC pyridinium chlorochromate

PG protective group

PR progesterone receptor

pTosOH para-toluene sulfonic acid

RT room temperature

SPRM selective progesterone receptor modulator

TBDPS tert-Butyldiphenylsilyl

TBME tert-butyl methyl ether

TEA triethylamine

TEMPO 2,2,6,6-tetramethyl-1-piperidinyloxy, free radical

THF tetrahydrofuran

THP tetrahydropyran

TLC thin-layer chromatography

TMOF trimethyl orthoformate

TMSCI trimethylsilylchloride

TPP triphenylphosphine

General Preparative Methods

The compounds of the present invention may be prepared from 9β,10α-steroids by use of known chemical reactions and procedures. Nevertheless, the following general preparative methods are presented to aid the reader in synthesizing the SPRM compounds of the present invention, with specific details provided below in the experimental section to illustrate working examples.

All variable groups of these methods are as described in the generic description if they are not specifically defined below.

It is recognized that compounds of the invention with each claimed optional functional group may not be prepared by each of the below-listed methods. Within the scope of each method, optional substituents may appear on reagents or intermediates which may act as protecting or otherwise non-participating groups. Utilizing methods well known to those skilled in the art, these groups are introduced and/or removed during the course of the synthetic schemes which provide the compounds of the present invention.

Flow Diagrams The sequence of steps for the general schemes to synthesize the compounds of the present invention is shown below. In each of the Schemes the R groups (e. g., R1, R2, etc.) correspond to the specific substitution patterns noted in the Description and the Examples. However, it will be understood by those skilled in the art that other functionalities disclosed herein at the indicated positions of compounds of formulae I, II and III also comprise potential substituents for the analogous positions on the structures within the Schemes.

Intermediate: 18-formyl-(9β,10α)-pregna-5-ene-3,20-diketal of formula XVI

The synthesis of the first key intermediate 18-formyl-(9β,10α)-pregna-5-ene-3,20-diketal of formula XVI, optionally substituted in C1-C2 position with a methylene group,

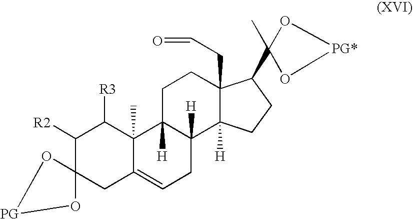

wherein PG and PG* represent conventional protective groups for the keto function of the steroidal core (e.g. forming a dialkyl or a cyclic ketal derivative), can be performed according to the procedures disclosed in U.S. Pat. No. 3,555,053 and as described by van Moorselaar and Halkes [1969], and as displayed in the following general SCHEME I.

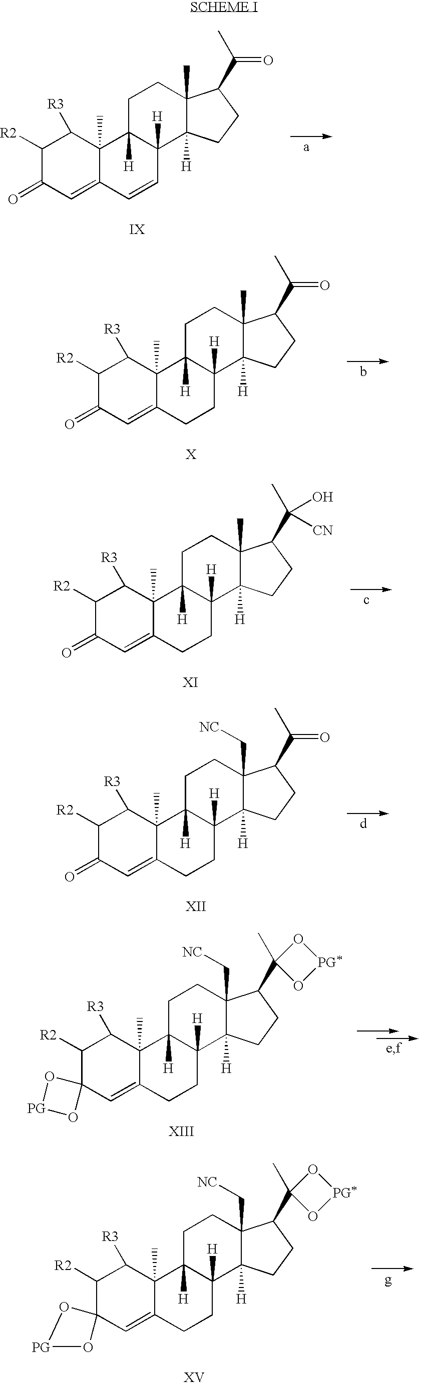

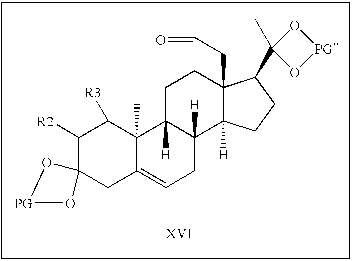

Commercially available Dydrogesterone (9β,10α-pregna-4,6-diene-3,20-dione), which is optionally substituted in the 1,2 position with a methylene group is used as starting material. The introduction of the 1,2-methylene group might be performed according to the well known procedures as described by Halkes et al [1972] and within U.S. Pat. No. 3,937,700 for 17α-Hydroxy-9β,10α-pregna-4,6-diene-3,20-dione by dehydrogenation and subsequent reaction with Dimethylsulfoxonium methylide. The optionally 1,2 methylene substituted 9β,10α-pregna-4,6-diene-3,20-dione of general formula IX is then converted to the corresponding 9β,10α-pregna-4-ene-3,20-dione (9β,10α-progesterone) of general formula X under reducing conditions (step a). In step b, the compound of general formula X is reacted with HCN to produce the corresponding 20-cyano-20-hydroxy compound of formula XI, followed by irradiation in the presence of iodine and lead-tetra-acetate to yield the 18-cyano derivative of the general formula XII (step c). Then, the two oxo-groups of said 18-cyano derivative are protected by ketalization, preferably with a dihydroxy alcohol, in the presence of a catalyst producing the 18-cyano-3,20-diketal derivative of general formula XIII (step d), which is then transformed into the corresponding Δ5-18-cyano-3,20-ketalized dione of general formula XV by isomerization, partial deketalization and chromatographic separation of the resulting mixture of the Δ5-diketal and the Δ4-20-monoketal derivatives (steps e and f). Subsequently, the Δ5-18-cyano-3,20-diketal of general formula XV is treated with a reducing agent such as diisobutyl-aluminium-hydride (DIBAH) to gave an aidimine intermediate, which is hydrolyzed to the desired 18-formyl-(9β,10α)-pregna-5-ene-3,20-diketal compound of general formula XVI (step g).

Derivatisation of the Carbonyl Function of the 18-formyl-(9β,10α)-pregna-5-ene-3,20-diketal of formula XVI

The aim of the next reaction steps is the derivatisation of the formyl group in C18 position of the retrosteroidal core using a Wittig addition reaction (step h) as displayed in the following general SCHEME II:

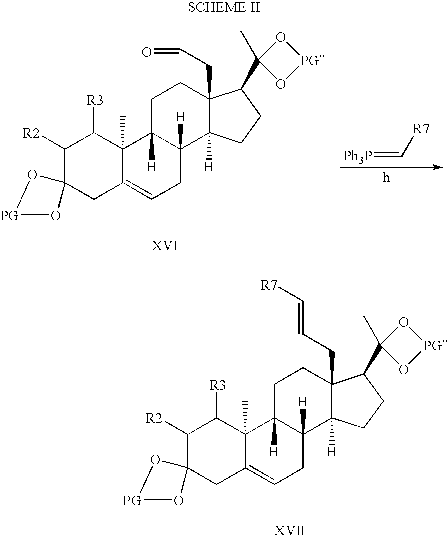

wherein PG and PG* represent conventional protection groups for the keto function of the steroidal core (e.g. forming a dialkyl or a cyclic ketal derivative), R², R³ have the aforesaid meanings, and wherein R⁷ represents hydrogen or a heteroaryl or aryl residue. The heteroaryl or aryl residue is optionally substituted in the heteroaryl or aryl group with one or two substituents independently selected from the group consisting of —CH₂—O—PG**; —CH₂—O—R⁹′, —CO—O—PG**, —CO—O—R⁹′, —CO—NR¹²′R¹³′, —CN, -halogen, —O—PG**, —O—R⁹′, —N(PG**)₂, —NPG**R¹⁰, —NR¹²′R¹³′, —(C₁-C₄)alkyl, and halogenated —(C₁-C₄)alkyl, whereby PG** represents a conventional protection group for the hydroxyl or amine function, and whereby R⁹′, R¹²′ and R¹³′, represent —(C₁-C₄)alkyl or halogenated —(C₁-C₄)alkyl, or R¹²′ and R¹³′ form together with the nitrogen atom, where they are attached, a heterocyclic 4-, 5-, 6-, 7- or 8-membered ring system, which is saturated, partly unsaturated, or aromatic; and which optionally contains 1, 2 or 3 additional heteroatoms selected from N, O or S, the number of additional N atoms being 0, 1, 2 or 3 and the number of O and S atoms each being 0, 1 or 2; and which ring is optionally part of a multiple condensed ring-system. Alternatively, the aryl moiety of R⁷ is optionally substituted by two groups which are attached to adjacent carbon atoms and are combined into a saturated or partly unsaturated cyclic 5, 6, 7, or 8 membered ring system, optionally containing 1, 2 or 3 heteroatoms selected from N, O and S, the number of N atoms being 0, 1, 2 or 3 and the number of O and S atoms each being 0, 1 or 2.

Suitable protective groups PG** or other protective groups mentioned in this application are known in the art and can routinely be selected by a person skilled in the art; more information on addition and subsequent removal of protective groups in organic synthesis can be found in: T. W. Greene & P. G. M. Wuts “Protective groups in Organic Synthesis” John Wiley & Sons, in its latest edition.

The Wittig reagent—the Ph₃P═CH—R⁷ Phorphoran (or Phosphonium-Ylid)—is freshly prepared from the corresponding Triphenylphosphonium halogenide salt (Ph₃P—CH₂—R⁷)Hal by contact with a strong base such as Phenyllithium or Butyllithium. The corresponding Triphenylphosphonium halogenide salt (Ph₃P—CH₂—R⁷)Hal is commercially available or can be synthesized by methods known to the skilled artisan, e.g. starting from the corresponding commercially available halogenide derivative by reaction with Triphenylphosphine (TPP). If necessary the halogenide derivative can be prepared from the corresponding hydroxyl derivative. The freshly prepared Wittig reagent is then reacted with the carbonyl function of the 18-formyl-(9β,10α)-pregna-5-ene-3,20-diketal of formula XVI to produce the corresponding unsaturated addition product of general formula XVII (step h).

The further reaction steps are different depending on the nature of R⁷.

A: R⁷ Represents Hydrogen

Synthesis of Compounds of General Formula I wherein R⁴ Represents —O—R⁶:

If R⁷ represents hydrogen, the next reaction steps starting from the 18-vinyl-(9β,10α)-pregna-5-ene-3,20-diketal of general formula XVII are performed to produce the derivatives of general formula I wherein R⁴ represents —O—R⁶, and R⁶ is hydrogen, —(C₁-C₄)alkyl, or halogenated —(C₁-C₄)alkyl.

Thus, the compounds of the invention of general formula XIX and XXII

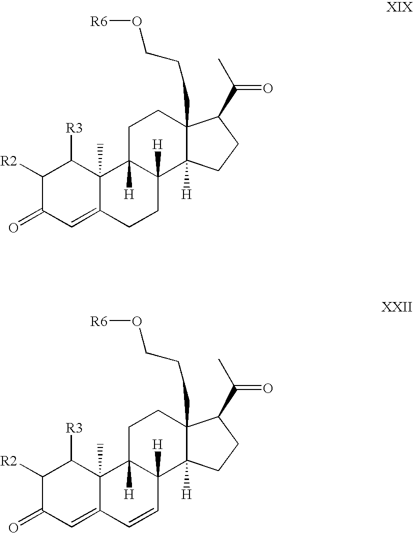

are obtained according to the reactions as displayed in the following general SCHEME III:

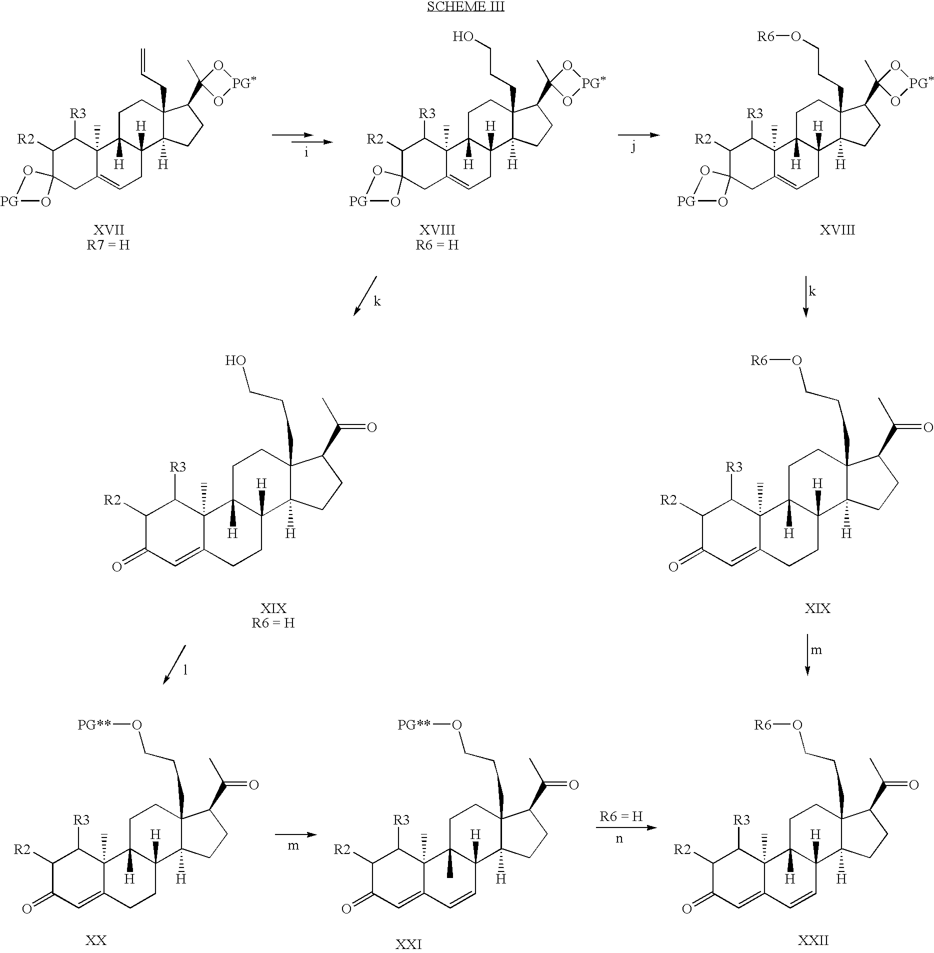

wherein PG and PG* represent conventional protective groups for the keto function of the steroidal core (e.g. forming a dialkyl or a cyclic ketal derivative), PG** represents a conventional protective group for the hydroxyl function (e.g. an acyl group), and R², R³, and R⁶ have the meanings given above. The optionally 1,2-methylene substituted 18-vinyl-(9β,10α)-pregna-5-ene-3,20-diketal of general formula XVII is converted into the corresponding 18-(2-Hydroxyethyl)-(9β,10α)-pregna-5-ene-3,20-diketal of general formula XVIII with R6 representing hydrogen by hydroboration and subsequent oxidation to deliver the alcohol (step i). In an optional step j, the alcohol of general formula XVIII with R6 representin hydrogen is reacted with an appropriate (C₁-C₄)-alkyl-halogenide to produce the corresponding 18-(2-Alkoxyethyl)-(9β,10α)-pregna-5-ene-3,20-diketal of general formula XVIII. The obtained derivatives of general formula XVIII (with R6=H) or XVIII are then subjected to deketalization delivering the 18-(2-Alkoxyethyl)- or 18-(2-Hydroxyethyl)-(9β,10α)-pregna-4-ene-3,20-dione of general formula XIX and XIX (with R6=H), respectively (step k). The obtained compounds do fall under the scope of the compounds of general formula I and belong to the compounds of the invention. In order to reintroduce the second double bond in 6,7-position of the steroid core, the free hydroxyl group of compound XIX (with R6=H) has to be protected first (step l). The dehydrogenation reaction of step m delivers the corresponding (9β,10α)-pregna-4,6-diene-3,20-dione derivatives of general formula XXII, optionally after deprotection of the free hydroxyl group (step n).

A: R⁷ Represents Optionally Substituted Aryl or Heteroaryl

Synthesis of Compounds of General Formula I wherein R⁴ Represents Optionally Substituted Aryl or Heteroaryl:

If R⁷ represents an optionally substituted aryl or heteroaryl group, the next reaction step starting from intermediate of general formula XVII is the reduction of the unsaturated side chain to produce the corresponding 18-(R⁷-substituted)-ethyl-(9β,10α)-pregna-5-ene-3,20-diketal of general formula XXIII according to the following general SCHEME IV:

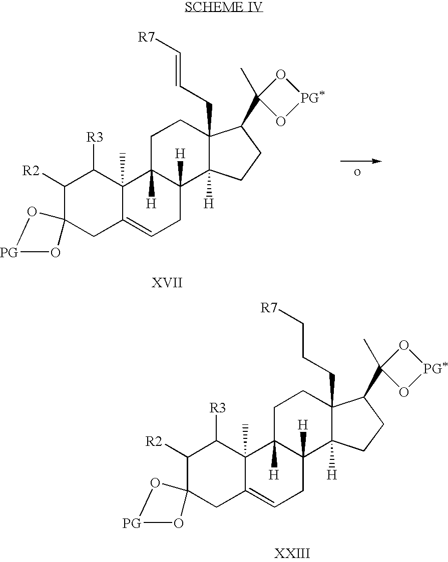

wherein PG, PG* and R⁷ have the same meaning as defined above in general SCHEME II, but R⁷ cannot represent hydrogen. The reduction is preferably carried out by catalytic hydrogenation (step o).

Starting from the 18-(R⁷-substituted)-ethyl-(9β,10α)-pregna-5-ene-3,20-diketal of general formula XXIII, the following compounds of general formula XXVI and XXVII can be prepared, which fall under the scope of general formula I and represent compounds of the invention which show progesterone receptor modulating properties

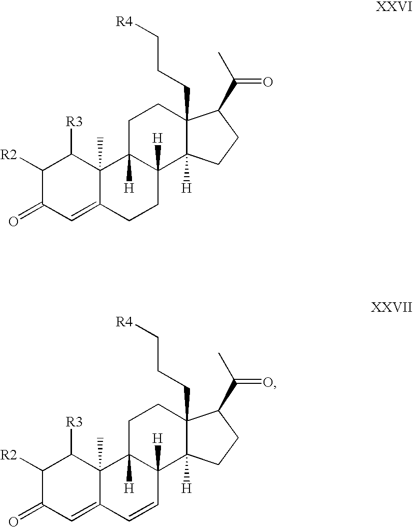

wherein R², R³ have the aforementioned meanings and R⁴ represents optionally substituted aryl or heteroaryl as defined here within for the compounds of the invention according to general formula (I). These compounds of general formula XXVI and XXVII are produced by a series of different reaction steps comprising at least one deketalization step (step p) and a further dehydrogenation step (step q) in case the 4,6-diene is the desired product, as displayed within general SCHEME V. Depending on the starting intermediate of general formula XXIII, in particular depending on the kind of substituent of the aryl or heteroaryl moiety in R⁷, further reactions have to be performed in order to obtain the desired substituent R⁴. These transformation reactions are described in more detail below, whereby the deketalization reaction (step p) and the dehydrogenation (step q) may be performed when it appears most appropriate in the overall reaction scheme, i.e. also after modification of R⁷ and/or independently from each other.

The following SCHEME V shows the transformation of general compound XXIII to compounds XXIV by deketalization (step p) and XXV by dehydrogenation (step q), which compounds correspond to desired compounds XXVI and XXVII, in case that R⁷ already represents the desired residue R⁴ or can be transformed into R⁴ by one or more additional reaction steps.

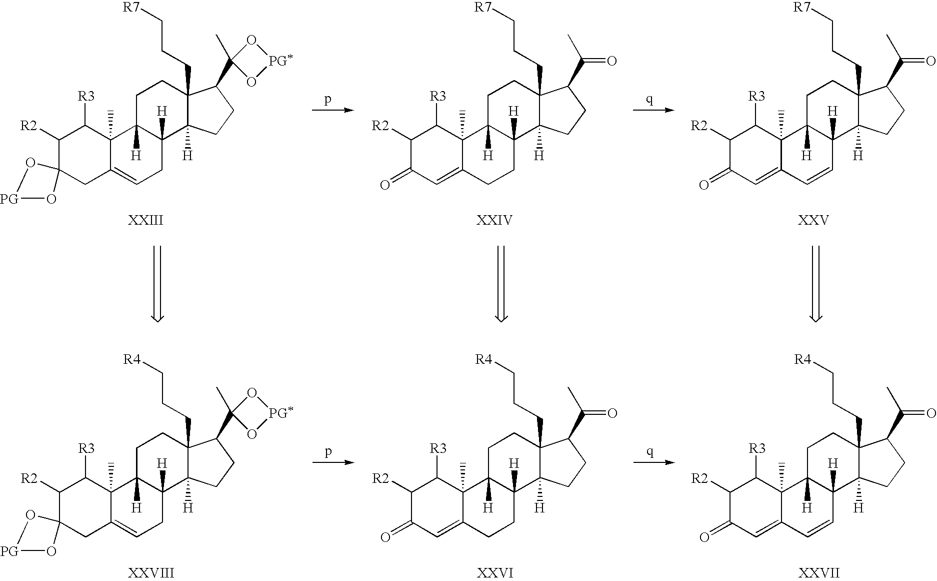

wherein R², R³, PG, PG*, R⁷ and R⁴ have the meanings as given above.

If R⁷ already represents the desired residue R⁴ or can be easily transformed into R⁴ (i.e. when R⁷ represents a heteroaryl or aryl residue, optionally substituted in the heteroaryl or aryl group with one or two substituents independently selected from the group consisting of —CH₂—O—R⁹′, —CO—O—R⁹′, —CO—NR¹²′R¹³′, —CN, -halogen, —O—R⁹′, —NR¹²′R¹³′, —(C₁-C₄)alkyl, and halogenated —(C₁-C₄)alkyl, whereby R⁹′, R¹²′ and R¹³′ represent —(C₁-C₄)alkyl or halogenated —(C₁-C₄)alkyl, or R¹²′ and R¹³′ form together with the nitrogen atom, where they are attached, a heterocyclic 4-, 5-, 6-, 7- or 8-membered ring system, which is saturated, partly unsaturated, or aromatic; and which optionally contains 1, 2 or 3 additional heteroatoms selected from N, O or S, the number of additional N atoms being 0, 1, 2 or 3 and the number of O and S atoms each being 0, 1 or 2; and which ring is optionally part of a multiple condensed ring-system; or wherein the aryl moiety of R⁷ is optionally substituted by two groups which are attached to adjacent carbon atoms and are combined into a saturated or partly unsaturated cyclic 5, 6, 7, or 8-membered ring system, optionally containing 1, 2 or 3 heteroatoms selected from N, O and S, the number of N atoms being 0, 1, 2 or 3 and the number of O and S atoms each being 0, 1 or 2), then the 18-(R⁷-substituted)-ethyl-(9β,10α)-pregna-5-ene-3,20-diketal intermediate of general formula XXIII is subjected to the deketalization step p producing the compound XXIV, optionally directly followed by the oxidation step q producing the compound XXV, thereby delivering the desired compounds of general formula XXVI and XXVII.

If R⁷ represents a heteroaryl or aryl residue, optionally substituted in the heteroaryl or aryl group with one or two substituents independently selected from the group consisting —CH₂—O—PG**; —CO—O—PG**, —O—PG**, —N(PG**)R¹⁰, —N(PG**)₂, or —CN, and whereby one substituent may also be selected from the group consisting of —CH₂—O—R⁹′, —CO—O—R⁹′, —CO—NR¹²′R¹³′, -halogen, —O—R⁹′, —NR¹²′R¹³′, —(C₁-C₄)alkyl, and halogenated —(C₁-C₄)alkyl, necessary modifications of R⁷ and its substituents in the aryl or heteroaryl moiety, respectively, typically start with a deprotection step by removing the PG** group from the above cited substituents, thereby leading to a functional group —CH₂—OH; —CO—OH, —OH, —NHR¹⁰, and —NH₂, respectively. In case of a substituent —CN, optional further modifications include the reduction the cyano group to a —CH₂—NH₂ substituent, using a conventional reducing agent such as lithium aluminium hydride in THF, sodium borohydride in an alcoholic solvent or by catalytic reduction with e.g. Raney Nickel.

This modification step of the substituent in the aryl or heteroaryl moiety of R⁷ results in derivatives of the compounds of general formula XXIII, XXIV or XXV carrying a modified R⁷-ethyl residue, called R⁷¹-ethyl residue, in the C18 position of the retrosteroidal core, or directly delivers compounds of general formula XXVIII, XXVI or XXVI in case that the modified R⁷ residue (i.e. the R⁷¹ residue) already represents the desired residue R⁴. Accordingly, the modified residue R⁷¹ preferably represents an aryl or heteroaryl group, optionally substituted with one or two substituents independently selected from the group consisting of —CH₂—OH; —COOH, —OH, —NHR¹⁰, —NH₂, and —CH₂—NH₂, and a substituent of the group as listed above for the residue R⁷.

As already stated above, the deketalization of the intermediate 18-(2-R^{7/71/4}′-substituted-ethyl)-(9β,10α)-pregna-5-ene-3,20-diketal derivatives to produce the corresponding (9β,10α)-pregna-4-ene-3,20-dione (step p), optionally followed or preceded by the dehydrogenation step q, to deliver the compounds of the invention of general formula XXVI and XXVII, respectively, may be carried out where it appears to be most appropriate.

Further derivatisation of R⁷¹ might be necessary to obtain the desired compounds of general formula XXVI or XXVII, wherein R⁴ represents optionally substituted aryl or heteroaryl as defined above, preferably an aryl or heteroaryl group substituted with one or two substituents independently selected from the group consisting of: —CHO, —CH₂—O—CO—R¹¹, —CH₂—O—CO—NHR¹², —CO—O—R⁹, —CO—NR¹²R¹³, —O—CO—R¹¹, —O—CO—NHR¹², —NR¹⁰—CO—R¹¹, —NR¹⁰O—CO—NHR¹², —NR¹⁰O—CO—O—R¹⁴, —CH₂—NH—CO—NHR¹², —CH₂—NH—CO—R¹¹, and —CH₂—NH—CO—O—R¹⁴, whereby one substituent may also be selected from the group consisting of —CH—O—R⁹, -halogen, —O—R⁹, —NR¹²R¹³, —(C₁-C₄)alkyl, and halogenated —(C₁-C₄)alkyl, wherein R⁹, R¹⁰, R¹¹, R¹², R¹³ and R¹⁴ have the aforementioned meanings. In case that R⁹, R¹⁰, R¹¹, R¹², R¹³ and/or R¹⁴ represent hydrogen, intermediate protection of the oxygen or nitrogen atom might be necessary in the overall reaction scheme.

A) Derivatisation of a —CH₂—OH Substituent in the R⁷¹ Aryl or Heteroaryl Group

If R⁷¹ represents an aryl or heteroaryl group substituted with at least one —CH₂—OH group, the derivatisation may include the oxidation of the —CH₂—OH group into a carbonyl —CHO group, e.g. using a Jones reagent. Alternatively, the oxidation reaction may for example be performed using dimethyl sulfoxide (=DMSO) as oxidizing agent in the presence of an electrophile, for example Dicyclohexylcarbodiimide or oxalyl chloride (so-called “Swern oxidation”). Furthermore, selective oxidation can also be performed with pyridinium chlorochromate (=PCC) as oxidizing agent.

Another option is to perform the oxidation reaction in the presence of a catalytic amount of a stable organic nitroxyl radical. The above reaction may be carried out by electro-oxidation in the presence of the organic nitroxyl radical. Alternatively, the oxidation reaction may be carried out in the presence of a nitroxyl radical and at least one molar equivalent of a co-oxidant selected from the group consisting of m-chloroperbenzoic acid, high-valent metal salts, sodium bromite, sodium or calcium hypochlorite, N-chlorosuccinimide or hypervalent iodine compounds such as [bis(acetoxy)iodo]benzene. Preferably, the co-oxidant is sodium hypochlorite. The stable organic radical preferably comprises a completely α-substituted piperidin-1-oxy radical, such as 2,2,6,6-tetramethyl-1-piperidinyloxy, free radical (TEMPO, free radical). The resulting carbonyl function may be further functionalized (see below).

Another possible modification of the —CH₂—OH substituent comprises the reaction of the free hydroxyl group with a) an appropriately substituted isocyanate R¹²—N═C═O to produce the corresponding compound with the desired R⁴ side chain representing an aryl or heteroaryl group carrying at least one substituent —CH₂—O—CO—NHR¹², or with b) an appropriately substituted carboxylic acid R¹¹—CO—OH or a more reactive derivative thereof (e.g. an acid anhydride or an acid chloride) in an esterification reaction to produce the corresponding compound with the desired R⁴ side chain representing an aryl or heteroaryl group carrying at least one substituent —CH₂—O—CO—R¹¹.

B) Derivatisation of a —COOH Substituent in the R71 Aryl or Heteroaryl Group

If R⁷¹ represents an aryl or heteroaryl group substituted with at least one —COOH group, a reaction may be carried out comprising the modification of the —COOH substituent into an ester or amide derivative by nucleophilic substitution with the appropriate alcohol R⁹—OH or the appropriate amine R¹²R¹³NH by reactions well known to the skilled artisan (e.g. EDCI coupling), thereby resulting in a derivative of general compounds XXIII, XXIV or XXV with a residue R⁴ representing an aryl or heteroaryl group carrying at least one substituent —CO—O—R⁹ and —CO—NR¹²R¹³, respectively.

C) Derivatisation of a —OH Substituent in the R⁷¹ Aryl or Heteroaryl Group

If R⁷¹ represents an aryl or heteroaryl group substituted with at least one —OH substituent, the free hydroxyl substituent may be reacted with a) an appropriately substituted isocyanate R¹²—N═C═O to produce the corresponding compound with the desired R⁴ side chain representing an aryl or heteroaryl group carrying at least one substituent —O—CO—NHR¹², or with b) an appropriately substituted carboxylic acid R¹¹—CO—OH or a more reactive derivative thereof (e.g. an acid anhydride or an acid chloride) in an esterification reaction, to produce the corresponding compound with the desired R⁴ side chain representing an aryl or heteroaryl group carrying at least one substituent —O—CO—R¹¹.

D) Derivatisation of a —NHR¹⁰ or —NH₂ Substituent in the R⁷¹ Aryl or Heteroaryl Group

If R⁷¹ represents an aryl or heteroaryl group substituted with at least one —NHR¹⁰ and/or —NH₂ group, a subsequent reaction may give rise to compounds with at least one —NH—CO—R¹¹, —NH—CO—NHR¹², or —NH—CO—O—R¹⁴, and —NR¹⁰—O—CO—R¹¹, —NR¹⁰—O—CO—NHR¹², or —NR¹⁰—CO—O—R¹⁴ substituent in the aryl or heteroaryl group of R⁴ by reaction of the amine function —NH₂ or —NHR¹⁰ with an appropriately substituted acid halide R¹¹—CO-Hal, an appropriately substituted isocyanate R¹²—N═C═O, and an appropriately substituted chloroformic acid ester R¹⁴—O—CO—Cl, respectively.

E) Derivatisation of a —CH₂—NH₂ Substituent in the R71 Aryl or Heteroaryl Group

If R⁷¹ represents an aryl or heteroaryl group substituted with at least one —CH₂—NH₂ group, a subsequent reaction may give rise to compounds with at least one —CH₂—NH—CO—NHR¹², —CH₂—NH—CO—R¹¹, and —CH₂—NH—CO—O—R¹⁴ substituent in the aryl or heteroaryl group of R⁴ by reaction of the amine function —CH₂—NH₂ with an appropriately substituted isocyanate R¹²—N═C═O, an appropriately substituted acid halide R¹¹—CO-Hal or ester R¹¹—CO—OH, and an appropriately substituted chloroformic acid ester R¹⁴—O—CO—Cl, respectively.

Some of the above described reactions summarized under A, B, C, D or E may preferably be carried out before deketalization of the 3,20-diketo groups.

When an already deketalized 18-(2-R⁷¹-substituted-ethyl)-(9β,10α)-pregna-4-ene-3,20-dione derivative carries a residue R⁷¹ representing an aryl or heteroaryl group substituted with at least one —CH₂—OH group, the oxidation of said —CH₂—OH group to a carbonyl —CHO group as explained above produces a valuable starting compound of general formula XXX for further functionalization

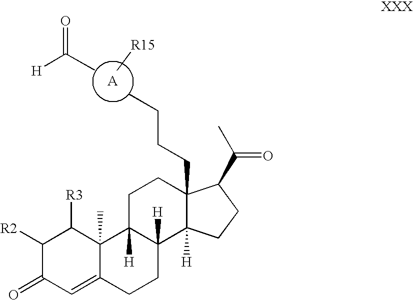

wherein R² and R³ have the meanings as given above;

wherein the ring A represents an aryl or heteroaryl group, and

wherein R¹⁵ represents a substituent selected from the group consisting of hydrogen, —CH₂—OR⁹′; —CO—O—R⁹′, —CO—NR¹²′R¹³′, -halogen, —OR⁹′, —NHR¹⁰′, —NR¹²′R¹³′, —(C₁-C₄)alkyl, halogenated —(C₁-C₄)alkyl, —CH₂—O—CO—R¹¹, —CH₂—O—CO—NHR¹²′, —O—CO—R¹¹′, —O—CO—NHR¹²′, —NR¹⁰′—CO—R¹¹′, —NR¹⁰′—CO—NHR¹²′, and —NR¹⁰′—CO—O—R¹⁴′, and R¹⁵ preferably represents hydrogen; and

wherein R⁹′, R¹⁰′, R¹¹′, R¹²′, R¹³′ and R¹⁴′ have the meanings as given here within for R⁹, R¹⁰, R¹¹, R¹², R¹³ and R¹⁴, but does not represent hydrogen, or represent a conventional protective group PG**.

The derivatisation of the carbonyl function on the aryl or heteroaryl group A may give rise to a substituent selected from the group consisting of: —CH═N—O—R¹⁴, —CH═N—O—CO—NHR¹², —CH═N—O—CO—R¹¹, and —CH═N—O—CO—O—R¹⁴ and may be performed by reactions of the carbonyl function according to the procedures described within U.S. Pat. No. 5,693,628: For example, the carbonyl group may be reacted with a compound of general formula NH₂—O—Y, wherein Y is a hydrogen atom, an —(C₁-C₄)alkyl residue, or a halogenated —(C₁-C₄)alkyl residue, producing a compound with a —CH═N—O—R¹⁴ substituent in A, respectively. The compound of general formula NH₂—O—Y is present in the form of such compound, or in a form from which the compound of the general formula NH₂—O—Y is released under the selected conditions of the reaction. Preferably, the reaction is carried out with equimolar ratios of the corresponding educts. The resulting compound with a —CH═N—OH substituent in the aryl or heteroaryl group A may be modified further by well known reactions of the hydroxyl-imino-methyl group, e.g. formation of the corresponding urethane derivative —CH═N—O—CO—NHR¹² by reaction with an appropriately substituted isocyanate R¹²—N═C═O in inert solvents; esterification to produce the corresponding —CH═N—O—CO—R¹¹ side chain by using acylating agents such as appropriately substituted acid halogenides R¹¹—CO-Hal or acid anhydrides (R¹¹—CO)₂O in the presence of bases; or formation of the corresponding —CH═N—O—CO—O—R¹⁴ derivative by reaction with an appropriately substituted chloro-formic acid ester derivative R¹⁴—O—CO—Cl.

With the above described reactions it is possible to generate the compounds of the invention of general formula XXVI. The optional dehydrogenation step q delivering compounds of the invention of general formula XXVII may be carried out where it appears to be most appropriate in the overall reaction scheme, most preferably before addition of the NH₂—O—Y to the carbonyl function.

An overview of the synthesis of the preferred compounds of the present invention of general formula III or V 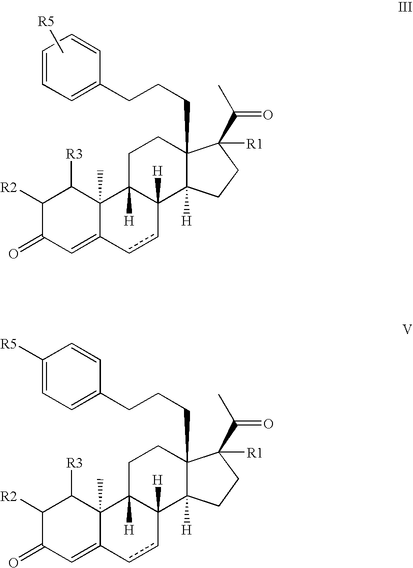
and in particular of compounds of general formula IV or VI

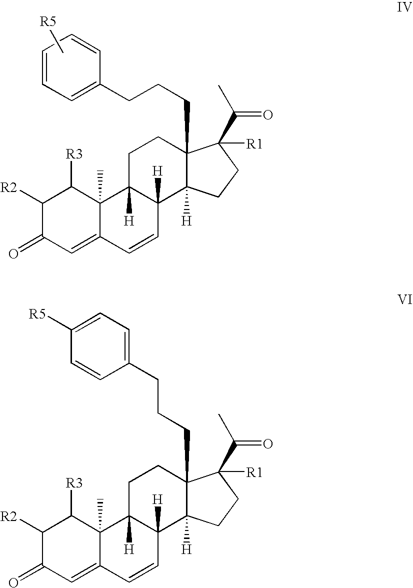

wherein R¹ still represents H (and below designated as compounds of general formula IV-H and VI-H, respectively), is displayed within the following reaction SCHEME VI for compounds of general formula IV. However, it is clear that the same reaction can be applied to deliver compounds of general formula VI:

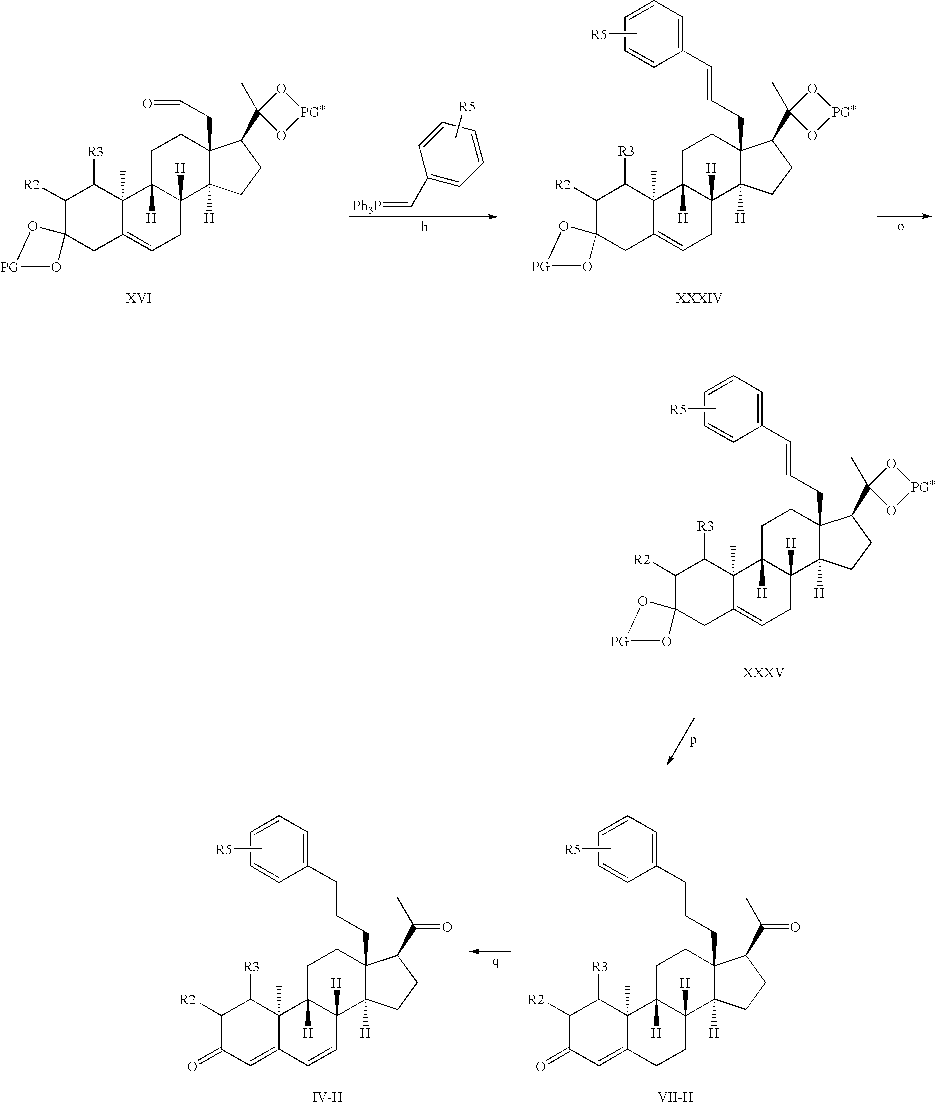

wherein R⁵ smay have the meanings as given here within or a residue from which the desired R⁵ residue can be derived by the above described reactions for derivatisation of the optional substituents of the aryl or heteroaryl groups of R⁷ and/or R⁷¹ to deliver the desired residue R⁴. The reaction step h refers to the Wittig addition, step o to the hydrogenation, step p to the deketalization of the 3, 20 diketo function and step q to the dehydrogenation of the 6,7 bond of the steroid core, as described above. The reactions for derivatisation of R⁵ may be performed when it appears to be most appropriate, i.e. before or after the deketalization (step p) and/or dehydrogenation (step q).
Introduction of a Further Functionality in C17 Position of the Steroidal Core to Generate Compounds of the Invention of General Formula II or VIII

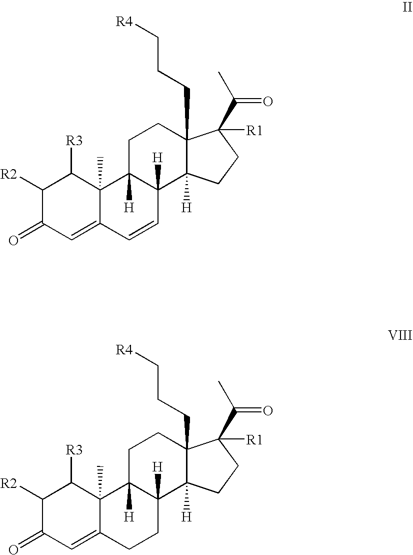

wherein R¹ represents —OH, —O—(C₁-C₄)alkyl, —O—CO—(C₁-C₄)alkyl, and —O—CO—O—(C₁-C₄)alkyl., and wherein R², R³ and R⁴ have the meanings as set out here within.

The derivatisation of the C17 position may be started from different intermediates depending on the stability and reactivity of the R⁴ side chain and its predecessors R⁷ and R⁷¹, and the substituents R¹⁵ or R⁵ in the side chain. Therefore, the starting material is preferably one of the intermediate compounds of general formula XXIV or XXVI or any intermediate there in between, or the corresponding derivatives with the still protected keto functions in C3 and C20 position of general formula XXIII and XXVIII, respectively. However, before further modifying the C17 position, the oxo groups in C3 and C20 position have to be deprotected by deketalization (step p). Accordingly, one of the following intermediate compounds is preferably used as starting material for the derivatisation of the C17 function, whereby any reactive groups in the R⁷, R⁷¹ or R⁴ side chains, such as hydroxyl groups or amino functions have to be protected by adding a suitable protective group PG**.

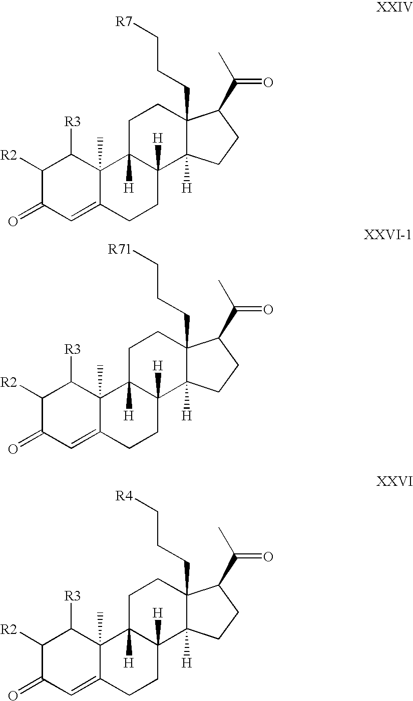

The functionalization of the C17 position starts with the introduction of a —OH group in C17 alpha position as displayed in the following general reaction SCHEME VII (and according to reactions as displayed in U.S. Pat. No. 3,555,053 and by Halkes & van Moorselaar [1969]:

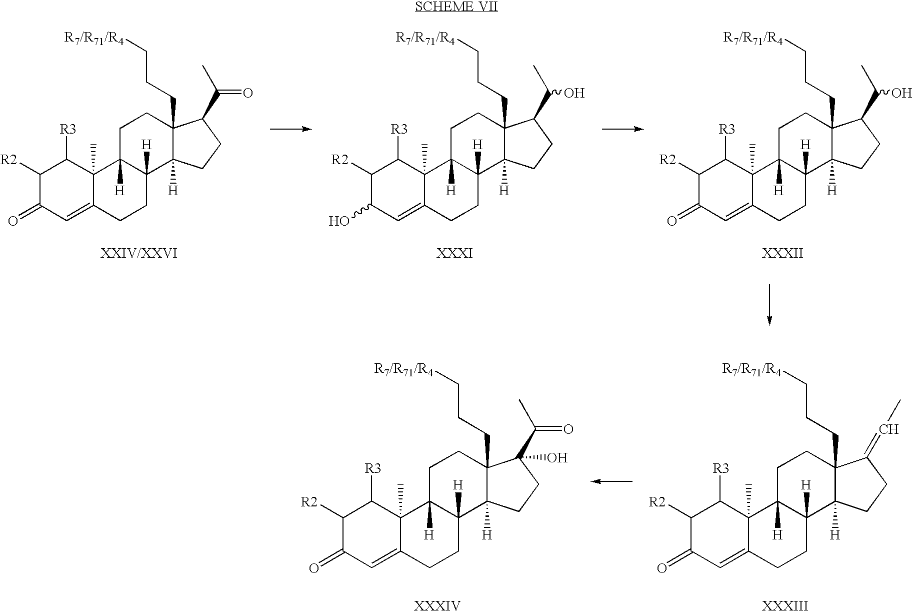

wherein R² and R³, R⁷, R⁷¹ and R⁴ have the meanings as given above, and any reactive groups in the R⁷, R⁷¹ or R⁴ side chains, such as hydroxyl groups or amino functions are protected by a suitable protective group PG**.

The reaction SCHEME VII starts with the reduction of the 18-(R^{7/71/4}-substituted)-ethyl-(9β,10α)-pregna-4-ene-3,20-dione of general formula XXIV or XXVI by using a suitable reducing agent such as lithium aluminium hydride (LAH) to produce the corresponding 3,20-diol of general formula XXXI. The 3-hydroxy group of this general compound is then selectively re-oxidized by means of a selective oxidizing agent such as 2,3-dichloro-5,6-dicyano-p-benzoquinone (DDQ) in an aromatic solvent or manganese dioxide. The resulting 18-(R^{7/71/4}-substituted)-ethyl-20-hydroxy-(9β,10α)-pregna-4-ene-3-one of general formula XXXII was further dehydrated by tosylation with tosyl chloride in pyridine and subsequent treatment of the generated tosylate with boiling pyridine affording the 17,20 unsaturated derivative of general formula XXXIII in a mixture of cis and trans isomers. The latter compound is then oxygenated using an amine oxide such as N-methylmorpholine-N-oxide (NMMO) as stochiometric oxidizing agent and additional hydrogen peroxide in the presence of a catalytic amount of osmium tetroxide to afford the corresponding 17α-hydroxy-18-(R^{7/71/4}-substituted)-ethyl-(9β,10α)-pregna-4-ene-3,20-dione of general formula XXXIV.

The compound of general formula XXXIV may be further modified by subjection to an etherification, esterification or carboxylation reaction at the hydroxyl group at the carbon atom C17 to produce a compound of the general formula XXXV, XXXVI, or XXXVII, whereby the reactions are generally described within Belgian patent specification BE 577,615 or U.S. Pat. No. 3,937,700, and displayed in the following general SCHEME VIII:

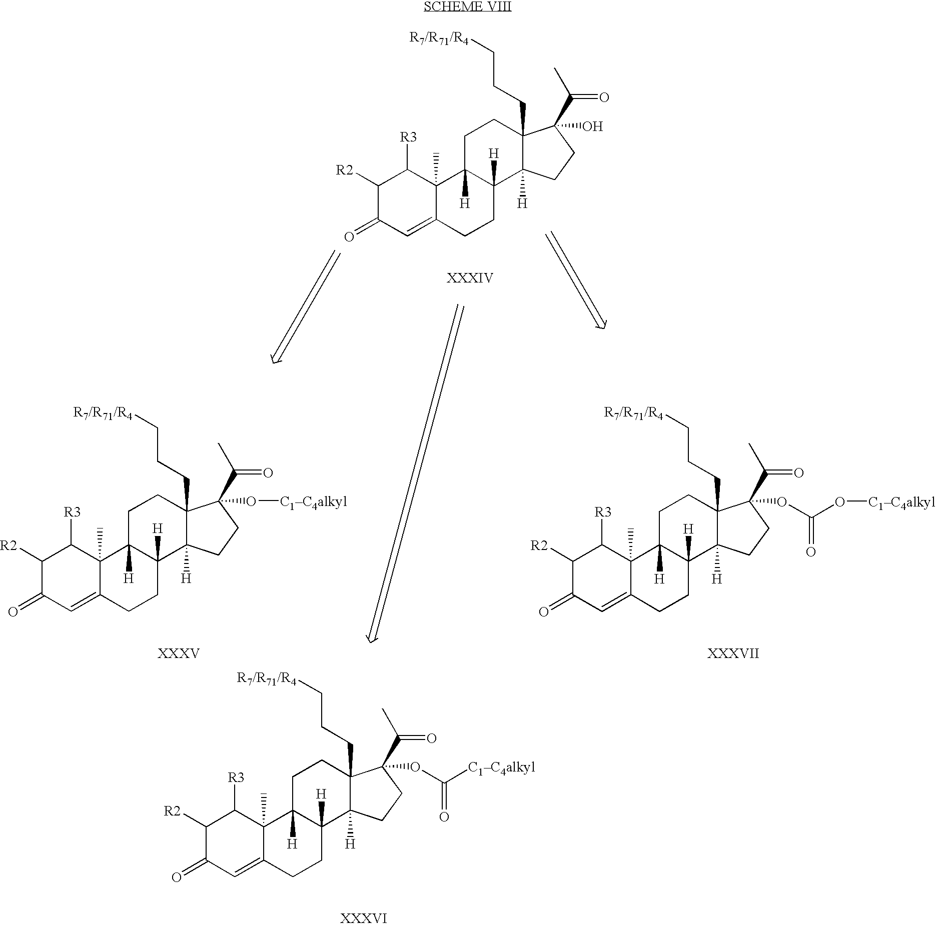

wherein R² and R³, R⁷, R⁷¹ and R⁴ have the meanings as given above, and any reactive groups in the R⁷, R⁷¹ or R⁴ side chains, such as hydroxyl groups or amino functions, are protected by a suitable protective group PG**.

Suitable acylating agents are carboxylic acids, carboxylic acid anhydrides or carboxylic acid chlorides in the presence of a catalyst such as p-toluene sulfonic acid, trifluoroacetic acid, anhydride or pyridine-HCl or in the presence of an acid binder such as an organic base, for example, collidine. The acylation reaction is carried out in the presence of a solvent such as a hydrocarbon, for example, benzene or toluene. The reaction temperature may vary between room temperature and the boiling point of the solvent used. Since—if the starting material contains, apart from the 17-OH group, one or more further OH-groups—these will also be esterified, the further OH-groups have to be protected in advance.

The alkylation reaction may be carried out by the following methods:
1. A reaction with an alkylhalide in the presence of Ag₂O.
2. A reaction of dihydropyrane or dihydrofurane in a weak acidic, weak alkaline or neutral medium.

Carboxylation of the C17 alpha hydroxyl group might be achieved by reaction with an alkylhalide in the presence of Ag₂CO₃.

In order to arrive at the intended compounds of the invention of general formula II or VII, the compounds of general formulas XXXIV, XXXV, XXXVI, or XXXVII are optionally further modified in the R⁷, R⁷¹ and R⁴ residue, respectively, to generate the desired side chain; in particular any protective groups PG** may be removed and the substituents in the aryl or heteroaryl group of R⁷ or R⁷¹, such as a —CH₂—OH, —CO—OH, —OH, —NHR¹⁰, or—CH₂—NH₂ group are further derivatized as explained above. In addition, the dehydrogenation step q to afford the 4,6 unsaturated derivative of general formula II has to be performed where it appears to be most appropriate in the overall reaction scheme.

### BRIEF DESCRIPTION OF THE DRAWINGS

The invention will be described in further detail hereinafter with reference to the accompanying drawing figures in which:

FIG. 1 is a graph showing the antiluteolytic activity of dydrogesterone (a PR agonist), mifepristone (a PR antagonist), and compounds of the invention in guinea pigs assessed by determination of serum progesterone profiles throughout the treatment period from day 10 to day 17 after ovulation.

FIG. 2 is a graph showing the immunohistological score for uterine PR expression in guinea pigs after treatment with dydrogesterone (a PR agonist), mifepristone (a PR antagonist), and compounds of the invention (one bar represents one animal).

### EXPERIMENTAL SECTION

Examples of preparations of compounds of the invention are provided in the following detailed synthetic procedures. In single compound synthesis all reactions were stirred magnetically or shaken with an orbital shaker unless otherwise indicated. Sensitive liquids and solutions were transferred via syringe or cannula, and introduced into reaction vessels through rubber septa, in these cases the reaction were carried out under a positive pressure of dry argon or dry nitrogen. Commercial grade reagents and solvents were used without further purification.

Unless otherwise stated, the term “concentration under reduced pressure” refers to use of a Buchi or Heidolph rotary evaporator (“Rotavapor”) or vaccum centrifuges (“GeneVac”) at approximately 15 mm of Hg. All temperatures are reported uncorrected in degrees Celsius (° C). Unless otherwise indicated, all parts and percentages are by volume.

Thin-layer chromatography (TLC) was performed on Merck™ pre-coated glass-backed silica gel or aluminium sheets 60A F-254 250 μm plates unless stated otherwise. Visualization of plates was effected by one or more of the following techniques: (a) ultraviolet illumination (254 nm or 266 nm), (b) exposure to iodine vapor or iodine vapor and phosphomolybdic acid and subsequent heating, (c) spraying of the plate with Schlittler's reagent solution followed by heating, (d) spraying of the plate with anisaldehyde solution followed by heating, and/or (e) spraying of the plate with Rauxz reagent solution followed by heating.

Melting points (mp) were determined using a Reichert Thermovar melting point apparatus or a Mettler DSC822 automated melting point apparatus and are uncorrected. Proton (1H) nuclear magnetic resonance (NMR) spectra were measured with a Bruker ARX (400 MHz) or Bruker ADVANCE (500 MHz) spectrometer with either Me₄Si (δ 0.00) or residual protonated solvent (CHCl₃δ 7.26; CHD₂ODδ 3.30; DMSO—d₅δ 2.50) as standard. Carbon (¹³C) NMR spectra were measured with a Bruker ARX (100 MHz) spectrometer with either Me₄Si (δ 0.00) or solvent (CDCl₃δ 77.05; CD₃OD δ 49.0; DMSO—d₆δ 39.45) as standard. NMR spectra and elemental analyses of the compounds were consistent with the assigned structures.

### Important Intermediates or Reference Examples

### Detailed Synthesis

### Intermediate 18-formyl-(9β,10α)-pregna-5-ene-3,20-diethylenedioxyketal of formula XVI-H

The synthesis of the first key intermediate 18-formyl-(9β,10α)-pregna-5-ene-3,20-diketal of formula XVI-H

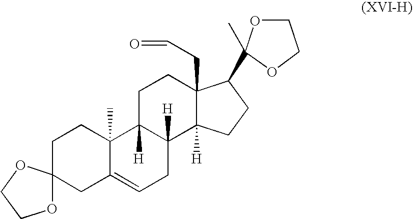

was performed according to the reactions displayed in general SCHEME I, and described in more detail below.

### 9β,10α-Pregna-4-ene-3,20-dione(9β,10α-progesterone or retroprogesterone) [X-H]

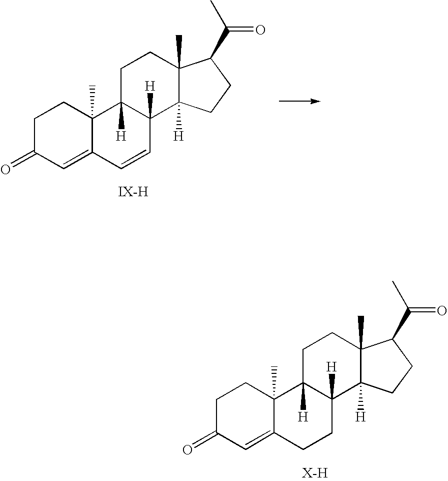

Commercially available Dydrogesterone (9β,10α-pregna-4,6-diene-3,20-dione) of formula IX-H is converted to the corresponding 9β,10α-pregna-4-ene-3,20-dione(9β,10α-progesterone) of formula X-H under reducing conditions (step a).

50 g of Dydrogesterone (160 mmol) were dissolved in 550 ml of toluene. A suspension of 0.75 g of Pd/CaCO₃ (5% Pd) (1.5% of educt) in 100 ml toluene was introduced into a hydrogenation flask, which was flushed with hydrogen. While the mixture was vigorously stirred, the catalyst was hydrogenated. Then the Dydrogesterone solution was injected into the flask; residues of Dydrogesterone were added by rinsing through with 2×50 ml portions of toluene. The hydrogenation was carried out under vigorous stirring until 3.6 l of H₂ have been absorbed (approx. 1 h). Then, the flask was evacuated and flushed with argon (3×). The suspension was suction filtered through diatomaceous earth and rewashed with some toluene. The solvent was removed under a vacuum, and the resulting residue redissolved in approx. 90 ml of DCM. Crystallisation was initiated by addition of 900 ml of warm hexane and completed by leaving the mixture to stand overnight. The crystals formed were removed by suction filtration and rewashed with 100 ml of 10% DCM/hexane. Vacuum-drying gave rise to 36.9 g of (X-H) ([a]D₂₀=−60 (c=1, CHCl₃)). The solvent was completely removed from the mother liquor and the residue (approx. 13 g) was dissolved in approximately 20 ml of DCM. Crystallization was initiated by addition of 150 ml of hexane. After suction filtration and washing, 7.3 g of secondary crystals of (X-H) were obtained. Overall yield: 44.2 g of (X-H) (88%).

### 20-Cyano-20-hydroxy-9β,10α-pregna-4-ene-3,20-dione [XI-H](9β,10α-progesterone 20 cyanohydrine)

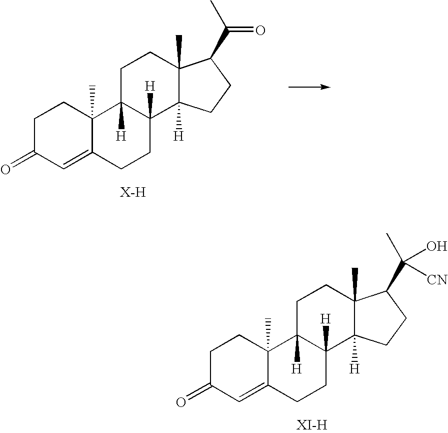

In step b, the 9β,10α-pregna-4-ene-3,20-dione (X-H) is reacted with HCN to produce the corresponding 20-cyano-20-hydroxy compound of formula XI-H.

Apparatus: A 500 ml three-necked flask with mechanical stirrer, internal thermometer, two-neck attachment, dropping funnel and gas discharge tap is prepared with 5 downstream connected wash bottles (1× empty, 1× filled with CaCl₂, 1× empty, 1× conc. KOH and finally alkaline H₂O₂ solution; for absorbing and breaking down excess HCN). On top of the dropping funnel, which has a shut-off tap in the pressure equalisation tube, there is fitted a straight adaptor with a jacketed coil condenser on top. Three additional wash bottles (1× empty, 1× conc. KOH and 1× alkaline H₂O₂ solution), which can be shut off by a tap, are connected to the suction port of the adaptor. A tube for feeding HCN gas is connected via a ground glass connector at the upper end of the jacketed coil condenser. The three-necked flask is fastened in the cooling bath of a circulating condenser; the circulating pump passes cooling liquid through the jacketed coil condenser. The hydrogen cyanide gas is evolved in a 1 l three-necked flask. The flask is placed in a water bath, which is heatable by a magnetic stirrer. Fitted thereon are a dropping funnel (with a gas inlet for argon) and a downwardly inclined (uncooled) distillation bridge. The receiver consists of a 250 ml round-bottomed flask with adaptor, the suction port of which is connected to three further wash bottles connected in series and filled with glass wool and calcium chloride for drying the HCN gas. The receiver and the wash bottles are kept at a temperature of approximately 50° C. in a water bath in order to prevent condensation of the HCN. A tube leads from the final wash bottle to the top of the above described jacketed coil condenser in which hydrogen cyanide is subsequently condensed. All joints are clamped/wired and so secured against being unintentionally loosened. The two outlets from the apparatus (downstream from the series of five wash bottles and downstream from the series of three wash bottles) are passed directly into the fume hood; a gas mask is kept at hand.

Reaction: 1.) Initially, 35 g (111 mmol) of 9β,10α-progesterone were introduced into the 500 ml three-necked flask and suspend in 425 ml of MeOH. The mixture was stirred at RT for 30 min, thereby providing an inert argon atmosphere in the entire apparatus, and then cooled down to approximately −5° C. (under a gentle stream of argon). 4.7 ml of triethylamine (33 mmol) were added to the suspension. 2.) 50 ml of water were introduced into the 1 l three-necked flask, 104 g of conc. H₂SO₄ (95%; 1.0 mol) and 0.6 g of iron(II)sulfate were added, and the water bath was adjusted to 70° C. Then the dropping funnel was charged with a solution of 82 g of sodium cyanide (1.67 mol) in 140 ml of water. 3.) The coolant circulating pump was switched on and the jacketed coil condenser cooled down for condensation of the HCN gas. The HCN evolving by slow dropwise addition of the cyanide solution to the 1 l three-necked flask condensed after some time in the jacketed coil condenser and dripped into the dropping funnel above the 500 ml flask (after approximately 45 min, 58 ml liquid HCN were obtained). Argon was cautiously injected to transport any residual HCN in the apparatus to the jacketed coil condenser. 4.) Then, the liquid HCN was added dropwise over 15 min to the stirred 9β,10α-progesterone suspension. Once the temperature has equalised, the reaction mixture was stirred at −8 to −3° C. for 2 d. The progress of the reaction was controlled by TLC analysis (CHCl3/MeOH 95:5 (vol.:vol.); educt R_{f}: approx. 0.7; product R_{f}: approx. 0.5).

Work up: The apparatus was flushed with argon in order to drive HCN residues into the wash bottles. The dropping funnel above the 500 ml flask was filled with 100 ml of diluted sulfuric acid, which was added dropwise to the reaction mixture for 2 h, whereby excess HCN was driven in the gentle stream of argon into the wash bottles. The reaction mixture was then added to 1 l of precooled (5° C.) DCM, and the final gas residues were removed by bubbling with argon. The phases were separated and the aq. phase is extracted with cold DCM (2×200 ml). The combined organic phases were washed (acidified ice-water, 2×200 ml). After drying (Na₂SO₄) and removal of the solvent under vacuum, 38.1 g of (XI-H) in the form of yellowish crystals were obtained (100% yield).

### 18-Cyano-9β,10α-pregna-4-ene-3,20-dione(18-Cyano-retroprogesterone) [XII-H]

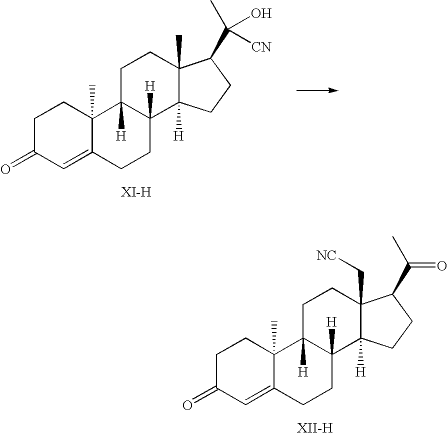

In step c, the 20-cyano-20-hydroxy-9β,10α-pregna-4-ene-3,20-dione of formula XI-H is converted by irradiation in the presence of iodine and lead-tetra-acetate (LTA) to yield the 18-cyano derivative of the formula XII-H.

As apparatus, a 1 l quartz flask with magnetic stirrer, jacketed coil reflux condenser, and argon connection was prepared and equipped with a high pressure mercury vapor lamp (400 W, Philips HPA 400/30 SD-C) and an aluminium reflector (distance approx. 5 cm). The flask was filled with argon, and 24 g of LTA (predried with KOH/argon) were introduced. After addition of 10 g of CaCO₃ and 550 ml of cyclohexane, the suspension was heated to reflux under argon for ½ h, and subsequently cooled to approx. 35° C. A solution of 8.5 g of the cyanohydrin (XI-H) in 250 ml of DCM was prepared and degased with argon, and then added to the lead tetra acetate suspension through the condenser. 2.1 g of iodine were added under a gentle stream of argon. IIIumination was performed in three passes, each of approx. 6 min; in each case the flask was afterwards rotated by a third and further 2.1 g of iodine were added. Then, the encrustations on the internal wall of the flask were removed under a gentle stream of argon, and again 20.1 g of lead tetra acetate and 2.1 g of iodine were added, followed by illumination for a further three passes (each approx. 6 min), performed as described above. The progress of the reaction was controlled by TLC analysis (EtOAc/Hexane 50:50 (vol.:vol.); educt R_{f}: approx. 0.5; product R_{f}: approx. 0.25). Work up: The reaction mixture was allowed to cool and left to settle. The supernatant was kept separately and washed with sodium thiosulfate solution (about 100 g/ml; 2×200 ml). The remaining solid/slurry was rinsed onto a Buchner funnel and suction filtered, and then washed with DCM (2×100 ml). The solid was discarded, and the collected filtrate was shaked with thiosulfate solution. The combined thiosulfate washing solutions were back-extracted with DCM. The organic phases were combined and dried over Na₂SO₄; then the solvent was removed under vacuum. The oily residue is stored at −20° C. After performing the reaction for 4-5 times, the overall obtained reaction products were combined and subjected to column chromatography on silica gel (mobile solvent EtOAc/hexane 50:50). From 38 g of the cyanohydrin XI-H as educt, 24.5 g of (XII-H) were obtained in the form of yellowish frozen foam (65% yield).

¹H-NMR (500 MHz, CDCl₃): 5.75 (m, 1H, H-4, very small couplings); 2.80 (t, 1H); 2.32 (s, 3H, 21-CH3); 1.4 (s, 3H, 19-CH3); 2.55-1.1 (˜21 aliph. H); 1.1 (m, 1H, aliph. H).

### 18-Cyano-9β,10α-pregna-4-ene-3,20-diethylenedioxyketal [XIII-H]

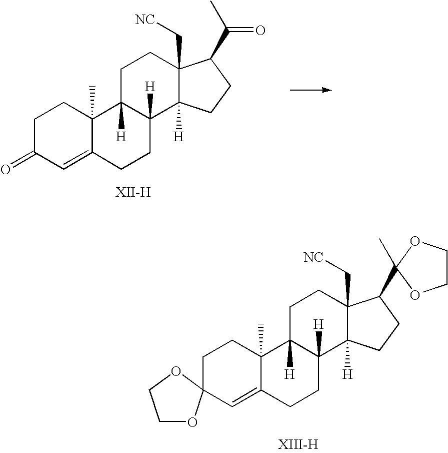

In step d, the two oxo groups of the 18-cyano-9β,10α-pregna-4-ene-3,20-dione of formula XII-H are protected by ketalization with ethyleneglycol to produce the 18-Cyano-9β,10α-pregna-4-ene-3,20-diethylenedioxyketal of formula XIII-H.

21.5 g of the 18-cyano-retroprogesterone (XII-H) (63.3 mmol) were suspended in 150 ml of ethylene glycol. 21 ml of TMOF (0.19 mol) were added and stirred for approximately ¼ h. Then, 150 ml of heptane and 50 ml of dioxane were added. The mixture was stirred at RT for ½-1 h. 0.12 g of pTosOH hydrate were added and the mixture was again stirred overnight at RT. After 18 h the reaction mixture was controlled by TLC analysis (EtOAc/hexane 50:50 (vol:vol); educt R_{f}: approx. 0.2; product R_{f}: approx. 0.5). After addition of 1 ml of collidine to the reaction mixture, 700 ml of toluene and 900 ml of half-saturated NaHCO₃ solution were added and the mixture was stirred thoroughly for about 5 min. The phases were separated and the aq. phase was extracted with toluene (2×200 ml). The combined organic phases were washed (3×400 ml H₂O), whereby the aq. washings were combined and back-extracted with toluene. The combined organic phases were dried over K₂CO₃. The solvent was removed under vacuum pressure. The residue was boiled in 200 ml of DEE and cooled with stirring. After approximately 1 h, the mixture was suction filtered and additionally washed with some DEE to yield 15.5 g of yellowish crystals of (XIII-H).

The mother liquors were evaporated under vacuum, and the residue was suspended in 200 ml of diisopropyl ether and briefly brought to boiling. Complete dissolution was achieved by addition of some isopropanol (5 ml). After addition of approximately 5 g of activated carbon, the mixture was again brought to boiling and slowly cooled with stirring (1 h). The mixture was suction filtered through diatomaceous earth and rewashed with diisopropyl ether. The filtrate was evaporated down to approximately 80 ml, briefly boiled and slowly cooled with stirring (18 h). The secondary crystals are removed by suction filtration, washed with DEE and dried under vacuum to yield further 3 g of yellowish crystals of (XIII-H).

The solvent was removed from the mother liquor under a vacuum, the residue (approx. 8 g) subjected to column chromatography (Al₂O₃ (neutral), mobile solvent: MTBE/hexane approx. 75:25) and further 4.2 g of (XIII-H) were obtained in the form of yellowish frozen foam. The overall yield of (XIII-H) was 22.7 g (84%)

¹H-NMR (500 MHz, CDCl₃): 5.23 (s, 1H, H-4); 4.20-3.85 (m, 8H, ethylene-H); 2.56-2.33 (m, 4H, aliph. H); 2.22-1.52 (m, ˜17H, aliph. H); 1.29 (s, 3H, 21-CH3); 1.20 (s, 3H, 19-CH3); 0.95 (m, 1H, aliph. H).

### 18-Cyano-9β,10α-pregna-5-ene-3,20-diethylenedioxyketal [XV-H]

In steps e and f, the 18-cyano-3,20-diketal derivative of formula XIII-H is transformed into the corresponding Δ5-18-cyano-3,20-ketalized dione of formula XV-H by isomerization, partial deketalization and chromatographic separation of the resulting mixture of the Δ5-diketal and the Δ4-20-monoketal derivatives.

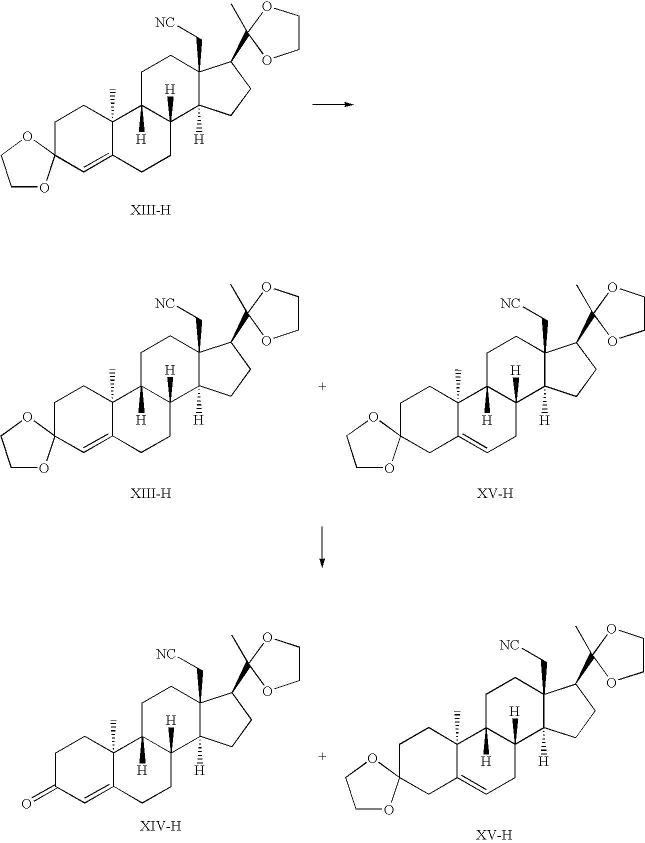

In a 500 ml round-bottomed flask with magnetic stirrer, reflux condenser with 4 Å molecular sieve cartridge/through-flow extractor and argon flask, initially 200 ml of benzene, 53 g of glycol and 0.25 g of p-TosOH hydrate were introduced and heated under reflux, whereby the condensation product dripped through the molecular sieve cartridge (approximately 1-2 h). The solution was cooled down. Then, a solution of 24.1 g of the diketal XIII-H (56.4 mmol) in 100 ml of benzene was added. Under an inert argon atmosphere, the mixture was continuously heated under reflux. The isomerisation process was completed after approximately 8-16 h; the progress of the reaction was controlled by TLC analysis (EtOAc/ hexane 20:80 (vol.:vol.); Δ4 (XIII-H): approx.: 0.2; Δ5 (XV-H): R_{f}: approx. 0.22; monoketal (XIV-H): R_{f}: approx. 0.1). Work-up: 10 g of K₂CO₃ (anhydrous) were added and the mixture was stirred thoroughly (5 min). Then, 0.5 l of half-saturated NaHCO₃ solution was added and the resulting mixture was again thoroughly stirred. The phases were separated and the aq. phase was extracted with benzene (3×150 ml). The combined organic phases were washed (200 ml saturated NaHCO₃ solution, 200 ml water; the washings were back-extracted), dried over K₂CO₃) and the solvent was removed under vacuum.

The resultant isomer mixture (Δ-4/Δ-5) (approximately 25 g) was then dissolved in a 5 l three-necked flask in 900 ml of ACN. 380 ml of borate buffer (5 g of NaB₄O₇×10H₂O dissolved in 500 ml water, brought up to pH 8 by addition of 18% HCl solution) were added. Then, a solution of 977 mg of cerium ammonium nitrate (Ce(NH₄)₂(NO₃)₆, 1.78 mmol) dissolved in 20 ml of water and 20 ml of ACN was added to the isomer mixture and the resulting mixture was stirred at RT for 15 min. Control was performed by TLC analysis. After a reaction time of 20 min, 1.3 l of water and 1.3 l of DEE were added and the mixture was thoroughly stirred. The phases were separated and the aq. phase was extracted with DEE (2×750 ml). The combined organic phases were washed and dried (Na₂SO₄). The residue from which the solvent had been removed was subjected to column chromatography (Al₂O₃ neutral, mobile solvent: MTBE/hexane 75:25). 9.1 g of colorless crystals of XV-H were obtained (38% yield) (melting point: 150-151° C.).

After further elution, approximately 11.1 g of the monoketal (XIV-H) were obtained as frozen foam. The monoketal may be again ketalised, in a similar manner to the reaction step d, to yield the diketal (XIII-H) and be reutilized in this manner.

### 18-Formyl-9β,10α-pregna-5-ene-3,20-diethylenedioxyketal [XVI-H]

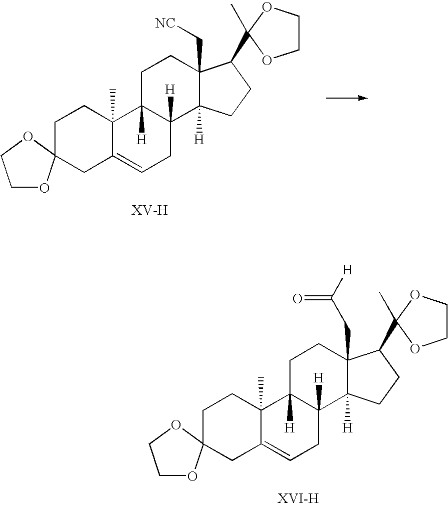

In step g, the Δ5-18-cyano-3,20-diketal of formula XV-H is treated with a reducing agent such as diisobutyl-aluminium-hydride (DIBAH) to gave an aldimine intermediate, which is hydrolyzed to the desired 18-formyl-(9β,10α)-pregna-5-ene-3,20-diketal compound of formula XVI-H.

In a 250 ml round-bottomed flask, which is preheated under argon, with magnetic stirrer, septum, reflux condenser with argon connection and bubble counter, 2.5 g of 18-cyano-Δ-5-diketal (XV-H) (5.85 mmol; dried under a vacuum over P₂O₅) were introduced and dissolved in 110 ml of toluene under provision of an inert argon atmosphere. The solution was cooled to 0-5° C. and then 7.25 ml of DIBAH (20% in toluene, 0.86 g/ml) were added dropwise by syringe. The mixture is stirred for further 15 min. Then 24 ml of ethanol were added, followed by the addition of 60 ml H₂O and 2.5 ml of 2 N NaOH solution. The mixture was heated to reflux and boiled until gas evolution has ceased (about ½ h). The reaction mixture was cooled down and separated into the phases. The aq. phase was diluted with saturated NaCl solution and saturated NaHCO₃ solution (each 50 ml) and extracted with toluene (2×75 ml). The combined organic phases were washed (half-saturated NaHCO₃ solution) and dried (MgSO₄). The solvent was removed under vacuum down to a residue of approximately 15-20 ml (bath temperature 40° C.). This crude solution containing the product (XVI-H) was not further purified but immediately used in the further reaction step h—the addition of the Wittig reagent—by dropwise addition to the ylide solution, which has been prepared in parallel and was available to hand, and reacted without delay to yield the corresponding Wittig adduct of general formula XVII.

Preparation of the Wittig reagent Ph₃P═CH₂—R⁷, wherein R⁷ represents hydrogen or an optionally substituted heteroaryl or aryl residue. The Wittig reagent—the Ph₃P═CH—R⁷ Phorphoran (or Phosphonium-Ylid)—was always freshly prepared from the corresponding Triphenylphosphonium halogenide salt (Ph₃P—CH₂—R⁷)Hal, which is commercially available or can be synthesized by methods known to the skilled artisan, by contact with a strong base such as Phenyllithium or Butyllithium.

The residue R7 represents hydrogen or a heteroaryl or aryl residue, which is optionally substituted in the heteroaryl or aryl group with one or two substituents independently selected from the group consisting of —CH₂—O—PG**; —CH₂—O—R⁹′, —CO—O—PG**, —CO—O—R⁹′, —CO—NR¹²′R¹³′, —CN, -halogen, —O—PG**, —O—R⁹′, —N(PG**)₂, —NPG**R¹⁰, —NR¹²′R¹³′, —(C₁-C₄)alkyl, and halogenated —(C₁-C₄)alkyl, whereby PG** represents a conventional protecting group for the hydroxyl or amine function, and whereby R⁹′, R¹²′ and R¹³′ represent —(C₁-C₄)alkyl or halogenated —(C₁-C₄)alkyl, or R¹²′ and R¹³′ form together with the nitrogen atom, where they are attached, a heterocyclic 4-, 5-, 6-, 7- or 8-membered ring system, which is saturated, partly unsaturated, or aromatic; and which optionally contains 1, 2 or 3 additional heteroatoms selected from N, O or S, the number of additional N atoms being 0, 1, 2 or 3 and the number of O and S atoms each being 0, 1 or 2; and which ring is optionally part of a multiple condensed ring-system. Alternatively, the aryl moiety of R⁷ is optionally substituted by two groups which are attached to adjacent carbon atoms and are combined into a saturated or partly unsaturated cyclic 5, 6, 7, or 8 membered ring system, optionally containing 1, 2 or 3 heteroatoms selected from N, O and S, the number of N atoms being 0, 1, 2 or 3 and the number of O and S atoms each being 0, 1 or 2.

### Preferred Examples for the Wittig Reagent Include

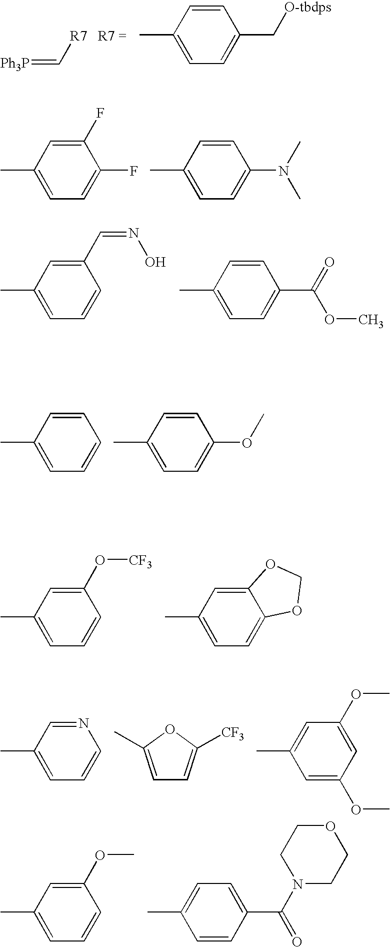

### EXAMPLES

### Detailed Synthesis

In order to more fully illustrate the nature of the invention and the manner of practicing the same, the following examples are presented, but they should not be taken as limiting.

### 1.) 18-(2-Hydroxyethyl)-9β,10α-pregna-4-ene-3,20-dione (No. 40) and 18-(2-Hydroxyethyl)-9β10α-pregna-4,6-diene-3,20-dione(=18-(2-Hydroxyethyl)-dydrogesterone) (No. 41)

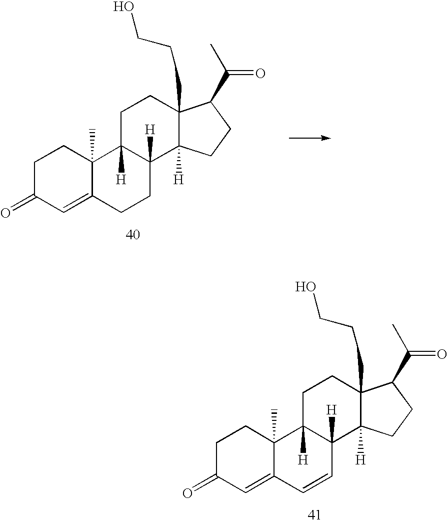

These two compounds of the invention were obtained by reactions as displayed within general reaction SCHEMES II (step h, Wittig addition) and III.

### 1.a): 18-Vinyl-9β,10α-pregna-5-ene-3,20-diethylenedioxyketal (XVII-H)

This intermediate compound was prepared by a Wittig addition according to the following scheme:

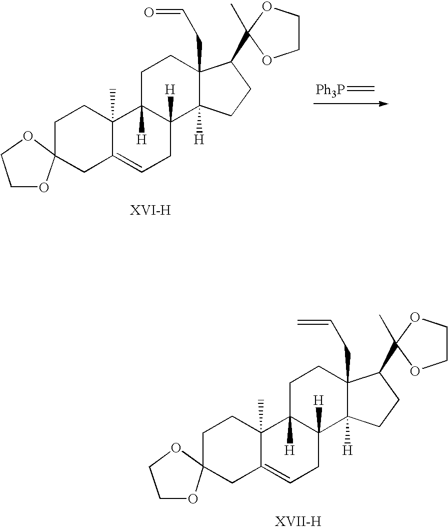

12.5 g of dried methyltriphenylphosphonium bromide (35.1 mmol) was suspended in 200 ml of THF. Under inert argon atmosphere, the mixture was cooled with dry ice/acetone to approximately −65° C. Then, 14 ml of BuLi (2.5 M in hexane; 35.1 mmol) were added dropwise. The mixture was stirred at −65° C. for 30 min; then the temperature was raised to RT over 30 min. The solution was further stirred until it appeared almost clear (˜1 h). Then, the solution was cooled to −65° C., and 1.5 g of the crude 18-formyl-Δ-5-diketal (XVI-H) (dissolved in 20 ml of toluene) (3.51 mmol) was added. The mixture was continuously stirred at −65° C. for ½ h, and then the temperature was slowly raised to RT for ½ h and further stirred at RT overnight. The progress of the reaction was controlled by TLC analysis (EtOAc/hexane 30:70 (vol.:vol.). Educt: R_{f} approx. 0.25; product: R_{f} approx. 0.5). After 24-48 h reaction time, the batch was added to a mixture of 0.5 l water and 200 ml of EtOAc and thoroughly stirred. The phases were separated and the aq. phase was extracted with EtOAc (2×200 ml). The combined organic phases are washed (saturated NaHCO₃), dried (Na₂SO₄) and the solvent was removed under vacuum. The resulting residue was subjected to column chromatography on silica gel (mobile solvent MTBE/hexane approx. 10:90). Yield: 1.0 g of (XVII-H) was obtained as colorless frozen foam.

¹H-NMR (500 MHz, CDCl₃): 6.1 (m, 1H, olefin. H); 5.35 (m, 1H, H-6); 5.05 (d, 1H, olefin. H); 4.95 (d, 1H, olefin. H); 4.05-3.80 (m, 8H, ethylene H); 2.6 (dd, 1H, aliph. H); 2.35-1.1 (m, 21H, aliph. H); 1.33 (s, 3H, 21-CH₃); 1.21 (s, 3H, 19-CH₃).

### 1.b): 18-(2-Hydroxyethyl)-9β,10α-pregna-5-ene-3,20-diethylenedioxyketal (XVIII-OH)

The vinyl derivative of formula XVII-H was transformed into the 18-(2-Hydroxyethyl)-9β,10α-pregna-5-ene-3,20-diethylenedioxyketal by hydroboration and subsequent oxidation according to the following scheme (step i in SCHEME III):

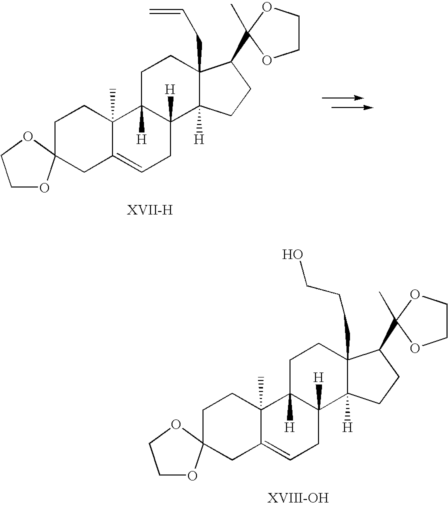

0.79 g of 18-vinyl-9β,10α-pregna-5-ene-3,20-diethylenedioxyketal 3,20-diethylenedioxy-ketal (XVII-H) (1.84 mmol) were dissolved in 10 ml of THF. Under inert argon atmosphere, the solution was cooled with dry ice/acetone to −65° C.; then 2 ml of BH₃-THF (1M, 2.0 mmol) were added and the mixture was stirred and slowly raised to −30° C. (for 1 h), and further stirred at −30 to −20° C. for additional 2-3 h. The progress of the reaction was controlled by TLC analysis (EtOAc/hexane 40:60 (vol.:vol.); educt R_{f} approx. 0.8; product R_{f} approx. 0.45: a sample of the reaction mixture was added to ¼ ml of 2 N NaOH and shaken; after 1 min, 5 drops of H₂O₂ and some MTBE were added and again shaken; the spots were visualized as usual). Work-up: The temperature was raised to approx. −10° C. and 10 ml of 2 N NaOH were added. The mixture was stirred at 0° C. for 15 min, then 2 ml of H₂O₂ solution (35%) were added and the mixture was again stirred at 35° C. for 15 min. This batch was added to a mixture of 50 ml water and 50 ml of DEE and thoroughly shaken. The phases were separated and the aq. phase was extracted with DEE (2×20 ml). The combined organic phases were washed, dried and the solvent was removed under vacuum. Yield: 0.83 g of (XVIII-OH) were obtained (100%) as a colorless frozen foam.

### 1.c): 18-(2-Hydroxyethyl)-9β,10α-pregna-4-ene-3,20-dione (No. 40)

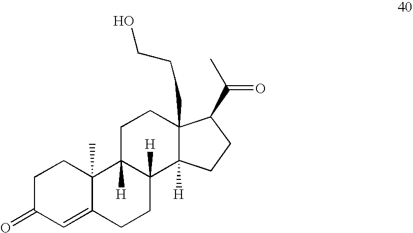

The diketal derivative of formula XVIII-OH was converted into the 18-(2-Hydroxyethyl)-9β,10α-pregna-4-ene-3,20-dione (No. 40) according to step k in general SCHEME III: 0.83 g of the 18-(hydroxyethyl)-9β,10α-pregna-5-ene-3,20-diethylenedioxyketal (XVIII-OH) (1.87 mmol) were dissolved in 40 ml of acetone. Then, 3 ml of 18% H₂SO₄ were added and the mixture was stirred at RT for approximately 18 h. The progress of the reaction was controlled by TLC analysis (EtOAc/hexane 40:60 (vol.:vol.); educt R_{f}: approx. 0.45; product R_{f}: approx. 0.2). Working-up: The batch was diluted with 150 ml of half-saturated NaHCO₃ solution and stirred with DEE (150 ml). The phases were separated and the aq. phase was extracted with DEE (2×100 ml). The combined organic phases were washed (H₂O, saturated NaCl), dried (Na₂SO₄) and the solvent was removed under vacuum. The resulting substance was dissolved in 20 ml of warm MTBE, seeded and left to stand overnight at +5° C. in order to crystallize. The crystals were removed by suction filtration and dried under vacuum. Yield: 317 mg of colorless crystals (No. 40) were obtained (approx. 47%). The crude mother liquor was also further used.

¹H-NMR (500 MHz, CDCl₃): 5.73 (s, 1H, H-4); 3.5 (m, 2H, 2×H-23, becomes triplet after D₂O exchange); 2.6-1.1 (m, 25H, aliph. H and OH); 2.23 (s, 3H, 21-CH₃); 1.39 (s, 3H, 19-CH₃).

### 1.d): 18-(2-Acetoxyethyl)-9β,10α-pregna-4-ene-3,20-dione (XX-Ac)

In order to reintroduce the second double bond in the 6,7-position of the steroidal core, the free hydroxyl group has to be protected first as displayed in the following scheme (corresponding to step I in general SCHEME III):

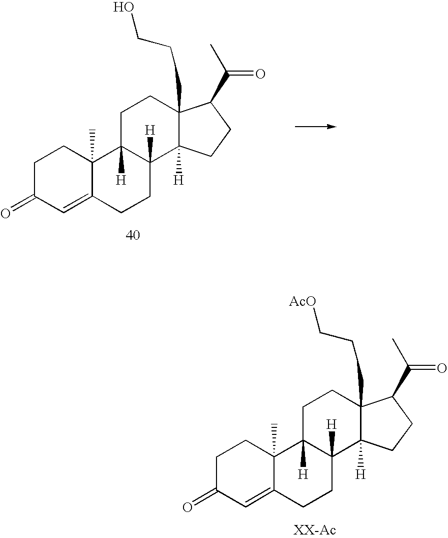

0.42 g of 18-(2-hydroxyethyl)-9β,10α-pregna-4-ene-3,20-dione (No. 40) (crude, about 1.17 mmol) were dissolved in 20 ml of CH₂Cl₂. First 14.3 mg of 4-(N,N-dimethylamino)-pyridine (DMAP) (117 μmol) and then 287 μl of pyridine (3.5 mmol) were added. After addition of 122 μl of Ac₂O (1.29 mmol), the reaction mixture was stirred at RT for 8 h (TLC analysis: EtOAc/hexane 40:60 (vol.:vol.), educt R_{f}: approx. 0.2; product R_{f}: approx. 0.5). Work-up: The solvent was removed under vacuum; the residue was redissolved in MTBE (75 ml). Washing was performed 2× with 20 ml 1% HCl, 30 ml ½ saturated NaHCO₃. The combined organic phases were dried (Na₂SO₄) and the solvent was removed under vacuum. The substance was dissolved in 3-4 ml of warm MTBE, hexane was added until the onset of turbidity, the batch seeded and left to stand overnight at RT in order to crystallize. The crystals were removed by suction filtration and dried under vacuum. Yield: 282 mg of colorless crystals (XX-Ac) were obtained (60%).

¹H-NMR (500 MHz, CDCl₃): 5.67 (s, 1H, H-4); 3.88 (m, 2H, 2×H-23); 2.5-1.1 (m, 24H, aliph. H); 2.15 (s, 3H, 21-CH₃); 1.98 (s, 3H, OAc); 1.32 (s, 3H, 19-CH₃).

### 1.e): 18-(2-Acetoxyethyl)-9β,10α-pregna-4,6-diene-3,20-dione (XXI-Ac)

The 18-(2-Acetoxyethyl)-9β,10α-pregna-4-ene-3,20-dione (XX-Ac) is converted into the corresponding 4,6 unsaturated derivative by dehydrogenation as displayed in the following scheme (corresponding to step m in general SCHEME III):

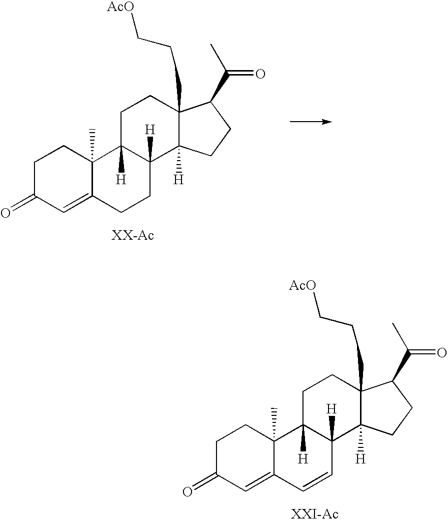

In a 100 ml pointed flask with septum and magnetic stirrer, 25 ml of anhydrous dioxane and—under ice cooling—gaseous hydrogen chloride were introduced; the hydrochloric acid content was determined and diluted with dioxane such that a content of approximately 90 mg of HCl/ml was established. A column (4×18 cm) was packed with a slurry of aluminium oxide (neutral) in DEE and cautiously conditioned with ethereal hydrochloric acid (200 ml, containing approx. 40 mg HCl/ml). The column was rinsed with 200 ml DEE. Then 317 mg of the acetoxy steroid (XX-Ac) (819 μmol) and 240 mg of DDQ (1.06 μmol) were dissolved in 3 ml of dioxane (anhydrous), and stirred at RT until dissolution. This solution was added to the above (titrated) hydrochloric acid/dioxane solution by cannula with stirring. The reaction mixture was further stirred at RT for 20 min. Then the batch was diluted with 150 ml of DEE and immediately introduced into the above-described aluminium oxide column. The column was rinsed with 1 l of DEE and further eluted with EtOAc/hexane 50:50. In total 20 fractions, each of 200 ml, were obtained. Nonpolar secondary products were eluted first. Subsequent fractions (containing product) were combined and the solvent was removed under vacuum. The residue was ultrapurified by chromatography on silica gel (mobile solvent DCM/MeCN approx. 97:3 to 90:10). Yield: 205 mg of (XXI-Ac) were obtained as yellow oil (65%).

### 1.f): 18-(2-Hydroxyethyl)-9β,10α-pregna-4,6-diene-3,20-dione (No. 41)

The 18-(2-Acetoxyethyl)-9β,10α-pregna-4,6-diene-3,20-dione (XXI-Ac) is deprotected to deliver the desired 18-(2-Hydroxyethyl)-9β,10α-pregna-4,6-diene-3,20-dione—compound No. 41 of the present invention—by a reaction corresponding to step n in general SCHEME III.

In a 100 ml pointed flask with magnetic stirrer, 205 mg of the acetoxy steroid (XXI-Ac) (514 μmol) were introduced and dissolved in 11 ml of dioxane. 32.5 mg of LiOH-H₂O (772 μmol) dissolved in 2.5 ml of water were added and the mixture was stirred at RT for 4 h. (TLC analysis was performed with EtOAc/hexane 40:60 (vol.:vol.); educt R_{f} approx. 0.3; product R_{f} approx. 0.1). Work-up: 200 ml of water were added to the reaction mixture and extracted by shaking with 3×50 ml of MTBE. The combined organic phases were washed (H₂O) and dried (Na₂SO₄). After removal of the solvent, chromatographic purification was performed (mobile solvent: EtOAc/hexane 40:60 to 70:30). After careful drying under a high vacuum, 154 mg of compound No. 41 was obtained as yellowish solid (83%).

¹H-NMR (500 MHz, CDCl₃): 6.1 (m, 2H, H-5, H-6); 5.59 (s, 1H, H-4); 3.65 (t, 2H, 2×H-23); 2.99 (dd, 1H, aliph. H); 2.5-0.95 (m, 21H, aliphatic H, OH); 2.05 (s, 3H, 21-CH₃); 1.26 (s, 3H, 19-CH₃). (According to NMR, the substance still contained approximately 10% of an unidentified impurity, which could not be separated.)

### 2.) 18-(2-[4-Hydroxymethylphenyl]-ethyl)-9β,10α-pregna-4-ene-3,20-dione (No. 7)

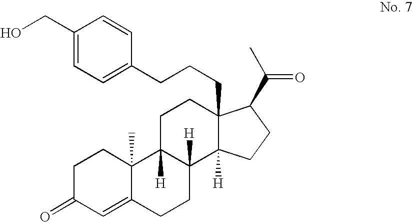

This compound No. 7 of the invention was obtained by reactions as displayed within general reaction SCHEMES II (step h, Wittig addition), IV, V and VI, respectively.

### 2.a) 18-(2-[4-(TBDPS-oxymethyl)-phenyl]-vinyl)-9β,10α-pregna-5-ene-3,20-diethylenedioxyketal (XVII-2)

This intermediate compound was prepared by a Wittig addition according to the following scheme:

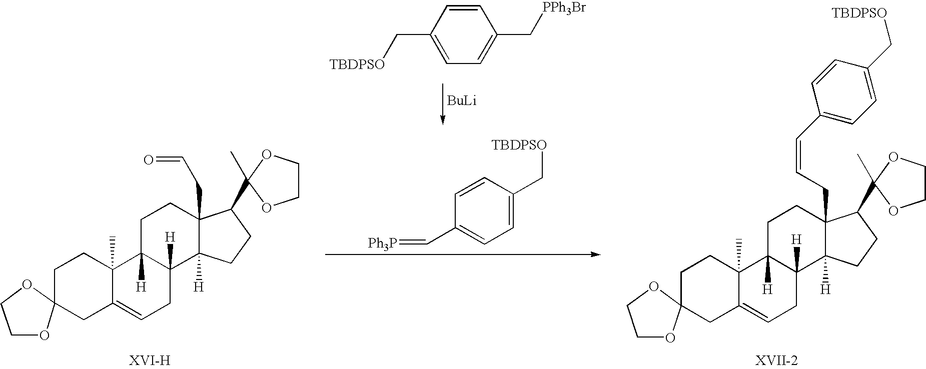

8.2 g of the dried phosphonium salt (11.7 mmol) were introduced and suspended in 250 ml of anhydrous THF. Under inert argon atmosphere, the mixture was cooled with dry ice/acetone to −65° C.; then 5 ml of BuLi (2.5 M in hexane; 12.6 mmol) were added dropwise. Stirring was continued for 30 min at −65° C., then the temperature was raised to RT over 30 min, and the mixture was stirred for further 15 min, until the solution became almost clear. Then, the solution was again cooled down to −65° C. and the toluene solution of the 18-formyldiketal (XVI-H) (2.5 g of crude 18-formyl-Δ-5-diketal (XVI-H), dissolved in 20 ml of toluene) (5.85 mmol) was added dropwise over 5 min. Stirring was continued at −65° C. for further 30 min; then the temperature was slowly raised to RT over 1 h and the mixture was further stirred at RT overnight under a very gentle stream of argon. (TLC analysis with EtOAc/hexane 30:70 (vol.:vol.); educt R_{f} approx. 0.2; product R_{f} approx. 0.5). Work-up: After approx. 24-48 h, the batch was added to a mixture of 1 l water and 250 ml of EtOAc and thoroughly stirred. The phases were separated and the aq. phase was extracted with EtOAc (3×250 ml). The combined organic phases were washed (saturated NaHCO₃, H₂O), dried (Na₂SO₄) and the solvent was removed under vacuum. The residue was subjected to column chromatography on silica gel (mobile solvent MTBE/hexane approx. 20:80). Yield: 2.09 g of (XVII-2) were obtained as colorless frozen foam; the compound was present as a cis/trans mixture and was unambiguously characterized only after further hydrogenation.

### 2.b) 18-(2-[4-(TBDPS-oxymethyl)-phenyl]-ethyl)-9β,10α-pregna-5-ene-3,20-diethylenedioxyketal (XXIII-2)

The next reaction step starting from compound XVII-2 was the reduction of the unsaturated side chain to produce the corresponding 18-(2-[4-(TBDPS-oxymethyl)-phenyl]-ethyl)-(9β,10α)-pregna-5-ene-3,20-diketal XXIII-2 according to the scheme (corresponding to step o in general SCHEME IV):

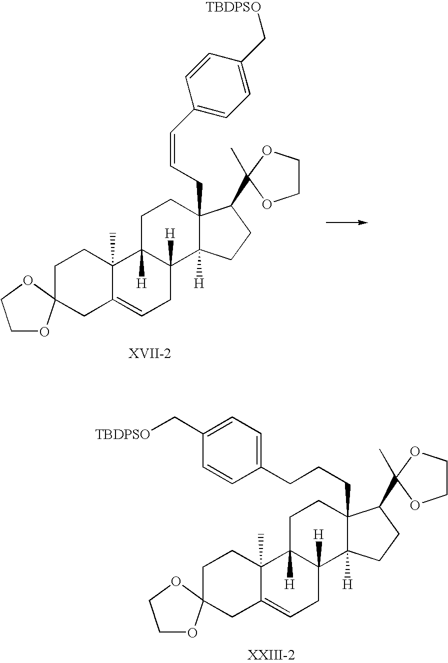

330 mg of catalyst (palladium on calcium carbonate (5%)) were suspended in 30 ml toluene and 36 ml ethanol. Then the catalyst was hydrogenated by stirring under H₂ atmosphere. 2.05 g of the Wittig adduct (XVII-2) (2.65 mmol) were dissolved in 30 ml of toluene, degased with argon and added to the hydrogenated catalyst and hydrogenated under vigorous stirring for 3 h (TLC analysis: EtOAc/hexane 20:80 (vol.:vol.); educt R_{f} approx. 0.7; product R_{f} approx. 0.72). Work-up: The reaction mixture was suction filtered through a bed of diatomaceous earth and rewashed with toluene. The filtrate was evaporated under vacuum. Yield: 2.1 g of (XXIII-2) were obtained as colorless frozen foam.

¹H-NMR (500 MHz, CDCl₃): 7.1-7.7 (14H, arom. H); 5.35 (bs, 1H, olefin. H); 4.75 (d, 2H, benzyl. H); 3.75-4.00 (m, 8H, ethyleneketal-H); 1.1 (s, 9H, t.-Bu); 0.8-2.65 (aliphat. H).

### 2.c) 18-(2-[4-Hydroxymethyl)-phenyl]-ethyl)-9β,10α-pregna-5-ene-3,20-diethylenedioxyketal (XXIII-3)

The next reaction step starting from compound XXIII-2 was the removal of the protection group in the side chain to produce the corresponding 18-(2-[4-Hydroxymethyl)-phenyl]-ethyl) steroid XXIII-3 according to the following scheme:

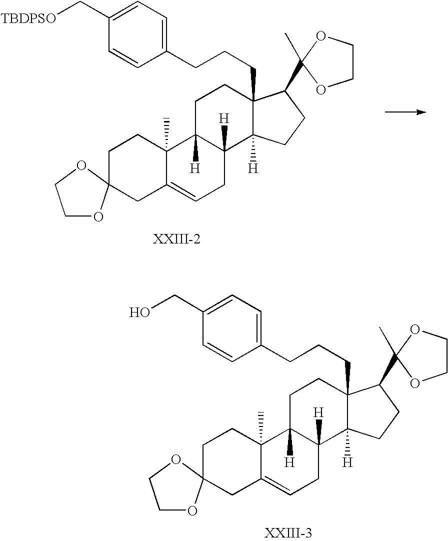

2 g of the steroid (XXIII-2) (2.6 mmol) were dissolved in 40 ml of THF. After addition of 2.71 ml of tetrabutylammonium fluoride (TBAF) (1M in THF, 5% H₂O; 2.71 mmol), the mixture was stirred at RT for 1.5 h (TLC analysis: EtOAc/hexane 40:60 (vol.:vol.); educt R_{f} approx. 0.6; product R_{f} approx. 0.2). Work-up: The solvent was removed under vacuum and the residue redissolved in 50 ml of DEE and 50 ml of water. After thorough stirring, the phases were separated and the aq. phase was extracted with DEE (2×50 ml). The combined organic phases were washed (H₂O) and dried (MgSO₄). After removal of the solvent under vacuum, the residue was chromatographed on silica gel (mobile solvent MTBE/hexane approx. 30:70 to 75:25). Yield: 1.36 g of (XXIII-3) was obtained as colorless frozen foam.

¹H-NMR (500 MHz, CDCl₃): 7.28-7.13 (m, 4H, arom. H); 5.35 (m, 1H, olefin. H); 4.65 (d, 2H, benzyl. H); 3.85-4.00 (m, 8H, ethyleneketal-H); 3.73 (s, 1H, OH, H-D ex.); 0.85-2.6 (aliphatic H).

### 2.d) 18-(2-[4-Hydroxymethyl)-phenyl]-ethyl)-9β,10αa-pregna-4-ene-3,20-dione (No. 7)

The deketalization of compound XXIII-3 afforded the corresponding retroprogesterone derivative compound No. 7 according to step p in general SCHEME V.

To a solution of 1.19 g of the diketal XXIII-3 (2.22 mmol) in 65 ml of acetone, 4 ml of diluted (20%) sulfuric acid were added and stirred at RT for 5 h (TLC analysis: EtOAc/hexane 50:50 (vol.:vol.); educt R_{f} approx. 0.4; product R_{f} approx. 0.2). Work-up: The solvent acetone was removed under vacuum and the residue redissolved in a mixture of 250 ml DEE and 250 ml water. After thorough stirring, the phases were separated and the aq. phase was extracted 3× with DEE. The combined organic phases were washed (saturated NaHCO₃, H₂O, saturated NaCl) and dried (MgSO₄). After removal of the solvent under vacuum, 1.0 g of compound No. 7 was obtained as colorless frozen foam.

### 3.) 18-(2-[4-Formylphenyl]-ethyl)-9β,10α-pregna-4-ene-3,20-dione (No. 9)

18-(2-[4-Formylphenyl]-ethyl)-9β,10α-pregna-4-ene-3,20-dione (No. 9) was obtained by oxidation of the 18-(2-[4-Hydroxymethyl)-phenyl]-ethyl)-9β,10α-pregna-4-ene-3,20-dione (No. 7)

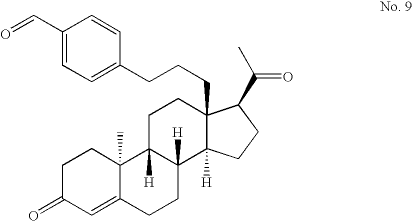

32 ml of water and 4 g of NaHCO₃ were added to a solution of 1.6 g of compound No. 7 (3.57 mmol) in 40 ml of DCM. The mixture is cooled down to 0-5° C. After addition of 17 mg of 2,2,6,6-tetramethylpiperidin-1-yloxy (TEMPO) (107 μmol), 2.2 ml of NaOCl solution (13%, 4.6 mmol) were added dropwise with vigorous stirring. The reaction mixture was stirred at 0 to 5° C. for 1 h (TLC analysis: EtOAc/hexane 50:50 (vol.:vol.); educt R_{f} approx. 0.3; product R_{f} approx. 0.5). Further portions of NaOCl-solution were added until complete conversion was obtained. Work-up: The phases were separated and the aq. phase was diluted with some water and extracted with DCM (2×40 ml). The combined organic phases were washed (H₂O) and dried (Na₂SO₄). After removal of the solvent under vacuum, 1.7 g residue was obtained, which was subjected to chromatography on silica gel (mobile solvent MTBE/hexane 50:50). Yield: 1.56 g of compound No. 9 were obtained as a colorless frozen foam.

### 4.) 18-(2-[4-Oximinoformylphenyl]-ethyl)-9β,10α-pregna-4-ene-3,20-dione (No. 1)

18-(2-[4-Oximinoformylphenyl]-ethyl)-9β,10α-pregna-4-ene-3,20-dione (No. 1) was obtained by conversion of the 18-(2-[4-Formylphenyl]-ethyl)-9β,10α-pregna-4-ene-3,20-dione (No. 9).

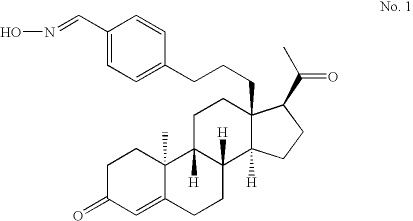

0.9 g of the aldehyde steroid (No. 9) (2.02 mmol) dissolved in 100 ml of ACN were mixed with 14 ml of acetate buffer (2.6 g NaOAc dissolved in 50 ml H₂O and adjusted with HOAc to pH 5.0) and 147 mg of NH₂OH.HCl (2.12 mmol), and stirred at RT for 3-5 h (TLC analysis: CHCl₃/MeOH 95:5 (vol.:vol.); educt R_{f} approx. 0.7; product R_{f} approx. 0.3). Work-up: The reaction mixture was poured into 200 ml of water and stirred thoroughly with 200 ml of DEE. The phases were separated and the aq. phase was extracted with DEE (3×100 ml). The combined organic phases were washed (H₂O) and dried (Na₂SO₄). After removal of the solvent under vacuum, 0.89 g of semi-crystalline residue was obtained which was subjected to chromatography on silica gel (mobile solvent CH₂Cl₂/MeOH 100:0 to 99.5:0.5). Fractions containing product were combined and crystallized from MTBE. Yield: 0.56 g of compound No. 1 was obtained as colorless crystals.

¹H-NMR (CDCl₃): 8.05 (s, 1H, CH-oxime); 7.40 (d, 2H, arom. H); 7.10 (d, 2H, arom. H); 5.65 (s, 1H, H-4); 2.5-1.05 (m, 26H, aliph. H); 2.15 (s, 3H, 21-CH₃); 1.30 (s, 3H, 19-CH₃).

### 5.) 18-(2-[4-N-Ethylcarbamoyl-oximino-formylphenyl]-ethyl)-9β,10α-pregna-4-ene-3,20-dione (No. 4)

18-(2-[4-N-Ethylcarbamoyl-oximino-formylphenyl]-ethyl)-9β,10α-pregna-4-ene-3,20-dione (No. 4) was obtained by reaction of the 18-(2-[4-Oximinoformylphenyl]-ethyl)-9β,10α-pregna-4-ene-3,20-dione (No. 1) with Ethylisocyanate.

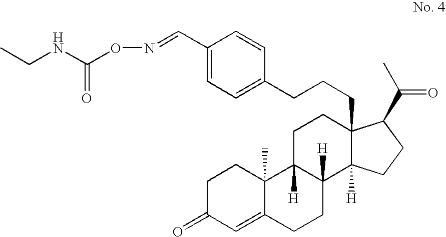

In a 25 ml Schlenk flask with reflux condenser, argon connection and magnetic stirrer, 0.312 g of the aldehyde oxime steroid (No. 1) (675 μmol) dissolved in 4 ml of toluene and 4 ml of ACN were provided in an inert argon atmosphere. Then, 9.4 μl of triethylamine (TEA) (68 μmol) and subsequently 105 μl, of ethyl isocyanate were added, and the mixture was stirred at 65° C. After intervals of 1 h, two further portions, each of 105 μl, of ethyl isocyanate, were added by syringe (in total 4.05 mmol ethyl isocyanate were added). The mixture was further stirred at 65° C. under argon. If necessary additional ethyl isocyanate/TEA was added until complete conversion was obtained (24-48 h) (TLC analysis: EtOAc/hexane 70:30 (vol.:vol.); educt R_{f} approx. 0.6; product R_{f} approx. 0.4). Work-up: The solvent was removed under vacuum and excess ethyl isocyanate/triethylamine was stripped out under high vacuum (1/4 h). The obtained residue (approx. 0.47 g) was purified by chromatography on silica gel (mobile solvent DCM/MeOH 100:0 to 99:1). Yield: 0.36 g of compound No. 4 were obtained as colorless frozen foam.

¹H-NMR (500 MHz, CDCl₃): 8.30 (s, 1H, CH-oxime); 7.57 (d, 2H, arom. H); 7.21 (d, 2H, arom. H); 6.23 (t, 1H, NH); 5.73 (s, 1H, H-4); 3.39 (m, 2H, CH₂); 2.63-1.1 (m, ˜26H, aliph. H); 2.2 (s, 3H, 21-CH₃); 1.25 (t, 3H, CH₃); 1.2 (s, 3H, 19-CH₃).

### 6.) 18-(2-[4-FormylPhenyl]-ethyl)-9β,10α-pregna-4,6-diene-3,20-dione (No. 10)

18-(2-[4-Formylphenyl]-ethyl)-9β,10α-pregna-4,6-diene-3,20-dione (No. 10) was obtained by dehydrogenation of the 18-(2-[4-Formylphenyl]-ethyl)-9β,10α-pregna-4-ene-3,20-dione (No. 9) according to step q in general SCHEME V.

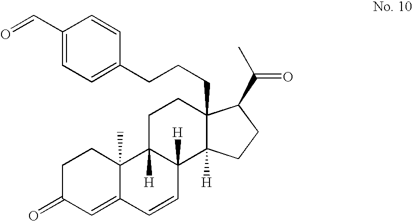

The reaction was performed as described above in example 1.e) with the following amounts of reagents:

15 ml of dioxane/HCl (approximately 170 mg HCl/ml)

a column (4×12 cm) packed with a slurry of aluminium oxide (neutral) in DEE

250 ml of ethereal hydrochloric acid (approx. 70 mg HCl/ml)

500 ml of DEE

250 mg of 18-(2-[4-formylphenyl]-ethyl)-retroprogesterone (No. 9) (560 μmol)

153 mg of DDQ (672 μmol)

2 ml of dioxane (anhydrous)

300 ml of DEE

21 of EtOAc/hexane 50:50

Yield: 0.23 g of crude product was obtained, which was dissolved in 2 ml of DCM. The mixture was diluted with 20 ml of MTBE, seeded and left to stand overnight at RT in order to crystallize. After suction filtration and washing with MTBE, 122 mg of compound No. 10 were obtained as yellowish crystals. The mother liquor still contained some product, which may be isolated by column chromatography.

¹H-NMR (500 MHz, CDCl₃): 10.00 (s, 1H, CHO); 7.83 (d, 2H, arom. H); 7.38 (d, 2H, arom. H); 6.16 (m, 2H, H-5, H-6); 5.66 (s, 1H, H-4); 3.00 (dd, 1H, H-17 (?); 2.75 (t, 2H, aliph. H); 2.57-2.39 (m, 3H, aliph. H); 2.20 (m, 1H, aliph. H); 2.10 (s, 3H, 21-CH3); 2.02-1.53 (m, 11H, aliph. H); 1.35-1.2 (m, 3H, aliph. H); 1.20 (s, 3H, 19-CH₃); 0.95 (m, 1H, aliph. H).

### 7.) 18-(2-[4-Oximinoformylphenyl]-ethyl)-9β,10α-pregna-4,6-diene-3,20-dione (No. 2)

18-(2-[4-Oximinoformylphenyl]-ethyl)-9β,10α-pregna-4,6-diene-3,20-dione (No. 2) was obtained by conversion of the 18-(2-[4-Formylphenyl]-ethyl)-9β,10α-pregna-4,6-diene-3,20-dione (No. 10).

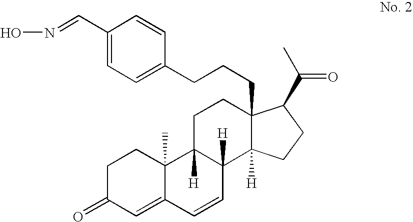

454 mg of the aldehyde steroid (No. 10) (1.03 mmol) dissolved in 130 ml of ACN were mixed with 76 mg of NH₂OH.HCl (1.09 mmol) dissolved in 5 ml acetate buffer (2.6 g of NaOAc in 50 ml H₂O adjusted with HOAc to pH 5.0). After rinsing with 2.2 ml of acetate buffer, the mixture was stirred at RT for 18 h (TLC analysis: CHCl₃/MeOH 95:5 (vol.:vol.); educt R_{f} approx. 0.7; product R_{f} approx. 0.3). Work-up: The solvent was removed under vacuum down to a residue of about 40 ml, which was diluted with 600 ml of water and stirred thoroughly with 200 ml of MTBE. The phases were separated and the aq. phase was extracted with DEE (2×150 ml). The combined organic phases were washed (H₂O) and dried (Na₂SO₄). After removal of the solvent under vacuum, 0.55 g of residue was obtained which was subjected to chromatography on silica gel (mobile solvent CH₂Cl₂/MeOH 100:0 to 95:5). Fractions containing product were combined and crystallized from MTBE/hexane. After suction filtration and drying, 223 mg of compound No. 2 was obtained as colorless crystals.

¹H-NMR (500 MHz, CDCl₃): 8.05 (s, 1H, CH-oxime); 7.45 (d, 2H, arom. H); 7.15 (d, 2H, arom. H); 6.08 (m, 2H, H5, H6); 5.60 (s, 1H, H-4); 3.15 (s, 1H, OH(?); 2.95 (m, 1H, aliphat. H); 2.6-0.9 (m, 21H, aliph. H); 2.0 (s, 3H, 21-CH₃); 1.15 (s, 3H, 19-CH₃).

### 8.) 18-(2-[4-N-Ethylcarbamoyl-oximino-formylphenyl]-ethyl)-9β,10α-pregna-4,6-diene-3,20-dione (No. 5)

18-(2-[4-N-Ethylcarbamoyl-oximino-formylphenyl]-ethyl)-9β,10α-pregna-4,6-diene-3,20-dione (No. 5) was obtained by reaction of the 18-(2-[4-Oximinoformylphenyl]-ethyl)-9β,10α-pregna-4,6-diene-3,20-dione (No. 2) with ethyl isocyanate.

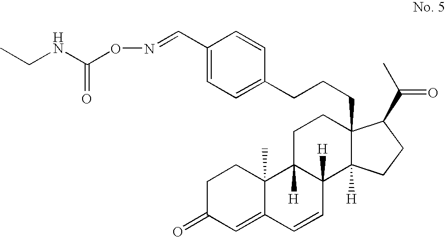

In a 25 ml Schlenk flask with reflux condenser, argon connection and magnetic stirrer, 173 mg of aldehyde oxime steroid (No. 2) (376 μmol) dissolved in 2.4 ml of toluene and 2.4 ml of ACN, were mixed with 10.5 μl of TEA (75 μmol). Then, 178 μl of ethyl isocyanate were added and the reaction mixture was stirred under argon at 65° C. for 18 h (TLC analysis: EtOAc/hexane 70:30 (vol.:vol.); educt R_{f} approx. 0.6; product R_{f} approx. 0.5). Work-up: The solvent was removed under vacuum and excess ethyl isocyanate was stripped out under high vacuum (¼ h). The residue (0.2 g) was purified by chromatography on silica gel (mobile solvent MTBE/hexane 80:20 to 95:5). Yield: 165 mg of compound No. 5 were obtained as yellowish frozen foam.

¹H-NMR (500 MHz, CDCl₃): 8.25 (s, 1H, CH-oxime); 7.55 (d, 2H, arom. H); 7.2 (d, 2H, arom. H); 6.1 (m, 2H, H-5, H-6); 5.60 (s, 1H, H-4); 3.33 (m, 2H, CH₂); 2.92 (d, 1H, aliph. H); 2.65-0.9 (m, 23H, 22 aliph. H, 1×OH); 2.02 (s, 3H, 21-CH₃); 1.15 (t, 3H, CH₃); 1.12 (s, 3H, 19-CH₃).

### 9.) 18-[2-(4-Formic acid-phenyl)-ethyl]-(9β,10α)-pregna-4-ene-3,20-dione (No. 14)

This compound No. 14 of the invention was obtained by reactions as displayed within general reaction SCHEMES II (step h, Wittig addition), IV and V. The reactions were carried out as described above under example 2.).

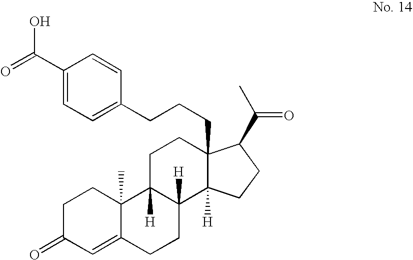

### 9.a) 18-(2-[4-Methoxycarbonyl-phenyl]-vinyl)-9β,10α-pregna-5-ene-3,20-diethylenedioxyketal XVII-4

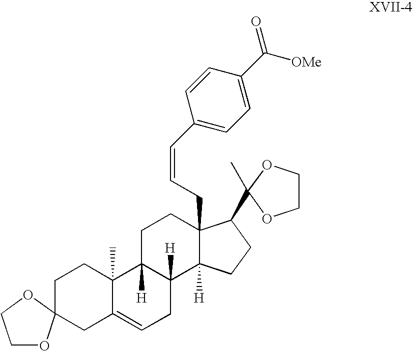

This intermediate compound was prepared by a Wittig addition using the following phosphonium salt as starting reagent:

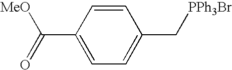

In a 500 ml RB flask equipped with a magnetic stirrer, septum and bubbler, argon was flushed in and 6.56 g dry phosphonium salt (13.4 mmol) was suspended in 250 ml toluene (abs). Approximately 30 ml toluene was distilled off in vacuum under inert atmosphere of argon. Then, KOtBu (1.5 g, 13.4 mmol) was added and the reaction mixture was stirred at RT (1 h). Stirring was discontinued to let the contents settle down. In a separate, properly dried RB flask equipped with a septum and magnetic stirrer, 2.3 g crude 18-formyl-Δ-5-diketal (XVI-H) was dissolved in 50 ml toluene under argon atmosphere. The freshly prepared, clear ylid-solution was added through cannula. The reaction mixture was stirred under a slow stream of argon overnight at 65° C. TLC control showed only incomplete conversion which could be improved slightly by further stirring at 90° C. for another 1.5 h (TLC analysis: EtOAc/hexane 40:60 (vol.:vol.); educt R_{f} approx. 0.3; product R_{f} approx. 0.4). Work-up: The reaction mixture was poured over ½ l saturated aq. sodium bicarbonate and vigorously stirred. The layers were separated after settling down and aq. layer was extracted toluene (2×200 ml). The combined organic extracts were washed with H₂O, and dried over anhydrous Na₂SO₄. Removal of solvent under vacuum and column chromatography over silica gel using EtOAc/hexane (20:80) yielded 0.97 g of compound XVII-4 as colorless frozen foam.

¹H-NMR: 7.98 (d, 2H, arom. H); 7.37 (d, 2H, arom. H); 6.7-6.1 (4 peaks, 2H, olefin-H, ˜2:1 cis/trans); 5.36 (m, 1H, H-6); 4.1-3.9 (m, 8H, ethylene-H); 3.9 (s, 3H, OMe); 2.6-1.15 (aliph.-H).

### 9.b) 18-(2-[4-Methoxycarbonyl-phenyl]-ethyl)-9β,10α-Pregna-5-ene-3,20-diethylenedioxyketal XXIII-4

The next reaction step starting from compound XVII-4 was the reduction of the unsaturated side chain to produce the corresponding 18-(2-[4-Methoxycarbonyl-phenyl]-ethyl)-(9β,10α)-pregna-5-ene-3,20-diketal XXIII-4 according to step o in general SCHEME IV.

In a 250 ml Schlenk flask equipped with magnetic stirrer, septum, three-way tap with hydrogen feed (gas burette) and argon flask, 35 mg of catalyst (palladium on calcium carbonate (5%)) were suspended in 20 ml ethanol. The flask was repeatedly evacuated and flushed with argon (3×), and then 3× evacuated and filled with hydrogen. Then the catalyst was hydrogenated under vigorous stirring. A solution of 0.84 g of the Wittig adduct (XVII-4) (1.51 mmol) in 20 ml of toluene was degased with argon and added into the flask by syringe. After rinsing the latter with 2.5 ml of toluene, the educt was hydrogenated under vigorous stirring for 3 h. (TLC analysis: EtOAc/hexane 30:70 (vol.:vol.); educt R_{f} approx. 0.3; product R_{f} approx. 0.35. Work-up: The flask was evacuated and flushed with argon (3×). The reaction mixture was suction filtered through a bed of diatomaceous earth and rewashed with some toluene/ethanol. The filtrate was concentrated under vacuum. Yield: 0.82 g of (XXIII-4) was obtained as colorless frozen foam.

### 9.c) 18-(2-[4-Formic acid-Phenyl]-ethyl)-9β,10α-pregna-5-ene-3,20-diethylenedioxyketal XXIII-5

The next reaction step starting from compound XXIII-4 was the removal of the protection group in the side chain to produce the corresponding 18-(2-[4-Formic acid-phenyl]-ethyl) steroid XXIII-5.

In a 250 ml round-bottomed flask equipped with a magnetic stirrer, 0.8 g ester (XXIII-4) (1.42 mmol) was dissolved in 30 ml dioxane. After addition of 20 ml MeOH, 119 mg LiOH.H₂O (2.83 mmol) dissolved in 8 ml water were added dropwise under stirring at 40° C. The stirring was continued under argon for 30 h (TLC analysis EtOAc/hexane 40:60 (vol.:vol.); educt R_{f} approx. 0.5; product R_{f} approx. 0.3). Work-up: The reaction mixture was roughly neutralised with acetic acid (170 mg in 2 ml dioxane) and the solvents were evaporated under reduced pressure. The residue was repeatedly taken in dioxane and dried under vacuum (2×20 ml). The crude product thus obtained was used for deketalisation without further purification.

### 9.d) 18-(2-[4-Formic acid-phenyl]-ethyl)-9β,10α-pregna-4-ene-3,20-dione (No. 14)

The deketalization of compound XXIII-5 afforded the corresponding retroprogesterone derivative compound No. 14 according to step p in general SCHEME V. The reaction was carried out as described above in example 2.d) with the following amounts of reagents:

0.8 g 18-(2-[4-Formic acid-phenyl]-ethyl)-Δ-5-retroprogesterone-3,20-diethylendioxyketal (XXIII-5) (crude, about 1.4 mmol); 100 ml acetone; 5 ml sulfuric acid (20%)

TLC analysis: EtOAc/hexane 40:60 (vol.:vol.) educt R_{f} approx. 0.3; product R_{f} approx. 0.15. Work-up: 4 g NaHCO₃ and 10 ml of water were added to the reaction mixture, and stirring was continued for 5 min followed by concentration under vacuum to 10 ml. The reaction mixture was then diluted with DCM und water (100 ml +100 ml). The pH was adjusted to 1 by addition of 5% hydrochloric acid. After stirring well the phases were separated. The aq. phase was extracted with DCM (2×50 ml). The combined organic extracts were washed with H₂O and dried (Na₂SO₄). After removal of the solvents under vacuum, 0.70 g of compound No. 14 was obtained as yellowish frozen foam.

¹H-NMR: 8.0 (d, 2H, arom. H); 7.23 (d, 2H, arom. H); 5.74 (s, 1H, H-4); 2.2 (s, 3H, Me-21); 1.4 (s, 3H, Me-19); 2.7-1.2 (aliph. H).

### 10.) 18-[2-(4-Formic acid-phenyl)-ethyl]-9β,10α-pregna-4,6-diene-3,20-dione (No. 15)

18-(2-[4-Formic acid-phenyl]-ethyl)-9β,10α-pregna-4,6-diene-3,20-dione (No. 15) was obtained by dehydrogenation of the 18-(2-[4-Formic acid-phenyl]-ethyl)-9β,10α-pregna-4-ene-3,20-dione (No. 14) according to step q in general SCHEME V.

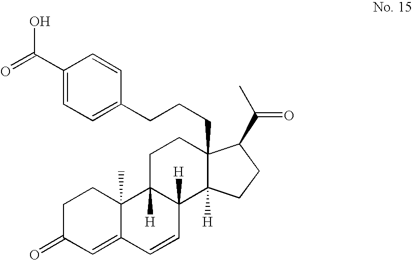

300 mg 18-(2-[4-Formic acid-phenyl]-ethyl)-retroprogesterone (No. 14) (649 μmol) was dissolved in 5 ml dioxane under argon. 7.5 ml dioxane/HCl was added (containing 100 mg HCl/ml). 162 mg DDQ (713 μmol) in 10 ml dioxane was added to the reaction mixture and stirred (10 min), followed by treatment with a mixture of 3.4 g sodium acetate, 25 ml water and 25 ml DCM under continued stirring. The phases were separated, the aq. phase acidified with 5% hydrochloric acid (pH˜2) and extracted again (DCM, 3×10 ml). The combined organic extracts were washed with water and dried over Na₂SO₄. The solvent was evaporated under vacuum, the residue subjected to column chromatography on silica gel (solvent system DCM/MeOH 2%) to yield 0.21 g compound No. 15 as yellow frozen foam.

TLC analysis: DCM/MeOH 90:10; educt/ product R_{f} approx. 0.3.

¹H-NMR: 8.0 (d, 2H, arom. H); 7.24 (d, 2H, arom. H); 6.16 (m, 2H, H-5, H-6); 5.7 (s, 1H, H-4); 2.2 (s, 3H, Me-21); 1.3 (s, 3H, Me-19); 2.75-1.2 (aliph. H).

### 11.) 18-[2-(4-Formamido-phenyl)-ethyl]-(9β,10α)-pregna-4,6-diene-3,20-dione (No. 13)

The 18-(2-[4-Formic acid-phenyl]-ethyl)-9β,10α-pregna-4-ene-3,20-dione (No. 14) was converted into the corresponding amide No. 13 by nucleophilic substitution.

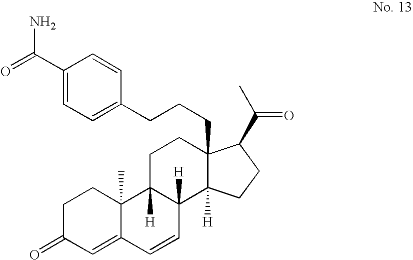

In a 25 ml pear-shaped flask equipped with magnetic stirrer and ice bath, 215 mg 18-(2-[4-Formic acid-phenyl]-ethyl)-dydrogesterone (467 μmol) were added to 2.2 ml THF and 2.2 ml ACN. After addition of 66.2 mg Hydroxybenzotriazole-Hydrate (HOBT) (490 μmol), the flask was cooled to 0° C.; then 101 mg Dicyclohexylcarbodiimide (DCC) (490 μmol), dissolved in 0.25 ml THF, were added followed by the addition of 3.8 ml ACN/NH₃ (6.3 mg NH₃/ml; 1.4 mmol NH₃). The stirring was continued overnight at RT (TLC analysis: EtOAc/hexane 80:20+3 drops HAc/5ml; educt R_{f} approx. 0.6; product R_{f} approx. 0.2). Work-up: The reaction mixture was filtered through diatomaceous earth and washed with a small quantity of THF. The solid was discarded. The solvent of the filtrate was removed under vacuum and the remaining residue was dissolved in 20 ml DCM and subjected to repeated washings (1% HCl, 1% NaOH und H₂O). The combined organic layers were dried over anhydrous MgSO₄ The solvents were evaporated under vacuum, chromatographed over silicagel column with EtOAc/hexane (80:20) as solvent to yield 0.193 g of compound No. 13 as frothy solid.

¹H-NMR: 7.3 (d, 2H, arom. H), 7.22 (d, 2H, arom. H); 6.17 (m, 2H, H-5, H-6); 6.05 (br.s, 2H, NH2); 5.7 (s, 1H, H-4); 2.7-1.2 (aliph. H).

### 12.) Introduction of a Hydroxyl Group in C17 Alpha Position of the Steroidal Core

The introduction of a —OH group in C17 alpha position of compound No. 7 was performed as displayed in general reaction SCHEME VII (and according to reactions as displayed in U.S. Pat. No. 3,555,053 and by Halkes & van Moorselaar [1969].

### 12.a) 18-(2-[4-TBDPS-oxymethyl-phenyl]-ethyl)-(9β,10α)-pregna-4-ene-3,20-dione (XXVI-1)

At first, the free hydroxyl group in the side chain of compound No. 7 has to be protected by a suitable protective group as displayed in the following reaction scheme:

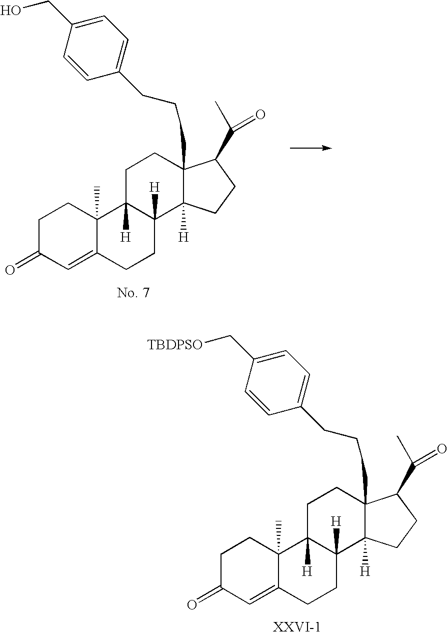

7.35 g of 18-(2-[4-(Hydroxymethyl)-phenyl]-ethyl)-retroprogesterone (No. 7) (16.4 mmol) were dissolved in 125 ml dry DMF; then, imidazole (2.0 g, 29.5 mmol) was added, and the contents were cooled to 0-5° C. under stirring. 7.2 g tert.-butyldiphenylsilylchloride (TBDPS-Cl) (26.2 mmol) were added and stirring was continued overnight (0° C. RT). TLC analysis: EtOAc/hexane 50:50 (vol.:vol.); educt R_{f} approx. 0.25; product R_{f} approx. 0.6. Work-up: The reaction mixture was poured into 0.5 l H₂O and 0.5 l DEE under vigorous stirring. The phases were separated and the aq. phase was extracted with DEE (2×200 ml).The combined organic extracts were washed with H₂O and saturated NaCl-solution, and dried over anhydrous Na₂SO₄. The residue obtained after removal of the solvents under vacuum was subjected to column chromatography on silica gel while eluting with EtOAc/hexane (30:70) to give 10.7 g of product (XXVI-1) as colorless frothy solid.

¹H-NMR (CDCl₃); 7.68 (d, 4H, arom. H); 7.4 (d, 2H, arom. H); 7.35 (m, 4H, arom, H), 7.24 (d, 2H, arom. H); 7.10 (d, 2H, arom. H); 5.72 (s, 1H, H-4); 2.5-1.2 (m, 26, aliph. H); 2.2 (s, 3H, 21-CH₃); 1.4 (s, 3H, 19-CH₃); 1.1 (s, 9H, t-Bu)

### 12.b) 18-(2-[4-TBDPS-oxymethyl-phenyl]-ethyl)-(9β,10α)-pregna-4-ene-3,20-diol (XXXI-1)

The product XXVI-1 obtained is then reduced to the corresponding 3,20-diol of formula XXXI-1:

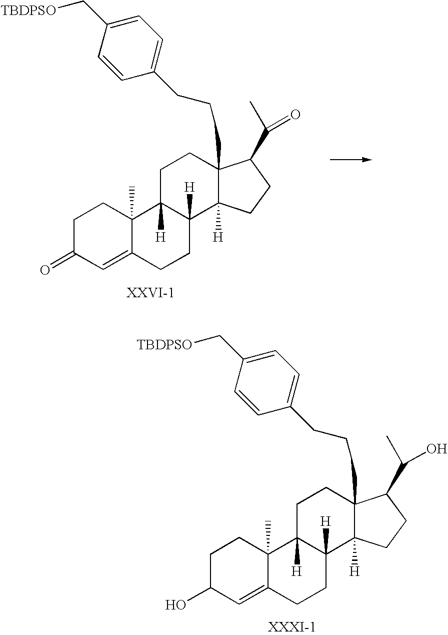

A 500 ml 3-necked round bottom flask equipped with a magnetic stirrer, a thermometer and a dropping funnel was placed in an ice-salt bath and flushed with argon. Under positive argon-pressure, 1.8 g LAH (48 mmol) were placed in the flask, cooled to −10° C. and 100 ml THF were added under vigorous stirring. 10.7 g 18-(2-[4-(TBDPS-oxymethyl)-phenyl]-ethyl)-retroprogesterone (XXVI-1) (16 mmol), dissolved in 100 ml abs THF, were added dropwise at −10° C. to −5° C. (¼ h) and stirred at −10° C. for 30 min (TLC analysis: EtOAc/hexane 30:70 (vol.:vol.); educt R_{f} approx. 0.25; product R_{f} approx. 0.2). Work-up: 2 ml of water were added dropwise to the reaction mixture under thorough cooling, followed by 2 ml of 15% aq. NaOH und 6 ml of water. The reaction mixture was brought to RT slowly under stirring, and 0.3 l DEE and 200 ml 15% aq. NaOH solution were added. The phases were separated and the aq. phase was extracted with DEE (200 ml). The aq. phase was treated with 15% aq. NaOH (15 ml), 10 g potassium sodium tartrate, and 200 ml DEE and stirred for about ½ h. After separation the organic extracts were combined, washed (H₂O, sat. aq NaCl) and dried over Na₂SO₄. The solvent was removed under vacuum to give 11.3 g of (XXXI-1) as a colorless frothy solid (crude product).

### 12.c) 18-(2-[4-TBDPS-oxymethyl-phenyl]-ethyl)-(9β,10α)-pregna-4-ene-3-one-20-ol (XXXII-1)

The 3-hydroxy group of intermediate compound XXXI-1 is then selectively re-oxidized to yield the compound XXXII-1 according to the following reaction scheme:

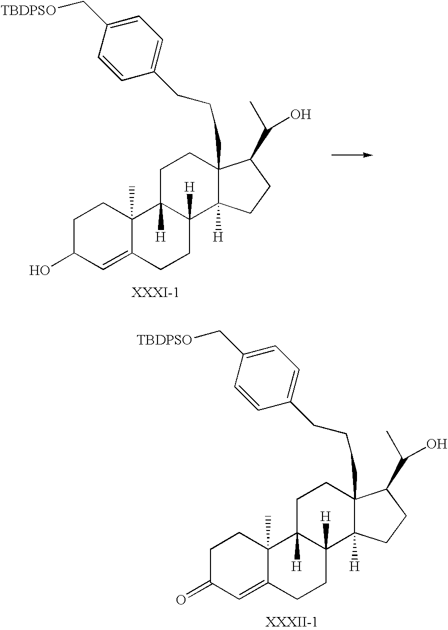

To 11.3 g crude 18-(2-[4-(TBDPS-oxymethyl)-phenyl]-ethyl)-9β,10α-pregn-4-en-3,20-diol (XXXI-1) (˜15.6 mmol) dissolved in 150 ml DCM were added 25 g MnO₂ (Aldrich, particle size <5 μm) and the mixture was stirred vigorously overnight. TLC analysis: DCM/Et₂O 5:0.5 (vol.:vol.); educt R_{f} approx. 0.3; product R_{f} approx. 0.4. Further portions of MnO₂ (3-5 g) were added to the reaction mixture under stirring to finish the reaction. Work-up: The contents were filtered and washed with a little amount of DCM. The solid cake was treated and washed with DCM repeatedly (3×150 ml) under refluxing (2 min) and stirring. After cooling to RT under stirring, the solids were filtered. The filtrates were combined and the solvent removed under vacuum to give 10.4 g of a residue which was purified by column chromatography on silica gel using DCM/MeCN as solvent. The fractions containing product were combined. Removal of solvent under vacuum gave 8.6 g of (XXXII-1) as frothy solid.

¹H-NMR (CDCl₃): 7.7 (d, 4H, arom. H); 7.35 (m, 6H, arom. H); 7.25 (d, 2H, arom. H); 7.15 (d, 2H, arom. H); 5.7 (s, 1H, H-4); 4.7 (s, 2H, benzyl H); 3.65 (m, 1H, H-20); 2.6 (t, 2H); 2.4-0.9 (m, 24H, aliph. H); 1.4 (H-D-exchangeable), 1.35 (s, 3H, 19-CH₃); 1.15 (d, 3H, 21-Me); 1.1 (s, 9H, t-Bu)

### 12.d) 18-(2-[4-TBDPS-oxymethyl-phenyl]-ethyl)-9β,10α-pregna-4,17(20)-diene-3-one (XXXIII-1)

Then, compound XXXII-1 was further dehydrated by tosylation with tosyl chloride in pyridine and subsequent treatment of the generated tosylate with boiling pyridine in order to afford the 17,20 unsaturated derivative of formula XXXIII-1 in a mixture of cis and trans isomers.

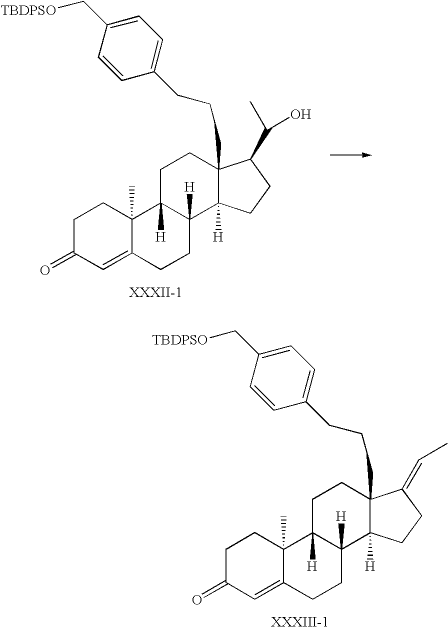

8.6 g 18-(2-[4-(TBDPS-oxymethyl)-phenyl]-ethyl)-9β,10α-pregn-3-on-4-ene-20-ol (XXXII-1) (12.5 mmol) were dissolved in 120 ml pyridine, and 4.76 g pTosCl (25 mmol) and 80 mg DMAP were added. The mixture was stirred at RT for 48 h under argon. The temperature was raised to 60° C. (bath) and stirring was continued for further 18 h. TCL analysis: EtOAc/hexane 30:70 (vol.:vol.); educt R_{f} approx. 0.25; intermediate (probably 20-tosylate) R_{f} approx. 0.3; product R_{f} approx. 0.6. Work-up: The solvent was evaporated under vacuum. The residue was washed with 75 ml of water, diluted with 150 ml DEE and stirred well. Three phases were observed. The organic phase was separated. The middle oily phase was combined to the aq. phase and extracted with DEE (3×200 ml). The combined organic phase and extracts were washed with 4×150 ml 3% aq. KHSO₄, 1× with 150 ml saturated aq. NaHCO₃ solution and 1× with 150 ml saturated aq. NaCl solution. The washings were repeatedly extracted and purified as above. The combined organic extracts were dried over anhydrous sodium sulfate. After removing the solvent under vacuum, the residue was subjected to column chromatography (solvent system MTBE/hexane 10:90) to give 6.39 g of (XXXIII-1) as colorless frothy.

### 12.e) 18-(2-[4-TBDPS-oxymethyl-phenyl]-ethyl)-17α-hydroxy-9β,10α-pregna-4-ene-3,20-dione (XXXIV-1)

The obtained compound XXXIII-1 was then oxygenated using NMMO as stochiometric oxidizing agent and additional hydrogen peroxide in the presence of a catalytic amount of osmium tetroxide to yield compound XXXIV-1.

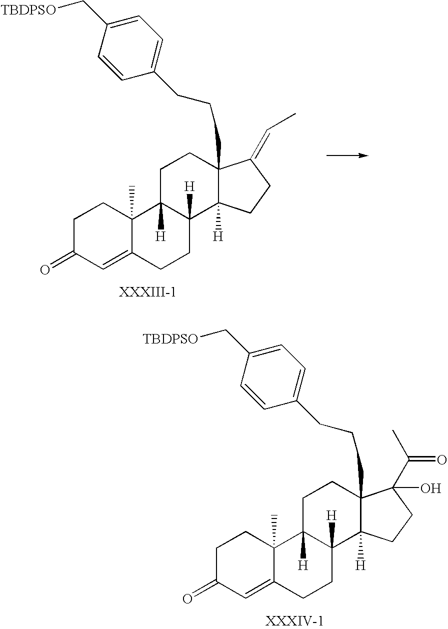

To 6.39 g 18-(2-[4-(TBDPS-oxymethyl)-phenyl]-ethyl)-9β,10α-pregna-4,17(20)-dien-3-on (XXXIII-1) (9.52 mmol) dissolved in 400 ml tert.-Butanol, 5.4 ml Pyridine and 1.9 ml Osmiumtetroxide solution (4% in tert.-Butanol; 238 μmol) were added. After stirring for 20 min at RT, 2.79 g NMMO (23.8 mmol), dissolved in 50 ml tert.-Butanol, and 2.04 ml 35% aq. H₂O₂ (23.8 mmol) were added with stirring at RT for 54 h. TLC analysis: EtOAc/hexane 40:60 (vol.:vol.); educt R_{f} approx. 0.75; product R_{f} approx. 0.35. Work-up: The reaction mixture was worked up by addition of 120 ml of H₂O and 12 g Na₂S₂O₄ followed by vigorous stirring (10 min). After addition of 50 ml of aq. NaHSO₃ solution (35%), stirring was continued for about 30 min. The reaction mixture was extracted with DEE (5×150 ml). The combined organic extracts were washed with 2×200 ml aq. KHSO₄ (3%), 200 ml saturated aq. NaHCO₃ and saturated aq. NaCl. All washings were extracted again. The combined organic extracts were dried over anhydrous Na₂SO₄. The residue obtained after removing the solvent was chromatographed over a column of silica gel to give 3.56 g of (XXXIV-1) as colorless frothy solid.

¹H-NMR (CDCl₃): 7.62 (d, 4H, arom. H); 7.3 (m, 6-H, arom. H); 7.25 (d, 2H, arom. H); 7.03 (d, 2H, arom. H); 5.65 (s, 1H, H-4); 4.65 (s, 2H, benzyl-H); 2.7-0.8 (m, 26H, aliphatic); 2.2 (s, 3H, 21-Me); 1.35 (s, 3H, 19-Me); 1.0 (s, 9H, t-Bu).

### 13.) 18-(2-[4-TBDPS-oxymethyl-phenyl]-ethyl)-3,20-dioxo-(9β,10α)-pregna-4-ene-17-yl-carbonic acid ethyl ester XXXVII-1

The compound XXXIV-1 obtained in example 12e) was then carboxylated in the presence of ethyl-jodide and Ag₂CO₃ to yield the 17-carbonic acid ethyl ester compound with an average yield of 70%. The reaction was performed according to general SCHEME VIII:

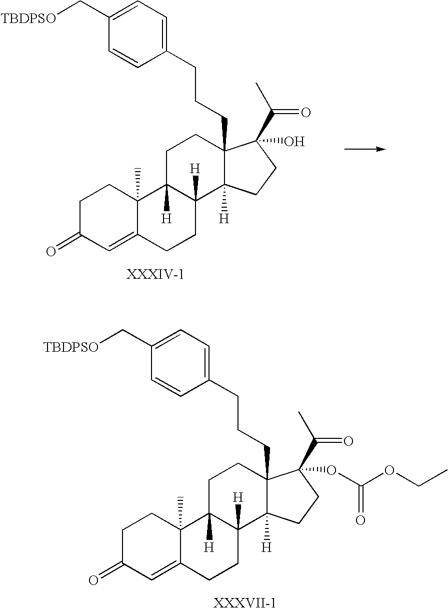

To 2.3 g 18-(2-[4-(TBDPS-oxymethyl)-phenyl]-ethyl)-17-hydroxy-9β,10α-pregna-4-ene-3,20-dione (XXXIV-1) (3.27 mmol) dissolved in 55 ml DMF, 5.3 ml ethyl-jodide (156 g/mol; 20 eq.) and 18 g Ag₂CO₃ (275.8 g/mol; 20 eq.) were added, and the mixture was stirred at 70° C. (1 h). TCL analysis: EtOAc/hexane 40:60 (v:v); educt R_{f} approx. 0.4; product R_{f} approx. 0.5. Further 1.3 ml ethyljodide and 4.6 g Ag₂CO₃ were added and stirring was continued for 1 h. The mixture was cooled down and the solid (Ag-salts) was removed. After removal of the solvent in the remaining filtrate under vacuum, the residue was subjected to column chromatography (silica gel, solvent system EtOAc/hexane 20:80) to give 1.8 g of (XXXVII-1) as colorless frothy.

¹H-NMR (500 MHz, CDCl₃): δ 7.61 (d, 4H, arom. —H); 7.36-7.28 (m, 6H, arom.-H); 7.18 (m, 2H, arom.-H); 7.04 (m, 2H, arom.-H); 5.66 (s, 1H, H-4); 4.66 (s, 2H, benzyl-H); 4.10 (m, 2H, CH₂ (OEt)); 2.95 (m, 1H); 2.1 (s, 3H, 21-CH₃); 1.4 (s, 3H, 19-CH₃); 1.25 (t, 3H, CH₃ (OEt)); 1.0 (s, 9H, tBu); 2.55-0.8 (m, aliph.-H).

### 14.) 18-(2-[4-Hydroxymethyl-phenyl]-ethyl)-3,20-dioxo-(9β,10α)-pregna-4-ene-17-yl-carbonic acid ethyl ester (No. 8). 18-[2-(4-Formyl-phenyl)-ethyl]-3,20-dioxo-(9β,10α)-pregna-4-ene-17-yl-carbonic acid ethyl ester (No. 11) and 18-[2-(4-Formyl-phenyl)-ethyl]-3,20-dioxo-(9β,10α)-pregna-4,6-diene-17-yl-carbonic acid ethyl ester (No. 12)

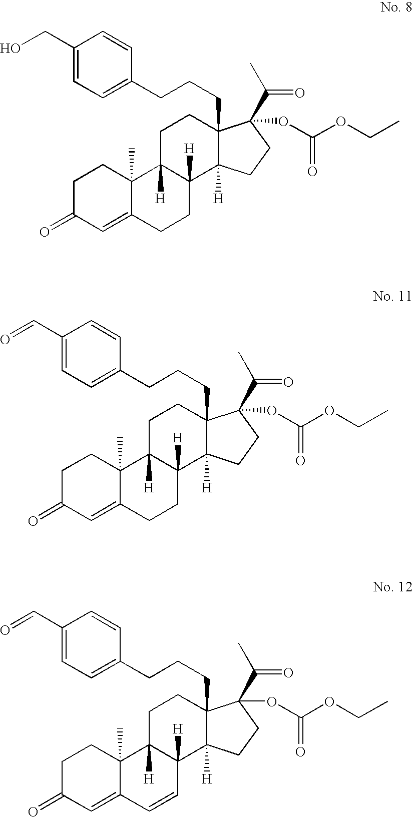

The compound XXXVII-1 obtained in example 13.) was then desilylated with TBAF to produce the corresponding 18-(2-[4-Hydroxymethyl-phenyl]-ethyl)-3,20-dioxo-(9β,10α)-pregna-4-ene-17-yl-carbonic acid ethyl ester (No. 8), which was subsequently oxidised with NaOCl to yield the aldehyde (No. 11). DDQ dehydrogenation of the aldyhyde (No. 11) to yield the corresponding dydrogesterone derivative (No. 12) was performed in three batches at 250 mg scale. An average yield of approximately 60% was achieved. The reactions were performed as described above in examples 2c, 3, and 1e, respectively.

### 18-(2-[4-Hydroxymethyl-phenyl]-ethyl)-3,20-dioxo-(9β,10α)-pregna-4-ene-17-yl-carbonic acid ethyl ester (No. 8)

¹H-NMR (500 MHz, CDCl₃): δ 7.2 (d, 2H, arom.-H); 7.1 (d, 2H, arom.-H); 5.6 (s, 1H, H-4); 4.6 (s, 2H, benzyl-H); 4.1 (m, 2H, CH₂ (OEt)); 2.9 (m, 1H); 2.1 (s, 3H, 21-CH₃); 1.4 (s, 3H, 19-CH₃); 1.25 (t, 3H, CH₃ (OEt)); 2.5-1.0 (m, aliph.-H).

### 18-[2-(4-Formyl-phenyl)-ethyl]-3,20-dioxo-(9β,10α)-pregna-4-ene-17-yl-carbonic acid ethyl ester (No. 11)

Educt: 0.81 g of compound No. 8 (1.5 mmol)

TLC analysis: EtOAc/hexane 50:50 (vol.:vol.); educt R_{f} approx. 0.4; product R_{f} approx. 0.6

Product: 780 mg of compound No. 11 as colorless frothy

### 18-[2-(4-Formyl-phenyl)-ethyl]-3,20-dioxo-(9β,10α)-pregna-4,6-diene-17-yl-carbonic acid ethyl ester (No. 12)

¹H-NMR (500 MHz, CDCl₃): δ 9.9 (s, 1H, CHO); 7.7 (d, 2H, arom.-H); 7.25 (d, 2H, arom.-H); 6.1 (m, 2H, H-5, H-6); 5.6 (s, 1H, H-4); 4.1 (m, 2H, CH₂ (OEt)); 2.95 (m, 1H); 2.1 (s, 3H, 21-CH₃); 1.35 (s, 3H, 19-CH₃); 1.2 (t, 3H, CH₃ (OEt)); 2.6-0.8 (m, aliph.-H).

### 15.) 18-[2-(4-Oximino-formylphenyl)-ethyl]-3,20-dioxo-((9β,10α)-pregna-4,6-diene-17-yl-carbonic acid ethyl ester (No. 3) and 18-[2-(4-N-Ethylcarbamoyl-oximino-formylphenyl)-ethyl]-3,20-dioxo-((9β,10α)-pregna-4,6-diene-17-yl-carbonic acid ethyl ester (No. 6)

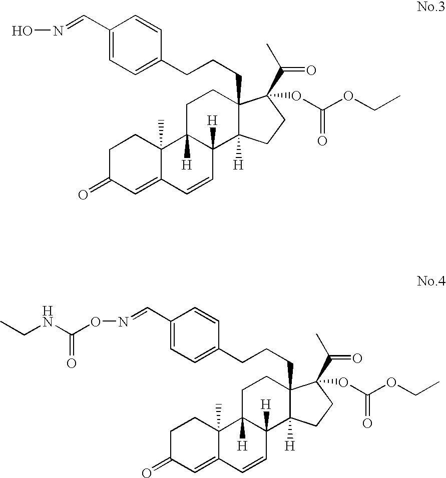

The aldehyde 18-[2-(4-Formyl-phenyl)-ethyl]-3,20-dioxo-(9β,10α)-pregna-4,6-diene-17-yl-carbonic acid ethyl ester (No. 12) was then transformed into the corresponding oxime derivative (compound No. 3) by a reaction as described in example 7 and purified by chromatography. Then, the purified compound No. 3 was further reacted to yield the carbamoyl oxime compound No. 6 in a reaction as described in examples 5 and 8.

### 18-[2-(4-Oximino-formylPhenyl)-ethyl]-3,20-dioxo-((9β,10α)-pregna-4,6-diene-17-yl-carbonic acid ethyl ester (No. 3)

Educt: 480 mg of compound No. 12 (901 μmol)

TLC analysis: chloroform/MeOH 95:5 (vol.:vol.); educt R_{f} approx. 0.7; product R_{f} approx. 0.3

Product: 376 mg of compound No. 3 as yellowish frothy

### 18-[2-(4-N-Ethylcarbamoyl-oximino-formylphenyl)-ethyl]-3,20-dioxo -((9β,10α)-pregna-4,6-diene-17-yl-carbonic acid ethyl ester (No. 6)

¹H-NMR (500 MHz, CDCl₃): δ 8.3 (s, 1H, CH═N); 7.6 (d, 2H, arom.-H); 7.2 (d, 2H, arom.-H); 6.2 (m, 3H, H-5, H-6, NH); 5.7 (s, 1H, H-4); 4.15 (m, 2H, CH₂ (OEt)); 3.4 (m, 2H, CH₂ (NEt)); 3.0 (m, 1H); 2.15 (s, 3H, 21-CH₃); 1.4 (s, 3H, 19-CH₃); 1.3 (t, 3H, OCH2CH₃); 1.25 (t, 3H, NCH_{2CH3}); 2.6-1.1 (m, aliph.-H).

### 16.) The Following Further Illustrative Compounds were Prepared According to the General Procedure as Displayed within General Reaction SCHEMES II, IV, V and VI, Respectively

The synthesis of the following retroprogesterone derivatives No. 16, 18, 20, 22, 24, 26, 28, 30 and 32 was achieved starting from intermediate compound XVI-H according to the reactions as described for Example 2 and Example 9 using the corresponding Wittig reagent Ph₃P═CH—Ar or Ph₃P═CH-HetAr (reactions 2a and 9a, the Wittig reaction, correspond to step h of the general schemes; reactions 2b and 9b, achieving the reduction of the unsaturated side chain, correspond to step o in the general schemes; reactions 2d and 9d, the deketalization of C18 substituted compound, deliver the corresponding retroprogesterone derivative and correspond to step p in the general schemes). The dehydrogenation of the obtained retroprogesterone compounds carried out as described for Examples 1e and 6 delivers the corresponding dydrogesterone derivatives No. 17, 19, 21, 23, 25, 27, 29, 31 and 33 (this reaction corresponds to step q in the general schemes).

### 18-[2-Phenyl-ethyl]-(9β,10α)-pregna-4-ene-3,20-dione (No. 16)

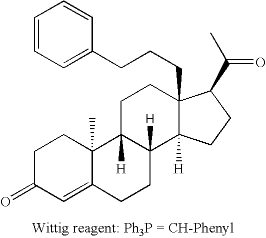

### 18-[2-Phenyl-ethyl]-(9β,10α)-pregna-4,6-diene-3,20-dione (No. 17)

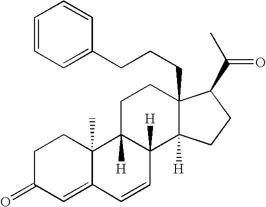

¹H-NMR (500 MHz, CDCl₃): δ 7.25-7.1 (m, 5H, arom. H); 6.1 (m, 2H, H-5, H-6); 5.6 (s, 1H, H-4); 2.9 (m, 1H); 2.6 (m, 2H, allyl. H); 2.5-0.9 (m, aliph. H); 2.0 (s, 3H, 21-CH₃); 1.1 (s; 3H, 19-CH₃).

### 18-[2-benzo[1,3]dioxol-5-yl-ethyl]-(9β,10α)-pregna-4-ene-3,20-dione (No. 18)

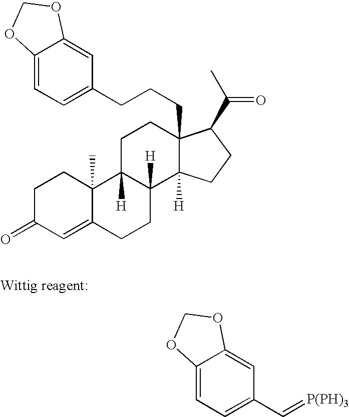

### 18-[2-benzo[1,3]dioxol-5-yl-ethyl]-(9β,10α)-pregna-4,6-diene-3,20-dione (No. 19)

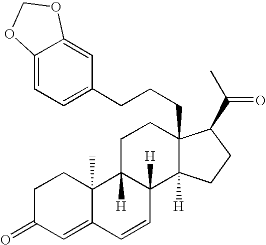

¹H-NMR (500 MHz, CDCl₃): δ 6.65 (m, 3H, arom. H); 6.1 (m, 2H, H-5, H-6); 5.9 (s, 2H, CH₂-Acetal); 5.6 (s, 1H, H-4); 2.95 (m, 1H); 2.05 (s, 3H, 21-CH₃); 1.1 (s; 3H, 19-CH₃); 2.6-0.9 (m, aliph. H).

### 18-[2-(3,4-Difluoro-phenyl)-ethyl]-(9β,10α)-pregna-4-ene-3,20-dione (No. 20)

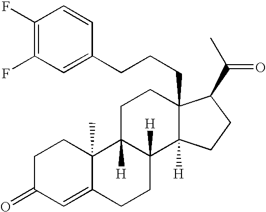 Wittig reagent: Ph₃P═CH-(3,4-difluorophenyl)

### 18-[2-(3,4-Difluoro-phenyl)-ethyl]-(9β,10α)-pregna-4,6-diene-3,20-dione (No. 21)

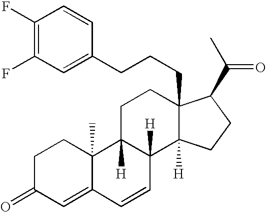

¹H-NMR (500 MHz, CDCl₃): δ 7.0 (ddd, 1H, arom. H); 6.9 (ddd, 1H, arom. H); 6.8 (m, 1H, arom. H); 6.1 (m, 2H, H-5, H-6); 5.6 (s, 1H, H-4); 2.9 (d, 1H); 2.5 (m, 2H, allyl. H); 2.5-0.85 (m, aliph. H); 2.0 (s, 3H, 21-CH₃); 1.1 (s; 3H, 19-CH₃).

### 18-[2-Pyridin-3-yl-ethyl]-(9β,10α)-pregna-4-ene-3,20-dione (No. 22)

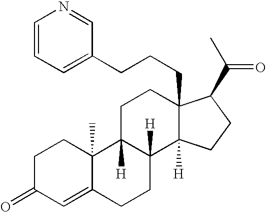 Wittig reagent: Ph₃P═CH-(pyridin-3-yl)

### 18-[2-Pyridin-3-yl-ethyl]-(9β,10α)-pregna-4,6-diene-3,20-dione (No. 23)

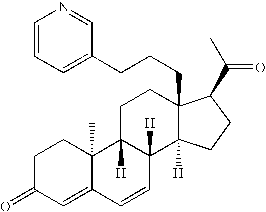

¹H-NMR (500 MHz, CDCl₃): δ 8.7 (s, 1H, arom. H); 8.6 (d, 1H, arom. H); 8.15 (d, 1H, arom. H); 7.8 (t, 1H, arom. H); 6.1 (m, 2H, H-5, H-6); 5.6 (s, 1H, H-4); 2.95 (m, 1H, aliph. H); 2.85 (m, 2H, allyl. H); 2.5-0.9 (m, aliph. H); 2.05 (s, 3H, 21-CH₃); 1.1 (s; 3H, 19-CH₃).

### 18-[2-(3-Methoxy-Phenyl)-ethyl]-(9β,10α)-pregna-4-ene-3,20-dione (No. 24)

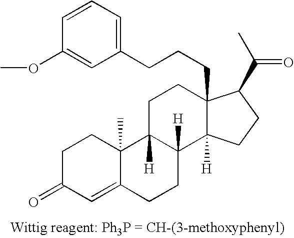

### 18-[2-(3-Methoxy-phenyl)-ethyl]-(9β,10α)-pregna-4,6-diene-3,20-dione (No. 25)

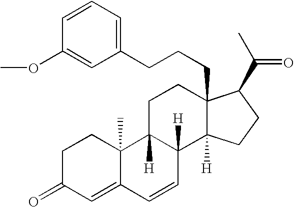

¹H-NMR (500 MHz, CDCl₃): δ 7.15 (m, 1H, arom. H); 6.75 (m, 1H, arom. H); 6.7 (m, 2H, arom. H); 6.1 (m, 2H, H-5, H-6); 5.6 (s, 1H, H-4); 3.75 (s, 3H, OMe); 2.95 (m, 1H); 2.6 (m, 2H, allyl. H); 2.5-0.9 (m, aliph. H); 2.0 (s, 3H, 21-CH₃); 1.1 (s; 3H, 19-CH₃).

### 18-[2-(4-Methoxy-phenyl)-ethyl]-(9β,10α)-pregna-4-ene-3,20-dione (No. 26)

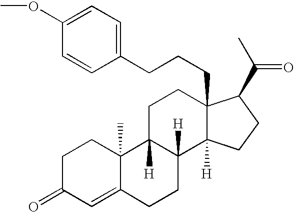 Wittig reagent: Ph₃P═CH-(4-methoxyphenyl)

### 18-[2-(4-Methoxy-phenyl)-ethyl]-(9β,10α)-pregna-4,6-diene-3,20-dione (No. 27)

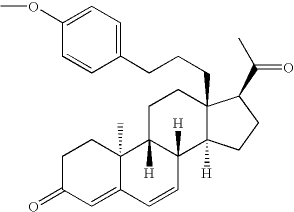

¹H-NMR (500 MHz, CDCl₃): δ 7.05 (d, 2H, arom. H); 6.8 (d, 2H, arom. H); 6.1 (m, 2H, H-5, H-6); 5.6 (s, 1H, H-4); 3.7 (s, 3H, OMe); 2.95 (m, 1H); 2.55 (m, 2H, allyl. H); 2.5-0.9 (m, aliph. H); 2.0 (s, 3H, 21-CH₃); 1.1 (s; 3H, 19-CH₃).

### 18-[2-(3,5-Dimethoxy-phenyl)-ethyl]-(9β,10α)-pregna-4-ene-3,20-dione (No. 28)

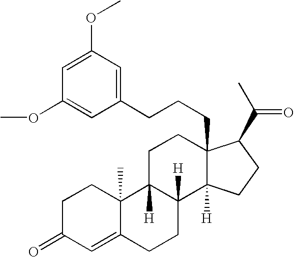 Wittig reagent: Ph₃P═CH-(3,5-dimethoxyphenyl)

### 18-[2-(3,5-Dimethoxy-phenyl)-ethyl]-(9β,10α)-pregna-4,6-diene-3,20-dione (No. 29)

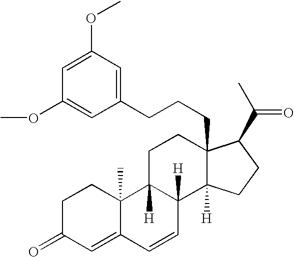

¹H-NMR (500 MHz, CDCl₃): δ 6.35-6.25 (m, 3H, arom. H); 6.1 (m, 2H, H-5, H-6); 5.6 (s, 1H, H-4); 3.73 (s, 6H, 2 OMe); 2.95 (m, 1H); 2.6-0.9 (aliph. H); 2.0 (s, 3H, 21-CH₃); 1.15 (s; 3H, 19-CH₃).

### 18-[2-(3-Trifluoro-methoxy-Phenyl)-ethyl]-(9β,10α)-pregna-4-ene-3,20-dione (No. 30)

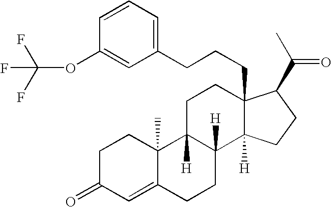 Wittig reagent: Ph₃P═CH-(3-trifluoromethoxyphenyl)

### 18-[2-(3-Trifluoro-methoxy-Phenyl)-ethyl]-(9β,10α)-pregna-4,6-diene-3,20-dione (No. 31)

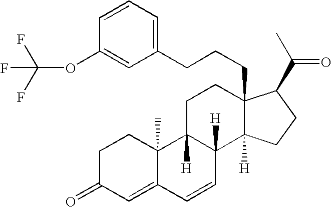

¹H-NMR (500 MHz, CDCl₃): δ 7.25 (m, 1H, arom. H); 7.07 (m, 1H, arom. H); 7.0 (m, 2H, arom. H); 6.1 (dd, 1H, H-6); 6.07 (d, 1H, H-5); 5.6 (s, 1H, H-4); 2.9 (m, 1H); 2.6 (m, 2H, allyl. H); 2.5-0.9 (m, aliph. H); 2.0 (s, 3H, 21-CH₃); 1.15 (s; 3H, 19-CH₃).

### 18-[2-[4-(morpholine-4-carbonyl)-phenyl]-ethyl]-(9β,10α)-pregna-4-ene-3,20-dione (No. 32)

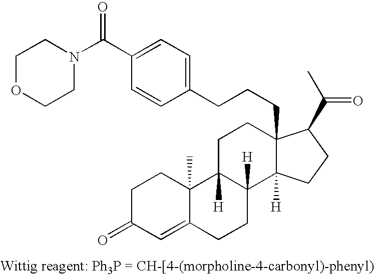

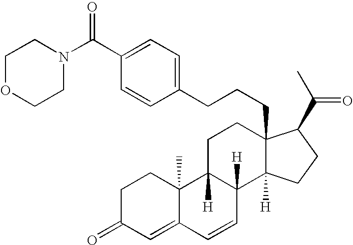

### 18-{2-[4-(morpholine-4-carbonyl)-phenyl]-ethyl}-(9β,10α)-pregna-4,6-diene-3,20-dione (No. 33)

¹H-NMR (500 MHz, CDCl₃): δ 7.3 (d, 1H, arom. H); 7.25 (d, 1H, arom. H); 7.2 (d, 1H, arom. H); 7.1 (d, 1H, arom. H); 6.1 (m, 2H, H-5, H-6); 5.6 (s, 1H, H-4); 3.6 (m, 8H, Morpholino-H); 2.95 (m, 1H); 2.6 (m, 2H, allyl. H); 2.6-0.9 (aliph. H); 2.1 (s, 3H, 21-CH₃); 1.2 (s; 3H, 19-CH₃).

### Biological Testing Materials and Methods

All pharmacological animal data provided herein shall be understood as predictive for the effectiveness of the compounds, compositions, uses and methods of treating thereof, in humans.

I. Progesterone Receptor Binding Assay

The progesterone receptor (PR) binding assays were performed at CEREP (Celle I'Evescault, France).

Procedure:

The binding to the bovine progesterone receptor (Assay Cat No: Progesterone receptor-814) was measured using 3H-R5020 as ligand and uterus tissue as the source of progesterone receptor. The assay was performed as described by Hurd & Moudgil [1988].

The binding to the human progesterone receptor (Assay Cat No: Progesterone receptor-814h) may be measured using 3H-R5020 as ligand and MCF7 cells as the source of progesterone receptor. The assay is performed as described by Eckert & Katzenellenbogen [1982].

The assay does not discriminate between the two progesterone receptor isoforms PRa and PRβ.

Results

The results of the receptor binding assay are presented as the individual pKi values for the bovine PR which were determined by measuring the binding activity for a concentration range of each compound. The data for selected compounds are summarized in the following Table:

| Compound | | Progesterone Receptor |
|---|---|---|
| No. | Compound Structure | binding (pKi) |
| 1 | 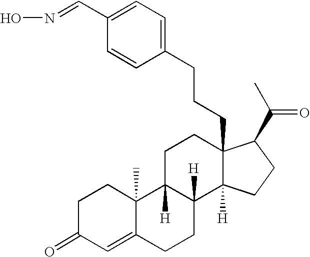 | 6.0 (bovine) |
| 2 | 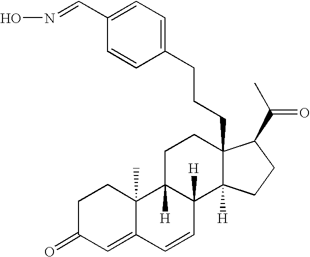 | 6.7 (bovine) |
| 4 | 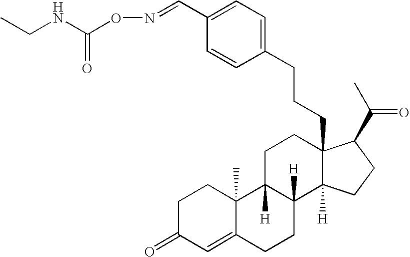 | 6.3 (bovine) |
| 5 | 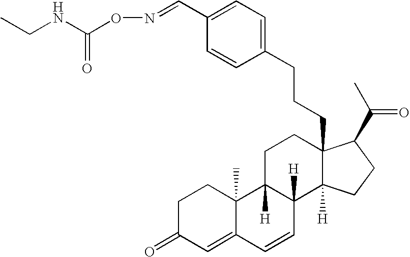 | 6.7 (bovine) |
| 6 | 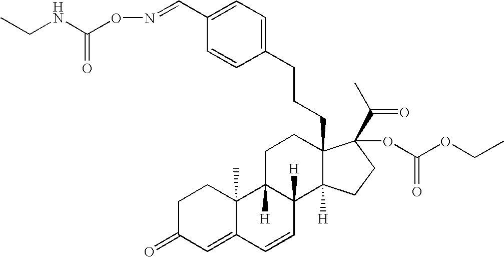 | 6.3 (bovine) |
| 13 | 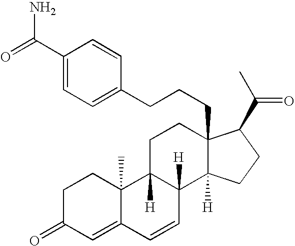 | 6.7 (bovine) |

II. Progesterone-dependent Alkaline Phosphatase Expression Assay

The progesterone-dependent modulation of alkaline phosphatase expression was examined using T47D human breast carcinoma cells [Keydar et al., 1979]. The assay was performed as previously described by Di Lorenzo et al. (1991) with the modification of using Dydrogesterone as comparative progestin to determine the antagonistic and agonistic activity.

Procedure

The cell line was purchased from CLS Cell Lines Service (Hildastrasse 21, D-69214 Eppelheim, Germany).

In brief, the cells were plated in 96-well plates at 40,000 cells/well using the following growth medium: RPMI 1640 with: 10% FBS, 1 mM Sodium Pyruvat MEM, 10 mM Hepes, 0.01 mg/ml Bovine insulin, and 25 μg/ml Gentamycin. After 24 h of cultivation, the growth medium was replaced with medium containing 2% fetal bovine serum and the test compounds were added to each well to achieve the appropriate compound concentration: For determination of agonistic activity only the test compounds were added; for measurement of antagonistic activity the test compounds and additionally Dydrogesterone as standard progesterone agonist was added to a final concentration of 1 nM. After 48 h of cultivation, the medium was removed and the cells were washed with 200 μl of Dulbecco's phosphate-buffered saline without calcium and magnesium (PBS(−)).

Then the cells were fixed with 3.7% formaldehyde in phosphate-buffered saline for 15 min at 22° C. After washing the cells with PBS, 100 μl of a para-nitro-phenole (pNPP) solution (pNPP Liquid Substrate System; Sigma) was added to each well and incubated for 2 h at room temperature protected from light. The reaction was stopped with 100 μl 1N NaOH and the absorbance was measured with a spectrophotometer (Victor, Perkin Elmer) at 405 nm.

The results are expressed as alkaline phosphatase induction (as 100% with 1 nM Dydrogesterone) or inhibition (against alkaline phosphatase induction by 1 nM Dydrogesterone) at a certain concentration of test compound.

Calculations:
% stimulation=(effect compound−basal)/(effect dydro 1 nM−basal)*100
% inhibition of 1 nM Dydro=100*{1−[(effect compound·basal)/(effect dydro 1 nM·basal)]}

For each compound the % inhibition (PI) and % stimulation (PS), respectively, at a compound concentration of 100 nM was determined. For selected compounds, the corresponding values were measured for several different concentrations, and subsequently were plotted against the concentration of the test compound, and used to calculate the IC50 value (for the antagonistic potency; the IC50 value is the concentration (nM), required to reduce the maximal response by 50%) and EC50 values (for the agonistic potency; the EC50 value is the effective concentration (nM) that produced 50% of the maximum response), respectively.

Results

The results of the AP assay are presented in the following table.

| | | AP Assay Results | | | |
|---|---|---|---|---|---|
| | | PI | PS | | |
| Compound No. | Compound Structure | [100 nM] | [100 nM] | pIC50 | pEC50 |
| 1 | 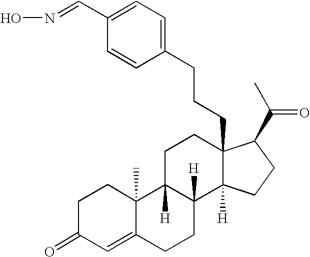 | 60 | 1 | 7.5 | |
| 2 | 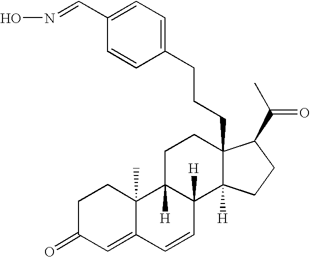 | 77 | 10 | 7.7 | |
| 4 | 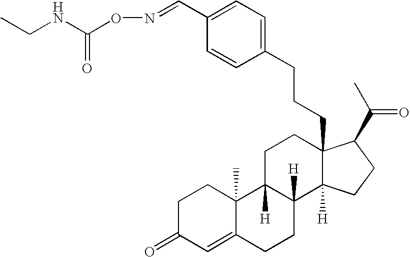 | 51 | 4 | 7.3 | |
| 5 | 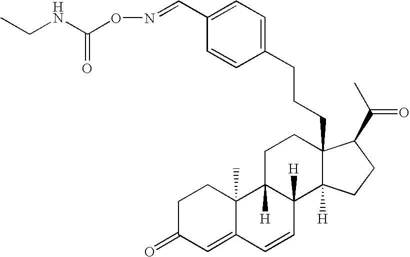 | 52 | 35 | 8.3 | |
| 6 | 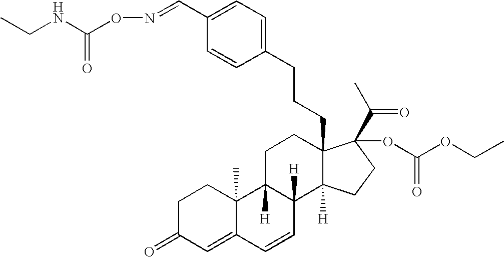 | 32 | 60 | | 7.3 |
| 13 | 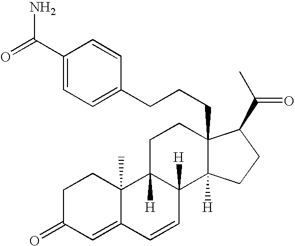 | 79 | 7 | 7.3 | |
| 17 | 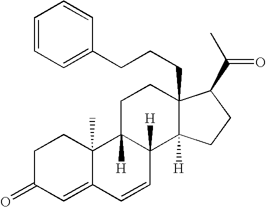 | 79 | 8 | | 7.6 |
| 25 | 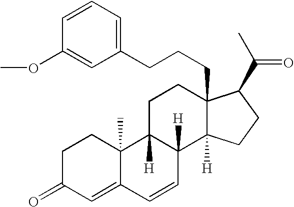 | 73 | 12 | | 7.8 |
| 29 | 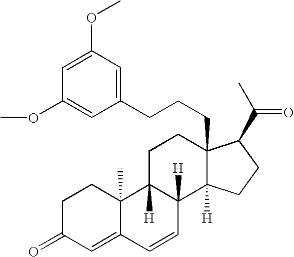 | 69 | 15 | | 7.8 |

III. Clauberg-McPhail Assay

The in vivo activity of selected PR modulator compounds of the present invention was evaluated utilizing the McPhail assay. The Clauberg or McPhail assay is a classic assay utilizing rabbits to measure progestational activity and allows the assessment of the progestagenic and antiprogestagenic effects of the compounds [McPhail, 1934]. The reason rabbit is used is because the results observed in rabbit have proved to be a good indicator and predictor of activity in the human. In this assay, immature rabbits are treated initially with estradiol, which induces growth in the uterus. This is followed by treatment with a progestin, which causes a large change in the glandular content of the uterus. It is this change in the glandular component, which is a measure of the progestational activity of a progestin. The measurement of these glandular changes is carried out histologically using stained sections of the uterus.

Procedure

The test is performed in 6-week-old juvenile female rabbits (New Zealand white). From days 1 to 6, all rabbits are primed with 5.0 μg/kg/day 17β-estradiol (s.c., 0.5 ml/kg/day) in order to induce proliferation of the endometrium. From days 7 to 11, the test compound is applied (0.5 ml/kg/day) at doses in the range of 0.001 to 10 mg/kg/day. A group which receives only vehicle after estradiol priming serves as a negative control. A second group which receives only progesterone in order to induce endometrial differentiation after estradiol priming is used as a positive control. The antagonistc activity is measured by the combined administration of progesterone and the test compound in the appropriate dosages.

Evaluation

Autopsy is performed on day 12. As a parameter for progestagenic activity, the McPhail index (i.e., the degree of differentiation) is determined by means of light microscopy (scores: 1 to 4; 1=no glandular differentiation, 4=maximal differentiation).

By definition, progesterone produces a maximum McPhail score of 4; treatment with a PR antagonist in the absence of progesterone leads to a McPhail score which is distinctly lower in score than 4 at the plateau of the dose response curve at the clinically relevant doses (i.e. 0.01 mg-10 mg/rabbit). Preferably, a SPRM leads to a McPhail score which is higher than that under any dose of RU 486 (Mifepristone), i.e. above 0.5-1.0, preferentially above 2.0-3.0. The capacity of SPRMs to antagonize progesterone function can also be tested in the McPhail test using a progesterone dose which induces a McPhail score ranging between 3 and 4. A SPRM inhibits the effect of progesterone to a significant degree, but the maximum inhibition is below that which is inducible with RU 486 or other pure antiprogestins, such as onapristone.

Results

The preferred compounds of the invention lead to a McPhail score which is above 0.5-1.0, preferentially above 2.0-3.0. In the antagonistic mode, the preferred compounds of the invention show a significant inhibition of the effect of the administered progesterone; however, they show a maximum inhibition clearly below that which is inducible with pure antiprogestins.

IV. Guinea Pig Model

An assay how to assess the progesterone antagonists (PAs) and progesterone receptor modulators (PRMs) with respect to PR agonistic and antagonistic activities in vivo is described by Elger et al [2000] and within WO 04/014935 using cycling guinea pigs. In this assay, pure PR antagonists inhibit luteolysis at the end of the ovarian cycle, whereas PR agonist and SPRMs support luteolysis, i.e. this is a very sensitive in vivo method to reveal residual agonistic activity of SPRMs. Inhibition of luteolysis is reflected by elevated serum progesterone levels at day 10-17 and inhibition of uterine prostaglandin F2_{α}, as well as by certain histological characteristics in uterus and ovary, such as increased expression of progesterone receptors and decreased glandular differentiation in the uterus, as well as persistence of large intact corpora lutea up to day 18.

Procedure

Adult female guinea pigs (strain Dunkin Hartley, Crl:HA; body weight 500-700 g) are purchased from Charles River (Sulzfeld, Germany). Blood samples are drawn from the Vena saphena three times a week to monitor ovarian cycles by determination of progesterone levels. Animals showing at least two regular ovarian cycles are treated once daily with 10mg/kg s.c. of test compounds dissolved in benzyl benzoate/castor oil (1+4 vol), on days 10-17 of the cycle. During this treatment period blood samples for progesterone determination are collected once daily. On day 18 the animals are killed by CO₂-asphyxiation. Ovaries and uteri are collected and processed for histological analysis.

Evaluation

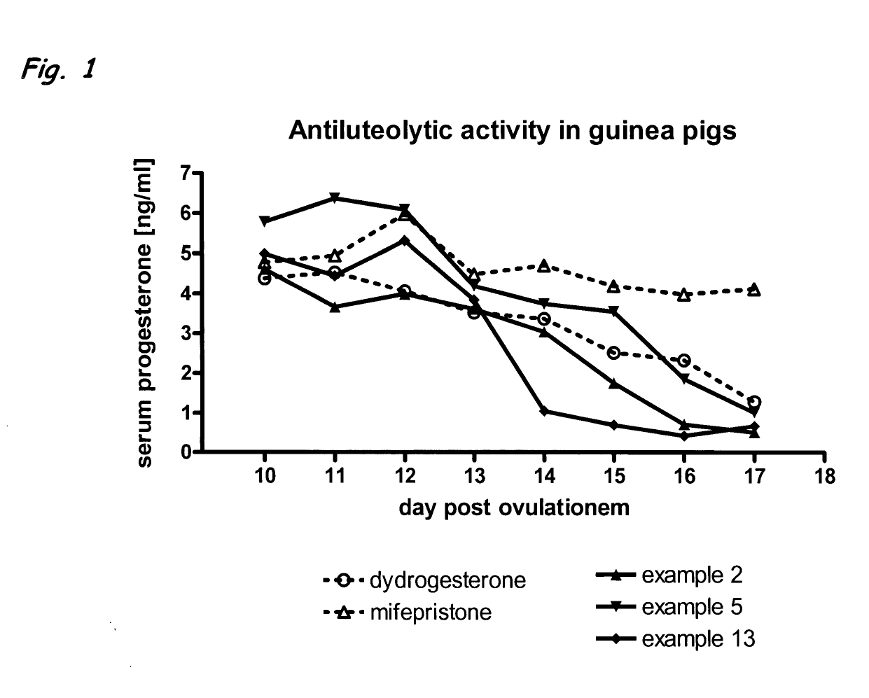

Antiluteolytic activity is evaluated by assessment of serum progesterone profiles throughout the treatment period from day 10 to day 17 (FIG. 1). Progesterone levels do not decline, i.e. luteolysis is inhibited, when antiprogestins like mifepristone (RU486) are administered. With progestins (e.g. dydrogesterone) and SPRMs, progesterone levels decrease meaning that no inhibition of luteolysis is observed.

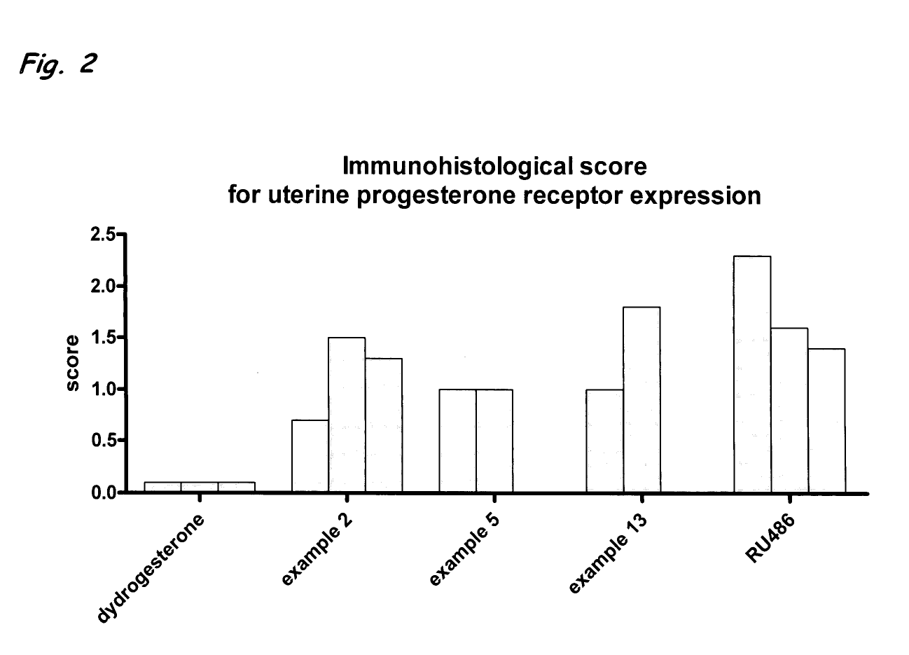

Antiprogestational effects on the uterus are assessed by determination of the degree of PR expression. PR is stained by immunohistochemistry in 5-μm cross-sections of the uteri using the DAKO Envision method according to the manufacturer's instructions and mouse-anti-human progesterone receptor antibody (1:20 dilution, DAKO Diagnostika, Hamburg, Germany). A minimum histological score of 0 is assigned to sections with no PR expression, while a maximum score of 3 is assigned to strong PR expression as judged by staining intensity and number of PR-positive cells (FIG. 2; 1 bar represents one animal). PR-agonists reduce uterine PR expression, whereas PR-antagonists block the PR and increase PR expression.

Results

Like known agonists and SPRMs the compounds of the invention support luteolysis (FIG. 1), but unlike pure agonists they do not decrease uterine PR expression (FIG. 2).

V. Summary

The compounds and pharmaceutical compositions of the present invention may be extremely potent modulators of the PR, while however their absolute agonistic activity remains below that of natural progesterone in the plateau of the dose response curve and their absolute antagonistc activity remains below that of known antiprogestins such as onapristone or mifepristone (RU 486).

For example, the compounds and compositions of the present invention may display 50% maximal activation of the progesterone receptor at a concentration of less than 10 μM. Some compounds and compositions of the present invention may display 50% maximal activation of PR at a concentration of less than 1 μM, and some may display such activity at a concentration of less than 100 nM or even 10 nM.

In a further preferred embodiment of the present invention, the compounds provide for 50% maximum inhibition measured in the antagonistic mode of the AP assay at a concentration of less than 1 μM, preferably less than 100 nM and even more preferred less than 10 nM, and additionally for 50% maximum activation measured using the agonistic AP assay as described here within at a concentration of less than 10 μM, preferably less than 1 μM and even more preferred less than 100 nM.

### Illustrative Pharmaceutical Compositions

The following examples provide illustrative pharmaceutical composition formulations:

I. Hard Gelatin Capsules

Hard gelatin capsules are prepared using the following ingredients:

| Ingredient | Quantity (mg/capsule) |
|---|---|
| COMPOUND No. 5 | 1 |
| Starch, dried | 105 |
| Magnesium stearate | 14 |
| Total | 120 |

The above ingredients are mixed and filled into hard gelatin capsules in 120 mg quantities.

II. Tablets

A tablet is prepared using the following ingredients:

| Ingredient | Quantity (mg/tablet) |
|---|---|
| COMPOUND No. 5 | 1 |
| Cellulose, microcrystalline | 209 |
| Silicon dioxide, fumed | 10 |
| Stearic acid | 10 |
| Total | 230 |

The components are blended and compressed to form tablets each weighing 230 mg.

III. Suppositories

Suppositories, each containing 1 mg of active ingredient, may be made as follows:

| Ingredient | Quantity (mg/suppository) |
|---|---|
| COMPOUND No. 5 | 1 |
| Saturated fatty acid glycerides | 2,000 |
| Total | 2,001 |

The active ingredient is passed through a appropriately sized mesh sieve and suspended in the saturated fatty acid glycerides previously melted using the minimum heat necessary. The mixture is then poured into a suppository mold of normal 2 g capacity and allowed to cool.

IV. Intravenous Formulation

An intravenous formulation may be prepared as follows:

| Ingredient | Quantity | |
|---|---|---|
| COMPOUND No. 5 | 5 | mg |
| Isotonic saline | 1000 | ml |
| Glycerol | 100 | ml |

The compound is dissolved in the glycerol and then the solution is slowly diluted with isotonic saline.

General Provisions

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

Cited Literature

Citation of any reference throughout this application is not to be construed as an admission that such reference is prior art to the present application.
BE 577,615
BE 652,597
Di Lorenzy D.; Albertine A.; Zava D. (1991): Progestin regulation of alkaline phosphatase in the human breast cancer cell line T47D. Cancer Research 51: 4470-4475
Eckert RL & Katzenellenbogen BS (1982) “Effects of estrogens and antiestrogens on estrogen receptor dynamics and the induction of progesterone receptor in MCF-7 human breast cancer cells.” Cancer Res. 42(1):139-44
Elger W, Bartley J, Schneider B, Kaufmann G, Schubert G, Chwalisz K. (2000) “Endocrine pharmacological characterization of progesterone antagonists and progesterone receptor modulators with respect to PR-agonistic and antagonistic activity”. Steroids, 65(10-11):713-23
EP 0152138B1 (U.S. Pat. No. 4,601,855)
EP 0558119B1 (U.S. Pat. No. 5,304,291)
EP 0648778 (U.S. Pat. No. 5,693,628)
EP 1229906 (WO 01/15679)
EP 0909764
EP 0152138B1 (U.S. Pat. No. 4,601,855)
EP 0558119B1 (U.S. Pat. No. 5,304,291)
Halkes S J, Hartog J, Morsink L, de Wachter A M. (1972) “Investigations on sterols. 38. Synthesis of 1,2-methylene-17α-acetoxy-9β,10α-pregnanes, a class of potent progestational agents” J Med Chem, 15(12):1288-92
Halkes S J & Van Moorselaar R (1969) “Investigations on Sterols XXXIV: Synthesis of 18-methyl-9β,10α-androstanes” Recueil des Travaux Chimiques des Pays-Bas, 1969, 88(7):752-765
Hartog J, Wittelaar J I, Morsink L, de Wachter A M (1972) “Investigations on sterols. 39. Synthesis and progestational activities of some 16-methylene-17α-acetoxy-9β,10α-pregna-4,6-diene-3,20-dione derivatives” J Med Chem. 1972 Dec; 15(12):1292-7
Hurd C & Moudgil V K (1988) “Characterization of R5020 and RU486 binding to progesterone receptor from calf uterus.” Biochemistry, 27(10):3618-23
Keydar, l. et al. (1979): Establishment and characterization of a cell line of human breast carcinoma origin. Eur. J. Cancer 15: 659-670.
McPhail M K (1934) “The assay of progestin” J Physiol 83:145-156
Mencaglia, L., Perino, A., Hamon, J. (1987) Hysteroscopy in Perimenopausal and Postmenopausal Women with Abnormal Uterine Bleeding. J. Reprod. Med. 32:577
Schubert G, Elger W, Kaufmann G, Schneider B, Reddersen G, Chwalisz K. (2005) “Discovery, chemistry, and reproductive pharmacology of asoprisnil and related 11beta-benzaldoxime substituted selective progesterone receptor modulators (SPRMs).” Semin Reprod Med. 23(1):58-73. Review
Sobek L, Di Lorenzo D, Oettel M, Kaufmann G. (1994) “Normal and stable transfected cancer cell lines: tools for a screening of progestogenic, antiprogestogenic and antiglucocorticoid substances.” Methods Find Exp Clin Pharmacol. 16(7):545-51. Review
Spitz IM. “Progesterone antagonists and progesterone receptor modulators: an overview” Steroids. 2003 68(10-13):981-93
Spitz IM. “Progesterone antagonists and progesterone receptor modulators” Expert Opin Investig Drugs. 2003 12(10):1693-707. Review
Spitz IM. “Progesterone receptor antagonists and selective progesterone receptor modulators (SPRMs)” Semin Reprod Med. 2005 23(1):3-7
T. W. Greene & P. G. M. Wuts “Protective groups in Organic Synthesis” John Wiley & Sons
Tamaya T, Fujimoto J, Okada H. “Comparison of cellular levels of steroid receptors in uterine leiomyoma and myometrium.” Acta Obstet Gynecol Scand. 1985;64(4):307-9
U.S. Pat. No. 3,304,314
U.S. Pat. No. 3,555,053
U.S. Pat. No. 3,937,700
Van Moorselaar R. & Halkes SJ (1969) “Investigations on Sterols XXXIII: Synthesis of 18-alkyl-9β,10α-pregnane derivatives” Recueil des Travaux Chimiques des Pays-Bas, 88(7):737-51
Westerhof P. & Hartog J. “Investigations on Sterols XXIX: Synthesis and properties of some 6,7-dehydro-9β,10α-steroids” Recueil des Travaux Chimiques des Pays-Bas 1965, 84(7):91 8-31
Westerhof P., Hartog J. & Halkes S J (1965) “Investigations on Sterols XXVI: Synthesis and properties of 6-substituted 9β,10α-steroids” Recueil des Travaux Chimiques des Pays-Bas, 84:863-884.
WO 00/34306
WO 001/15679
WO 01/18025
WO 01/44267
WO 02/054064
WO 03/093292
WO 04/014935
WO 99/45022
WO 99/45023
WO 99/62928
WO 99/62929
Zahradnik (1992) “Menstruation”. In Kaser O et al. (editors) Gynaekologie und Geburtshilfe [Gynecology and Obstetrics], Vol. ½, Georg Thieme Verlag Stuttgart, New York: 7.31-7.51
Zhang P, Fensome A, Wrobel J, Winneker R, Zhang Z (2003) “Non-steroidal progesterone receptor modulators” Expert Opin Ther Patents 13(12):1839-1847

What is claimed is:

1. A compound corresponding to formula (I): 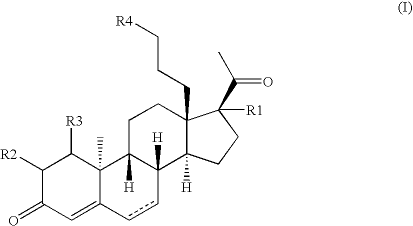
wherein
R1 is selected from the group consisting of hydrogen, —OH, —O—(C₁-C₄)alkyl, —O—CO—(C₁-C₄)alkyl, and —O—CO—O—(C₁-C₄)alkyl;
R2 and R3 are both hydrogen or together form a methylene group;
R4 is selected from the group consisting of —O—R⁶, heteroaryl and aryl;
wherein any heteroaryl or aryl group is optionally substituted with one or two substituents independently selected from the group consisting of
—CHO; —CO—O—R⁹, —CO—NR¹²R¹³, —CH₂—O—R⁹; —CH₂—O—CO—R¹¹, —CH₂—O—CO—NHR¹², —CH═N—O—R¹⁴, —CH═N—O—CO—NHR¹², —CH═N—O—CO—R¹¹, —CH═N—O—CO—O—R¹⁴; —CN; —CH₂—NH—CO—NHR¹², —CH₂—NH—CO—R¹¹, —CH₂—NH—CO—O—R¹⁴, -halogen, —O—R⁹, —O—CO—R¹¹; —O—CO—NHR¹², —NR¹²R¹³; —NR¹⁰—O—CO—R¹¹, —NR¹⁰—CO—NHR¹², —NR¹⁰—CO—O—R¹⁴, —(C₁-C₄)alkyl, and halogenated —(C₁-C₄)alkyl,
or any aryl group is optionally substituted by two groups attached to adjacent carbon atoms and combined into a saturated or partly unsaturated cyclic 5, 6, 7, or 8 membered ring system, optionally containing 1, 2 or 3 heteroatoms selected from the group consisting of N, O and S, the number of N atoms being 0, 1, 2 or 3 and the number of O and S atoms each being 0, 1 or 2;
R⁶, R⁹, R¹⁰, R¹¹, R¹², R¹³ and R¹⁴ are independently selected from the group consisting of hydrogen, —(C₁-C₄)alkyl, and halogenated —(C₁-C₄)alkyl; or
R¹² and R¹³ together with the nitrogen atom to which R¹² and R¹³ are attached, form a heterocyclic 4-, 5-, 6-, 7- or 8-membered ring system, which is saturated, partly unsaturated, or aromatic, and which optionally contains 1, 2 or 3 additional heteroatoms selected from the group consisting of N, O and S, the number of additional N atoms being 0, 1, 2 or 3 and the number of O and S atoms each being 0, 1 or 2; and which ring is optionally part of a multiple condensed ring-system;
or a salt, tautomer, stereoisomer, or pro-drug thereof.

2. A compound according to claim 1, wherein
R4 is selected from the group consisting of —O—R⁶, heteroaryl and aryl,
wherein any aryl group is optionally substituted with one or two substituents independently selected from the group consisting of
—CHO; —CO—O—R⁹, —CO—NR¹²R¹³, —CH₂—O—R⁹; —CH═N—O—R¹⁴, —CH═N—O—CO—NHR¹², —CH═N—O—CO—R¹¹, —CH═N—O—CO—O—R¹⁴, —CH₂—NH—CO—NHR¹², —CH₂—NH—CO—R¹¹, —CH₂—NH—CO—O—R¹⁴, -halogen and —O—R⁹,
or wherein any aryl group is optionally substituted by two groups attached to adjacent carbon atoms and combined into a saturated or partly unsaturated cyclic 5, 6 or 7-membered ring system, optionally containing 1 or 2 heteroatoms selected from the group consisting of N and O, the number of N atoms being 0, 1 or 2, and the number of O atoms being 0, 1 or 2;
R⁶, R⁹, R¹¹, R¹², R¹³ and R¹⁴ are independently selected from the group consisting of hydrogen, —(C₁-C₄)alkyl, and halogenated —(C₁-C₄)alkyl; or
R¹² and R¹³ together with the nitrogen atom to which they are attached, form a heterocyclic 5-, 6- or 7-membered ring system, which is saturated or partly unsaturated, and which optionally contains 1 or 2 additional heteroatoms selected from the group consisting of N and O, the number of additional N atoms being 0, 1 or 2, and the number of 0 atoms being 0 or 1.

3. A compound according to claim 2, wherein
R1 is selected from the group consisting of hydrogen and —O—CO—O—(C₁-C₄)alkyl;
R2 and R3 are both hydrogen;
R4 is selected from the group consisting of —OH, -phenyl, furyl and pyridyl,
wherein any phenyl group is optionally substituted with one or two substituents in meta- or para-position or both meta- and para-position independently selected from the group consisting of
—CHO; —CO—O—R⁹, —CO—NR¹²R¹³, —CH₂—O—R⁹; —CH═N—O—R¹⁴, —CH═N—O—CO—NHR¹², -halogen and —O—R⁹;
or wherein any phenyl group is optionally substituted by two groups attached to adjacent carbon atoms and combined into a saturated cyclic 5-, 6- or 7-membered ring system, optionally containing 1 or 2 O atoms; and
R⁹, R¹², R¹³ and R¹⁴ are independently selected from the group consisting of hydrogen, —(C₁-C₄)alkyl, and halogenated —(C₁-C₄)alkyl; or
R¹² and R¹³ together with the nitrogen atom to which they are attached, form a saturated heterocyclic 5-, 6- or 7-membered ring system, which optionally contains 1 additional heteroatom selected from the group consisting of N and O.

4. A compound according to claim 1, corresponding to formula (II)

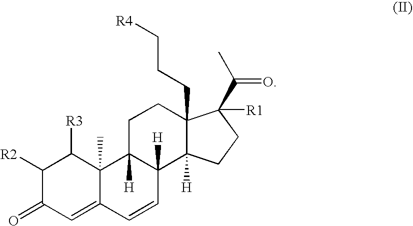

5. A compound according to claim 1, corresponding to formula (III) 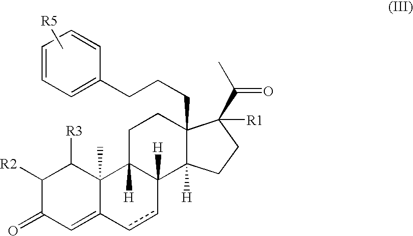
wherein
R1 is selected from the group consisting of hydrogen, —OH, —O—(C₁-C₄)alkyl, —O—CO—(C₁-C₄)alkyl, and —O—CO—O—(C₁-C₄)alkyl;
R2 and R3 are both hydrogen or together form a methylene group;
R5 is selected from the group consisting of —CHO; —CO—O—R⁹, —CO—NR¹²R¹³, —CH₂—O—R⁹; —CH₂—O—CO—R¹¹; —CH₂—O—CO—NHR¹²; —CH═N—O—R¹⁴, —CH═N—O—CO—NHR¹², —CH═N—O—CO—R¹¹, —CH═N—O—CO—O—R¹⁴, —CN; —CH₂—NH—CO—NHR¹², —CH₂—NH—CO—R¹¹, —CH₂—NH—CO—O—R¹⁴, -halogen, —O—R⁹, —O—CO—R¹¹, —O—CO—NHR¹², —NR¹²R^{13, —NR}¹⁰—CO—R¹¹, —NR¹⁰—CO—NHR¹², —NR¹⁰—O—CO—O—R¹⁴, —(C₁-C₄)alkyl, and halogenated —(C₁-C₄)alkyl;
R⁹, R¹⁰, R¹¹, R¹², R¹³ and R¹⁴ are independently selected from the group consisting of hydrogen, —(C₁-C₄)alkyl, and halogenated —(C₁-C₄)alkyl; or
R¹² and R¹³ together with the nitrogen atom to which R¹² and R¹³ are attached form a heterocyclic 4-, 5-, 6-, 7- or 8-membered ring system, which is saturated, partly unsaturated, or aromatic; and which optionally contains 1, 2 or 3 additional heteroatoms selected from the group consisting of N, O and S, the number of additional N atoms being 0, 1, 2 or 3 and the number of O and S atoms each being 0, 1 or 2; and which ring is optionally part of a multiple condensed ring-system.

6. A compound according to claim 5, corresponding to formula (III) wherein
R1 is selected from the group consisting of hydrogen and —O—CO—O—(C₁-C₄)alkyl;
R2 and R3 are both hydrogen;
R5 is selected from the group consisting of —CHO; —CO—O—R⁹, —CO—NR¹²R¹³, —CH₂—O—R⁹; —CH═N—O—R¹⁴, —CH═N—O—CO—NHR¹², -halogen, and —O—R⁹, and
R⁹, R¹², R¹³ and R¹⁴ are independently selected from the group consisting of hydrogen, —(C₁-C₄)alkyl and halogenated —(C₁-C₄)alkyl, or
R¹² and R¹³ together with the nitrogen atom to which R¹² and R¹³ are attached form a heterocyclic 5-, 6- or 7-membered ring system, which is saturated or partly unsaturated; and which optionally contains 1 or 2 additional heteroatoms selected from the group consisting of N and O, the number of additional N atoms being 0, 1 or 2, and the number of O atoms being 0 or 1.

7. A compound according to claim 5, corresponding to formula (V)

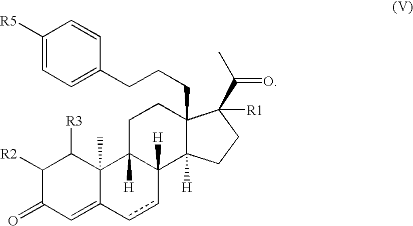

8. A compound according to claim 1, selected from the group consisting of:
18-[2-(4-oximino-formylphenyl)-ethyl]-((9β,10α)-pregna-4-ene-3,20-dione (No. 1),
18-[2-(4-oximino-formylphenyl)-ethyl]-((9β,10α)-pregna-4,6-diene-3,20-dione (No. 2),
18-[2-(4-oximino-formylphenyl)-ethyl]-3,20-dioxo-((9β,10α)-pregna-4,6-diene-17-yl-carbonic acid ethyl ester (No. 3),
18-[2-(4-N-ethylcarbamoyl-oximino-formylphenyl)-ethyl]-((9β,10α)-pregna-4-ene-3,20-dione (No. 4),
18-[2-(4-N-ethylcarbamoyl-oximino-formylphenyl)-ethyl]-((9β,10α)-pregna-4,6-diene-3,20-dione (No. 5),
18-[2-(4-N-ethylcarbamoyl-oximino-formylphenyl)-ethyl]-3,20-dioxo-((9β,10α)-pregna-4,6-diene-17-yl-carbonic acid ethyl ester (No. 6),
18-[2-(4-hydroxymethyl-phenyl)-ethyl]-(9β,10α)-pregna-4-ene-3,20-dione (No. 7),
18-(2-[4-hydroxymethyl-phenyl]-ethyl)-3,20-dioxo-(9β,10α)-pregna-4-ene-17-yl-carbonic acid ethyl ester (No. 8),
18-[2-(4-formyl-phenyl)-ethyl]-(9β,10α)-pregna-4-ene-3,20-dione (No. 9),
18-[2-(4-formyl-phenyl)-ethyl]-(9β,10α)-pregna-4,6-diene-3,20-dione (No. 10),
18-[2-(4-formyl-phenyl)-ethyl]-3,20-dioxo-(9β,10α)-pregna-4-ene-17-yl-carbonic acid ethyl ester (No. 11),
18-[2-(4-formyl-phenyl)-ethyl]-3,20-dioxo-(9β,10α)-pregna-4,6-diene-17-yl-carbonic acid ethyl ester (No. 12),
18-[2-(4-formamido-phenyl)-ethyl]-(9β,10α)-pregna-4,6-diene-3,20-dione (No. 13),
18-[2-(4-formic acid-phenyl)-ethyl]-(9β,10α)-pregna-4-ene-3,20-dione (No. 14),
18-[2-(4-formic acid-phenyl)-ethyl]-(9β,10α)-pregna-4,6-diene-3,20-dione (No. 15),
18-[2-phenyl)-ethyl]-(9β,10α)-pregna-4-ene-3,20-dione (No. 16),
18-[2-phenyl)-ethyl]-(9β,10α)-pregna-4,6-diene-3,20-dione (No. 17),
18-[2-benzo[1,3]dioxol-5-yl-ethyl]-(9β,10α)-pregna-4-ene-3,20-dione (No. 18),
18-[2-benzo[1,3]dioxol-5-yl-ethyl]-(9β,10α)-pregna-4,6-diene-3,20-dione (No. 19),
18-[2-(3,4-difluoro-phenyl)-ethyl]-(9β,10α)-pregna-4-ene-3,20-dione (No. 20),
18-[2-(3,4-difluoro-phenyl)-ethyl]-(9β,10α)-pregna-4,6-diene-3,20-dione (No. 21),
18-[2-pyridin-3-yl-ethyl]-(9β,10α)-pregna-4-ene-3,20-dione (No. 22),
18-[2-pyridin-3-yl-ethyl]-(9β,10α)-pregna-4,6-diene-3,20-dione (No. 23),
18-[2-(3-methoxy-phenyl)-ethyl]-(9β,10α)-pregna-4-ene-3,20-dione (No. 24),
18-[2-(3-methoxy-phenyl)-ethyl]-(9β,10α)-pregna-4,6-diene-3,20-dione (No. 25),
18-[2-(4-methoxy-phenyl)-ethyl]-(9β,10α)-pregna-4-ene-3,20-dione (No. 26),
18-[2-(4-methoxy-phenyl)-ethyl]-(9β,10α)-pregna-4,6-diene-3,20-dione (No. 27),
18-[2-(3,5-dimethoxy-phenyl)-ethyl]-(9β,10α)-pregna-4-ene-3,20-dione (No. 28),
18-[2-(3,5-dimethoxy-phenyl)-ethyl]-(9β,10α)-pregna-4,6-diene-3,20-dione (No. 29),
18-[2-(3-trifluoro-methoxy-phenyl)-ethyl]-(9β,10α)-pregna-4-ene-3,20-dione (No. 30),
18-[2-(3-trifluoro-methoxy-phenyl)-ethyl]-(9β,10α)-pregna-4,6-diene-3,20-dione (No. 31),
18-{2-[4-(morpholine-4-carbonyl)-phenyl]-ethyl}-(9β,10α)-pregna-4-ene-3,20-dione (No. 32), and
18-{2-[4-(morpholine-4-carbonyl)-phenyl]-ethyl}-(9β,10α)-pregna-4,6-diene-3,20-dione (No. 33)

9. A pharmaceutical composition comprising at least one compound according to claim 1, and at least one pharmaceutically acceptable carrier or auxiliary substance.

10. A pharmaceutical composition according to claim 9, further comprising at least one natural or synthetic estrogen or an estrogen pro-drug.

11. A pharmaceutical composition according to claim 10, wherein the estrogen is a natural estrogen.

12. A pharmaceutical composition according to claim 9, wherein the pharmaceutical composition is in the form of an intrauterine device, a transdermal patch or a gel.

13. A method of treating or inhibiting a condition mediated by a progesterone receptor in a patient, said method comprising administering to said patient a pharmaceutically effective amount of a compound according to claim 1.

14. A method according to claim 13, wherein said condition mediated by a progesterone receptor is selected from the group consisting of endometriosis, uterine fibroids, uterine leiomyoma, endometrial hyperplasia, dysmenorrhea, dysfunctional uterine bleeding, menorrhagia, metrorrhagia, hypermenorrhea, hot flashes, mood disorders, meningiomas, hormone-dependent cancer, female osteoporosis, Cushing's syndrome, major depression, neurodegenerative diseases, Alzheimer's disease, and demyelinating diseases.

15. A method according to claim 14, wherein said condition is a hormone-dependent cancer selected from the group consisting of female sex steroid dependent cancer, ovarian cancer, breast cancer, endometrial cancer and prostate cancer.

16. A method according to claim 13, wherein said condition is alleviated with female hormone replacement therapy.

17. A method of modulating fertility in an individual comprising administering to said individual an effective fertility modulating amount of a compound according to claim 1.

18. A method of providing contraception to an individual comprising administering to said individual an effective conception inhibiting amount of a compound according to claim 1.

19. A method of modulating a progesterone receptor in an individual comprising administering to said individual an effective progesterone receptor modulating amount of a compound according to claim 1.

20. A method according to claim 25, wherein said modulation is activation.

21. A method of determining the presence of a progesterone receptor in a cell or cell extract, said method comprising:
(a) labeling a compound according to claim 1;
(b) contacting the cell or cell extract with the labeled compound; and
(c) testing the contacted cell or cell extract to determine the presence of progesterone receptor.
